(19) United States
(12) Patent Application Publication
(10) Pub. No.: US 2018/0049984 A1
(43) Pub. Date: Feb. 22, 2018

(54) Carriers for Plasmid and RNP Delivery in the Treatment of Cancer and Other Disease States

(71) Applicants: Charles Jeffrey Brinker, Albuquerque, NM (US); Eric C. Carnes, Albuquerque, NM (US); Carlee Erin Ashley, Albuquerque, NM (US); Joshua Santarpia, Albuquerque, NM (US); Adrienne Celeste Greene, Albuquerque, NM (US); Oscar Negrete, Pleasanton, CA (US); Steven Branda, Livermore, CA (US); Ayse Muniz, Albuquerque, NM (US); Jacob Ongundi Agola, Albuquerque, NM (US); Kimberly Butler, Albuquerque, NM (US)

(72) Inventors: Charles Jeffrey Brinker, Albuquerque, NM (US); Eric C. Carnes, Albuquerque, NM (US); Carlee Erin Ashley, Albuquerque, NM (US); Joshua Santarpia, Albuquerque, NM (US); Adrienne Celeste Greene, Albuquerque, NM (US); Oscar Negrete, Pleasanton, CA (US); Steven Branda, Livermore, CA (US); Ayse Muniz, Albuquerque, NM (US); Jacob Ongundi Agola, Albuquerque, NM (US); Kimberly Butler, Albuquerque, NM (US)

(21) Appl. No.: 15/474,810

(22) Filed: Mar. 30, 2017

Related U.S. Application Data
(63) Continuation-in-part of application No. PCT/US2015/053244, filed on Sep. 30, 2015.
(60) Provisional application No. 62/057,968, filed on Sep. 30, 2014.
(60) Provisional application No. 62/129,028, filed on Mar. 5, 2015.

(51) Int. Cl. A61K 9/127; C12N 9/22; A61K 9/51; A61K 49/00

(52) U.S. Cl. CPC A61K 9/1274; C12N 9/22; A61K 9/5123; C12N 2310/11; A61K 49/005; C12N 2310/20; A61K 9/5115

(57) ABSTRACT

The present disclosure relates to the delivery of polynucleotides and/or oligonucleotides using silica delivery platforms, e.g., silica carriers or protocells. In particular, in the present disclosure, polynucleotides in the form of plasmids expressing siRNA may be administered as cargo in the silica delivery platform to a patient or subject to inhibit and/or treat cancer in a patient. In one aspect, the silica delivery platform that have been charged with cargo comprising plasmid DNA (in particular, CRISPR ds plasmid DNA) which expresses siRNA, shRNA, mRNA and other RNA which may be used to administer these plasmids to patients in order to effect inhibition of cancer cells (especially including apoptosis of those cancer cells) and effective and/or prophylaxis of cancer, as well as numerous pathogens, including viruses, bacteria, fungi, and/or other disease states and/or conditions. In another aspect, the silica delivery platform comprises a biological package (e.g., plasmid nucleic acid, such as a for a CRISPR/Cas system) that interacts with a genomic sequence to either activate or inhibit gene expression. Such vehicles can be employed to control gene activation and repression in a host (e.g., a patient) and/or a pathogen.

### CROSS-REFERENCE TO RELATED APPLICATIONS

This application is a continuation-in-part of International Application No. PCT/US15/53244, filed Sep. 30, 2015, which in turn claims the benefit of U.S. Provisional Application No. 62/057,968, filed Sep. 30, 2014, and U.S. Provisional Application No. 62/129,028, filed Mar. 5, 2015, each of which is hereby incorporated by reference in its entirety.

### GRANT SUPPORT

This invention was made with government support under DE-AC04-94AL85000, awarded by the Department of Energy, U01 CA151792, awarded by the National Cancer Institute and EY016570, awarded by the National Institutes of Health. The government has certain rights in the invention.

### BACKGROUND

Over the past two years, CRISPR/Cas systems have been used to ‘perform genetic microsurgery’ on mice, rats, bacteria, yeast, plants, and human cells, thereby triggering a biotechnology revolution that has resulted in over 125 published manuscripts and in CRISPR being named Science magazine's ‘2013 Breakthrough of the Year’ runner-up.11, 12 In order to easily manipulate genes using CRISPR, researchers fused naturally-occurring tracrRNA and crRNA into a single, synthetic ‘guide RNA’ that directs Cas9 to virtually any desired DNA sequence (see FIG. 9); Additionally, synthetic CRISPR/Cas9 systems have sufficient selectivity for target DNA sequences to enable development of both pathogen- and host-directed countermeasures; this dual-pronged approach promises to kill target pathogens and interrupt critical pathogen-host interactions (e.g. pathogen binding and internalization by host cells), thereby dramatically reducing the likelihood that pathogens will evolve resistance. Additionally, synthetic CRISPR/Cas9 systems have sufficient selectivity for target DNA sequences to enable development of both pathogen- and host-directed countermeasures; this dual-pronged approach promises to kill target pathogens and interrupt critical pathogen-host interactions (e.g. pathogen binding and internalization by host cells), thereby dramatically reducing the likelihood that pathogens will evolve resistance.

### SUMMARY

The present disclosure relates to the delivery of polynucleotides, oligonucleotides, and/or polynucleotides using a delivery platform, e.g., MSNPs, protocells, or silica carriers, as described herein.

In one particular embodiment of the present disclosure, polynucleotides in the form of plasmids expressing siRNA may be administered as cargo in a delivery platform (e.g., a protocell or a carrier) to a patient or subject to inhibit and/or treat cancer in a patient. In the present disclosure, protocells or carriers which have been charged with cargo comprising plasmid DNA (CRISPR plasmids) which express siRNA, shRNA, and other RNA which may be used to administer these plasmids to patients in order to effect inhibition of cancer cells (especially including apoptosis of those cancer cells) and effective and/or prophylaxis of cancer, as well as numerous pathogens, including viruses, bacteria, fungi, etc.

In particular embodiments of the disclosure, the approach to cancer and/or bacterial and/or viral treatment relies on CRISPR, which is a new gene editing approach that has been studied by using standard transfection agents that are not useful for in vivo applications. Pursuant to the present disclosure, in particular embodiments, the present disclosure relates to delivering CRISPR components by packaging them with the delivery platform herein and administering the protocells or carriers to the cancer patient. The data produced shows from the green fluorescent protein expression by HeLa cells to which the protocells or carriers were delivered CRISPR and that it is active. The CRISPR components are added as ds-plasmid DNA, thus allowing siRNA and other anticancer agents to be expressed from the ds-plasmid DNA, resulting in cancer therapy.

In yet other embodiments of the disclosure, the present disclosure relates to a delivery platform that can be used to genetically modify a target (e.g., a target sequence present in a genomic sequence of a host, such as a human host, or a pathogen, such as any described herein). In one instance, the delivery platform includes a CRISPR/Cas system (e.g., a type II CRISPR/Cas system, as well as modified versions thereof, such as a CRISPR/dCas9 system).

The delivery platform can be a protocell or a carrier (e.g., a silica carrier). In general, the protocell includes a nanoparticle core, a supported lipid layer, and a cargo (e.g., a CRISPR/Cas system) encapsulated within the core (e.g., within one or more pores defined within the core). In addition, the carrier (e.g., a silica carrier) includes a biological package, a silica shell encapsulating the package, an optional supported lipid layer, and an optional cargo (e.g., within one or more pores defined within the shell, if the shell is porous; and/or in proximity to an inner surface of the silica shell, e.g., complexed with the biological package with a covalent or non-covalent bond). Each element of the protocell or the carrier can be modified to include one or more components that facilitate specific targeting and effective delivery of the cargo or the package.

The delivery platform can be delivered to any useful target, including a host (e.g., a human subject) and/or non-host (e.g., a pathogen). The delivery platform can be used to delivery one or more cargos or biological packages, e.g., a CRISPR/Cas system and one or more other agents, such as a drug (e.g., one or more antiviral agents, antimicrobial agents, antibacterial agents, etc.). Additional details follow.

### BRIEF DESCRIPTION OF THE FIGURES

FIG. 1 shows PEG/PEI MSNP on HEK 293 cells (GFP+ cells are green/light) all other cells stained blue. Nanoparticles are also shown. Also shown are the LipofectAmine® 2000 with CRISPR plasmids and GFP reporter.

FIG. 2 shows cell population expressing reporter gene in three instances 48 hours after transfection (3.75%) of HeLa cells.

FIG. 3 shows HeLa 48 hours after 0.5% loading with Torus-shaped MSNP and DOTAP protocell.

FIG. 4 shows HeLa 48 hours after 0.5% loading with 8 nm pore MSNP and DOTAP protocell.

FIG. 5 shows HeLa 48 hours after 0.5% loading with 18 nm pore MSNP and DOTAP protocell.

FIG. 6 shows that HeLa Cells- were silenced using siRNA delivered using 8 nm pore MSNP/DOTAP Protocell with siRNA.

FIG. 7 shows confirmation of knockdown (GFP) using MSNP/DOTAP protocell compared to a 72% knockdown using LipofectAmine® 2000.

FIG. 8 shows CRISPR plasmid delivery to HEK 293 using PEG/PEI MSNP-GFP Reporter.

FIG. 9 shows that CRISPR plasmid technology is a revolutionary/disruptive technology for gene editing—fast, easy, and cheap—requiring a new ‘scrap’ of RNA. By designing guide RNA sequences against any existing or hypothetical pathogen, we will design CRISPR components to completely annihilate bacterial function. Knockout multiple orthogonal conserved genetic pathways to avoid resistance and this approach can produce effective treatments in days rather than months.

FIG. 10A-10C shows a CRISPR component and its non-limiting use with a delivery platform described herein. (A) CRISPR naturally evolved in prokaryotes as a type of acquired immune system, conferring resistance to exogenous genetic sequences introduced by plasmids and phages. The CRISPR array is a noncoding RNA transcript, and the CRISPR repeat arrays are often associated with Cas (i.e., ‘CRISPR-associated’) protein families Exogenous DNA is cleaved by Cas proteins into ˜30-bp fragments, which are then inserted into the CRISPR locus (see (1) Acquisition in FIG. 10A, left). RNAs from the CRISPR loci are constitutively expressed (see (2) Expression in FIG. 10A, right) and direct other Cas proteins to cleave exogenous genetic elements upon subsequent exposure or infection (see (3) Interference in FIG. 10A, right). Cas9 is a RNA-Guided Endonuclease (R-GEN) adapted from the prokaryotic CRISPR system and is used by researchers as a novel, programmable tool for genome editing. Cas9 forms a sequence-specific endonuclease when complexed with a guide RNA that is complementary to the target sequence. (B) An exemplary CRISPR component includes a guiding component 90 to bind to the target sequence 97, as well as a nuclease 98 (e.g., a Cas nuclease or an endonuclease, such as a Cas endonuclease) that interacts with the guiding component and the target sequence. (C) The schematic depicts a delivery platform (e.g., a NanoCRISPR having a silica carrier) that interacts with host cells in order to achieve intracellular delivery of CRISPR-based therapeutics. (1) Targeting ligands conjugated to the NanoCRISPR surface can bind to corresponding receptors on the host cell. (2) Binding can trigger receptor-mediated endocytosis of NanoCRISPRs. (3) Endosomes become acidified, which will cause the lipid coating to dissociate from the NanoCRISPR's silica surface. (4) Endosome acidification will also protonate endosomolytic peptides, which will rupture endosomes via the proton-sponge mechanism. (5) Once in the cell's cytosol, the NanoCRISPR's silica shell will dissolve via hydrolysis, thereby releasing encapsulated CRISPR/Cas9 constructs (plasmids, in this case) and allowing them to act on their target RNA or DNA sequence.

FIG. 11A-11C shows exemplary silica carriers. Provided are (A) a silica carrier 105 formed around a biological package 101 having a dimension d_{b} and (B) a silica carrier 1005 formed around a biological package 1001 and further including one or more cargos 1006. (C) Also provided is a schematic depicting use of a silica carrier as a NanoCRISPR platform to deliver CRISPR components in a targeted manner The left half of the schematic depicts the NanoCRISPR(s) for a virus (e.g., an Ebola virus (EBOV)), and the right half depicts the NanoCRISPR(s) for a bacterium (e.g., Burkholderia pseudomallei (Bp)). NanoCRISPRs can include a therapeutic biological package (e.g., plasmids that encode Cas/guiding components that target the viral RNA genome; and bacteriophages that infect bacterium and encode Cas/guiding components that target essential bacterial genes in the bacterial DNA genome) coated with a shell of amorphous silica to stabilize the therapeutic, both upon room-temperature storage and in the bloodstream, and control its rate of release inside of target host cells. The silica surface can be optionally modified with biocompatible lipids to increase the colloidal stability of NanoCRISPRs and to facilitate their conjugation with ligands that target organs and cells that the particular virus and/or bacterium (e.g., EBOV and Bp) infect or that promote endosomal escape of NanoCRISPRs upon host cell uptake.

FIG. 12A-12B shows exemplary protocells. Provided are (A) a protocell 205 having a porous core 201 having a dimension d_{core} and a dimension dₚₒᵣₑ and (B) a schematic depicting use of a protocell as a NanoCRISPR platform for highly efficacious delivery of CRISPR-based medical countermeasures. Pathogen-directed and host-directed CRISPR components (e.g., guide components, such as guide RNAs, as well as minicircle DNA vectors that encode Cas and guiding components) will be developed, along with strategies for introducing CRISPR components into pathogenic bacteria. Non-limiting strategies include modifying CRISPR components will cell-penetrating peptides, co-delivering CRISPR components with metal organic frameworks (MOFs) designed to permeabilize bacteria, and/or developing phage that encode CRISPR components. CRISPR components can be loaded within mesoporous silica nanoparticles (MSNPs) and/or encased in a supported lipid bilayer (SLB). Resulting NanoCRISPRs can be optionally surface-modified with molecules that promote their accumulation with infected organs and trigger their uptake by infected host cells.

FIG. 13 shows a schematic of a NanoCRISPR delivery platform (e.g., a protocell or a silica carrier) interacting with an infected host cell to deliver pathogen-directed and host-directed CRISPR-based medical countermeasures. While small molecule antimicrobials were omitted from this schematic, the NanoCRISPR platform can simultaneously encapsulate and deliver complex combinations of CRISPR components, as well as any other useful agent (e.g., antiviral agents, antibacterial agents, anticancer agents, labels, reporters, siRNAs, as well as any other agent described herein). Although particular pathogens are provided, i.e., a virus (Vaccinia virus) and a bacterium (B. pseudomallei), any useful pathogen can be targeted using the delivery platforms described herein.

FIG. 14 shows a schematic of non-limiting ways to combining CRISPR and the delivery platforms (or delivery technologies) described herein. As can be seen, the combination creates a modular, generic strategy for rapidly designing and formulating medical countermeasures against viral and bacterial pathogens. Delivery platforms that are optimized for encapsulation of various cargo molecules or biological packages, as well as targeted accumulation within various organ and cellular targets can be synthesized and stockpiled. Likewise, CRISPR components that target sequences in pathogens that will likely serve as ‘chasses’ for genetically-enhanced agents can be designed, tested for in vitro efficacy and safety, and pre-produced. CRISPR components and delivery systems can then be combined to rapidly generate new medical countermeasures suitable for prophylaxis and treatment. Cargo molecules, as well as organ, cellular, and molecule targets, can be tested.

FIG. 15 shows a schematic of the CRISPR-Cas9 nuclease heterocomplex. As can be seen, one non-limiting CRISPR component includes a guiding component, which in turn is a single, nucleic acid sequence having a targeting portion and an interacting portion. The targeting portion can include (1) a nucleic acid sequence that imparts specific targeting to the target genomic locus. The interacting portion can include (2) a short crRNA sequence attached to the targeting portion; and (3) a tracrRNA sequence attached to the crRNA sequence, where the chimeric crRNA-tracrRNA sequence facilitates recruitment of the Cas9 nuclease, which cleaves the genomic target.

FIG. 16A-16H shows non-limiting amino acid sequences for various nucleases. Provided are sequences for (A) a Cas9 endonuclease for S. pyogenes serotype Ml (SEQ ID NO:110), (B) a deactivated Cas9 having D10A and H840A mutations (SEQ ID NO:111), (C) a Cas protein Csn1 for S. pyogenes (SEQ ID NO:112), (D) a Cas9 endonuclease for F. novicida U112 (SEQ ID NO:113), (E) a Cas9 endonuclease for S. thermophilus 1 (SEQ ID NO:114), (F) a Cas9 endonuclease for S. thermophilus 2 (SEQ ID NO:115), (G) a Cas9 endonuclease for L. innocua (SEQ ID NO:116), and (H) a Cas9 endonuclease for W. succinogenes (SEQ ID NO:117).

FIG. 17A-17C shows non-limiting CRISPR components. Provided are schematics of (A) a non-limiting guiding component 300 having a targeting portion 304, a first portion 301, a second portion 302, and a linker 303 disposed between the first and second portions; (B) another non-limiting guiding component 350 having a targeting portion 354, a first portion 351, a second portion 352 having a hairpin, and a linker 353 disposed between the first and second portions; and (C) non-limiting interactions between the guiding component 400, the genomic sequence 412, and the first and second portion 401,402. As can be seen, the target sequence 411 of the genomic sequence 412 is targeted by way of non-covalent binding 421 to the targeting portion 404, and secondary structure can be optionally implemented by way of non-covalent binding 422 between the first portion 401 and the second portion 402. The targeting portion 404, first portion 401, linker 403, and second portion 402 can be attached in any useful manner (e.g., to provide a 5′ end 405 and a 3′ end 406).

FIG. 18 shows non-limiting nucleic acid sequences of crRNA that can be employed as a first portion in any guiding component described herein. Provided are sequences for S. pyogenes (SEQ ID NO:20), L. innocua (SEQ ID NO:21), S. thermophilus 1 (SEQ ID NO:22), S. thermophilus 2 (SEQ ID NO:23), F. novicida (SEQ ID NO:24), and W. succinogenes (SEQ ID NO:25). Also provided are various consensus sequences (SEQ ID NOs:26-32), in which each X, independently, can be absent, A, C, T, G, or U, as well as modified forms thereof (e.g., as described herein). In another embodiment, for each consensus sequence (SEQ ID NOs:26-32), each X at each position is a nucleic acid (or a modified form thereof) that is provided in an aligned reference sequence. For instance, for consensus SEQ ID NO:26, the first position includes an X, and this X can be absent or any nucleic acid (e.g., A, C, T, G, or U, as well as modified forms thereof). Alternatively, this X can be any nucleic acid provided in an aligned reference sequence (e.g., aligned reference sequences SEQ ID NO:20-25 for the consensus sequence in SEQ ID NO:26). Thus, X at position 1 in SEQ ID NO:26 can also be G (as in SEQ ID NOs:20-23 and 25) or C (as in SEQ ID NO:24), in which this subset of substitutions is defined as a conservative subset. Similarly, for each X at each position for the consensus sequences (SEQ ID NOs:26-32), conservative subsets can be determined based on FIG. 18, and these consensus sequences include nucleic acid sequences encompassed by such conservative subsets. Gray highlight indicates a conserved nucleic acid, and the dash indicates an absent nucleic acid.

FIG. 19A-19C shows non-limiting nucleic acid sequences of tracrRNA that can be employed as a second portion and/or linker in any guiding component described herein. Provided are sequences for S. pyogenes (SEQ ID NO:40), L. innocua (SEQ ID NO:41), S. thermophilus 1 (SEQ ID NO:42), S. thermophilus 2 (SEQ ID NO:43), F. novicida 1 (SEQ ID NO:44), F. novicida 2 (SEQ ID NO:45), W. succinogenes 1 (SEQ ID NO:46), and W. succinogenes 2 (SEQ ID NO:47). Also provided are various consensus sequences (SEQ ID NOs:48-54), in which each Z, independently, can be absent, A, C, T, G, or U, as well as modified forms thereof (e.g., as described herein). Consensus sequences are shown for (A) an alignment of all SEQ ID NOs:40-47, providing consensus sequences SEQ ID NOs:48-50; (B) an alignment of SEQ ID NOs:40-43, providing consensus sequences SEQ ID NOs:51-52; and (C) an alignment of SEQ ID NOs:44-47, providing consensus sequences SEQ ID NOs:53-54. In another embodiment, for each consensus sequence (SEQ ID NOs:48-54), each Z at each position is a nucleic acid (or a modified form thereof) that is provided in an aligned reference sequence. For instance, for consensus SEQ ID NO:48, the first position includes a Z, and this Z can be absent or any nucleic acid (e.g., A, C, T, G, or U, as well as modified forms thereof). Alternatively, this Z can be any nucleic acid provided in an aligned reference sequence (e.g., aligned reference sequences SEQ ID NO:40-47 for the consensus sequence in SEQ ID NO:48). Thus, Z at position 2 in SEQ ID NO:48 can also be U (as in SEQ ID NOs:40, 41, and 43-47) or G (as in SEQ ID NO:42), in which this subset of substitutions is defined as a conservative subset. Similarly, for each Z at each position for the consensus sequences (SEQ ID NOs:48-54), conservative subsets can be determined based on FIG. 19A-19C, and these consensus sequences include nucleic acid sequences encompassed by such conservative subsets. Gray highlight indicates a conserved nucleic acid, and the dash indicates an absent nucleic acid.

FIG. 20 shows non-limiting nucleic acid sequences of extended tracrRNA that can be employed as a second portion and/or linker in any guiding component described herein. Provided are sequences for S. pyogenes (SEQ ID NO:60), L. innocua (SEQ ID NO:61), S. thermophilus 1 (SEQ ID NO:62), and S. thermophilus 2 (SEQ ID NO:63). Also provided are various consensus sequences (SEQ ID NOs:64-65), in which each Z, independently, can be absent, A, C, T, G, or U, as well as modified forms thereof (e.g., as described herein). In another embodiment, for each consensus sequence (SEQ ID NOs:64-65), each Z at each position is a nucleic acid (or a modified form thereof) that is provided in an aligned reference sequence. For instance, for consensus SEQ ID NO:64, the first position includes a Z, and this Z can be absent or any nucleic acid (e.g., A, C, T, G, or U, as well as modified forms thereof). Alternatively, this Z can be any nucleic acid provided in an aligned reference sequence (e.g., aligned reference sequences SEQ ID NO:60-63 for the consensus sequence in SEQ ID NO:64). Thus, Z at position 1 in SEQ ID NO:64 can also be absent (as in SEQ ID NO:60), A (as in SEQ ID NO:61), or U (as in SEQ ID NOs:63-64), in which this subset of substitutions is defined as a conservative subset. Similarly, for each Z at each position for the consensus sequences (SEQ ID NOs:64-65), conservative subsets can be determined based on FIG. 20, and these consensus sequences include nucleic acid sequences encompassed by such conservative subsets. Gray highlight indicates a conserved nucleic acid, and the dash indicates an absent nucleic acid.

FIG. 21 shows non-limiting nucleic acid sequences of a guiding component (e.g., a synthetic, non-naturally occurring guiding component) having a generic structure of A-L-B, in which A includes a first portion (e.g., any one of SEQ ID NOs:20-32, or a fragment thereof), L is a linker (e.g., a covalent bond, a nucleic acid sequence, a fragment of any one of SEQ ID NOs:40-54 and 60-65, or any other useful linker), and B is a second portion (e.g., any one of SEQ ID NOs:40-54 and 60-65, or a fragment thereof). Also provided are various embodiments of single-stranded guiding components (SEQ ID NOs:80-93). Exemplary non-limiting guiding components include SEQ ID NO:81, or a fragment thereof, where X at each position is defined as in SEQ ID NO:26 and Z at each position is as defined in SEQ ID NO:48; SEQ ID NO:82, or a fragment thereof, where X at each position is defined as in SEQ ID NO:27 and Z at each position is as defined in SEQ ID NO:49; SEQ ID NO:83, where X at each position is defined as in SEQ ID NO:28 and Z at each position is as defined in SEQ ID NO:49; SEQ ID NO:84, or a fragment thereof, where X at each position is defined as in SEQ ID NO:27 and Z at each position is as defined in SEQ ID NO:65; SEQ ID NO:85, or a fragment thereof, where X at each position is defined as in SEQ ID NO:28 and Z at each position is as defined in SEQ ID NO:65; SEQ ID NO:86, or a fragment thereof, where X at each position is defined as in SEQ ID NO:29 and Z at each position is defined as in SEQ ID NO:51; SEQ ID NO:87, or a fragment thereof, where X at each position is defined as in SEQ ID NO:30 and Z at each position is defined as in SEQ ID NO:51; SEQ ID NO:88, or a fragment thereof, where X at each position is defined as in SEQ ID NO:30 and Z at each position is defined as in SEQ ID NO:52; SEQ ID NO:89, or a fragment thereof, where X at each position is defined as in SEQ ID NO:30 and Z at each position is defined as in SEQ ID NO:65; SEQ ID NO:90, or a fragment thereof, where X at each position is defined as in SEQ ID NO:31 and Z at each position is defined as in SEQ ID NO:51; SEQ ID NO:91, or a fragment thereof, where X at each position is defined as in SEQ ID NO:32 and Z at each position is as defined in SEQ ID NO:53; SEQ ID NO:92, or a fragment thereof, where X at each position is defined as in SEQ ID NO:32 and Z at each position is as defined in SEQ ID NO:54; and SEQ ID NO:93, or a fragment thereof, where X at each position is defined as in SEQ ID NO:32 and Z at each position is defined as in SEQ ID NO:65. The fragment can include any useful number of nucleotides (e.g., any number of contiguous nucleotides, such as a fragment including about 4, 5, 6, 7, 8, 9, 10, 11, 12, 15, 18, 20, or more contiguous nucleotides of any sequences described herein, such as a sequence for the first portion, e.g., any one of SEQ ID NOs:20-32; and also such as a fragment including about 4, 5, 6, 7, 8, 9, 10, 11, 12, 15, 18, 20, 24, 26, 28, 30, 32, 34, 38, 36, 40, or more contiguous nucleotides of any sequences described herein, such as a sequence for the first portion, e.g., any one of SEQ ID NOs:40-54 and 60-65).

FIG. 22 shows additional non-limiting nucleic acid sequences of a guiding component (e.g., a synthetic, non-naturally occurring guiding component). Provided are various embodiments of single-stranded guiding components (SEQ ID NOs:100-103). Exemplary non-limiting guiding components include SEQ ID NO:100, or a fragment thereof, where n at each of positions 1-80 can be present or absent such that this region can contain anywhere from 12 to 80 nucleotides and n is A, C, T, G, U, or modified forms thereof; and where n at each of positions 93-192 can be present or absent such that this region can contain anywhere from 3 to 100 nucleotides and n is A, C, T, G, U, or modified forms thereof; SEQ ID NO:101, or a fragment thereof, where n at each of positions 1-80 can be present or absent such that this region can contain anywhere from 12 to 80 nucleotides and n is A, C, T, G, U, or modified forms thereof; and where n at each of positions 93-192 can be present or absent such that this region can contain anywhere from 3 to 100 nucleotides and n is A, C, T, G, U, or modified forms thereof; SEQ ID NO:102, or a fragment thereof, where n at each of positions 1-80 can be present or absent such that this region can contain anywhere from 12 to 80 nucleotides and n is A, C, T, G, U, or modified forms thereof; and SEQ ID NO:103, or a fragment thereof, where n at each of positions 1-80 can be present or absent such that this region can contain anywhere from 12 to 80 nucleotides and n is A, C, T, G, U, or modified forms thereof.

FIG. 23 shows an aerosol-assisted EISA for a rapid, cost-effective, scalable method for producing MSNPs with reproducible properties. Provided are (A) a non-limiting schematic and (B) a photograph of an exemplary reactor to generate MSNPs, protocells, and/or carriers via aerosol-assisted EISA. Numbers indicate corresponding portions of the reactor.

FIG. 24 shows that aerosol-assisted EISA can be used to generate MSNPs with various pore geometries. TEM images of MSNPs with hexagonal (A), cubic (B), lamellar (C), and cellular (D-E) pore geometries (F) shows dual-templated particles with interconnected 2 nm and 60 nm pores. Light grey/white areas are voids (i.e., pores), while dark grey/black areas are silica. See Lu, Brinker, et al. Nature (1999) for further details.

FIG. 25 shows that aerosol-assisted EISA can be used to generate MSNPs with various pore sizes. TEM images of MSNPs with 2.5 nm pores templated by CTAB (A), 4.4 nm pores templated by F68 (B), 7.9 nm pores templated by F127 (C), and 18-25 nm pores templated by crosslinked micelles (D). The inset in (D) is a SEM micrograph that shows the presence of surface-accessible pores.

FIG. 26 shows that lipid coated silica (LCS) delivery platforms have extremely high loading capacities for various antibiotics. Molecular weights (MW) and net charges at physiological pH are given for each antibiotic. Data represent the mean+std. dev. for n=3

FIG. 27 shows the degree of condensation of the MSNP core, which can be used to tailor release rates from burst to sustained profiles. Rates of gentamicin release from MSNPS with a low (A) and high (B) degree of silica condensation. Silica forms via a condensation reaction (C) and dissolves via a hydrolysis reaction (D); the degree of silica condensation dictates that number of Si—O—Si bonds that must be broken for the particle to dissolve and can, therefore, be used to control release rates. Data represent the mean±std. dev. for n=3.

FIG. 28 shows that lipid coated silica (LCS) delivery platforms (or LCS particles) that are targeted to Bp host cells dramatically improve the in vitro efficacy of gentamicin, an antibiotic to which many strains of Bp are resistant. (A)-(B) Dose (A) and time (B) response curves for free ceftazidime, free gentamicin, ceftazidime loaded in Fcγ-targeted LCS platforms, and gentamicin loaded in Fcγ-targeted LCS platforms. In (A), infected THP-1 cells were then incubated with various concentrations of ceftazidime or gentamicin samples for 24 hours. In (B), infected THP-1 cells were incubated for various periods of time with 5 μg/mL of ceftazidime samples or 100 μg/mL of gentamicin samples. Cells were vortexed with glass beads to release intracellular Bp, and Bp CFUs were determined by plating on LB agar. Data represent the mean±std. dev. for n=3.

FIG. 29 shows LCS delivery platforms that are targeted to the lung or liver and spleen dramatically increase the in vivo efficacy of gentamicin in mice challenged with a lethal dose of gentamicin-resistant Bp. (A)-(B) Bacterial burden (A) and survival (B) of BALB/c mice upon intranasal challenge with 500 CFUs of Bp; mice were treated 24 hours after infection via IV injection with 20 mg/kg of free gentamicin, 20 mg/kg of gentamicin loaded in non-targeted LCS delivery platforms, or 20 mg/kg of gentamicin loaded in targeted LCS delivery platforms; mice that received no treatment or empty LCS delivery platforms were included as controls. For (A) bacterial burdens were measured upon euthanasia, and data represent the mean±std. dev. for 10 mice. LCS delivery platforms were targeted to the lung using a peptide ‘zip-code’ that binds to lung vasculature and to the liver and spleen using mannosylated cholesterol.

FIG. 30 shows that LCS delivery platforms are selectively internalized by model Bp host cells when modified with cell-specific targeting ligands. (A) The number of LCS particles internalized by THP-1 (model macrophage), A549 (model alveolar epithelial cell), and HepG2 (model hepatocyte) cells upon incubation with a 10⁴-fold excess of LCS particles for 1 hour at 37° C. LCS particles were coated with DOPC (net neutral charge at physiological pH), DOPS (net negative charge), or DOTAP (net positive charge); DOPC LCS particles were further targeted to THP-1, A549, and HepG2 cells using a DEC-205 scFv, the GE11 peptide, and the SP94 peptide, respectively. Data represent the mean±std. dev. for n=3. (B) Confocal fluorescence microscopy images of THP-1, A549, and HepG2 cells after being incubated with a 10⁴-fold excess of LCS particles for 1 hour at 37° C. LCS particles were loaded with pHrodo Red (red), the fluorescence intensity of which dramatically increases under endolysosomal conditions, and labeled with NBD (green), the fluorescence intensity of which is independent of pH, and targeted to THP-1, A549, and HepG2 cells using a DEC-205 scFv, the GE11 peptide, and the SP94 peptide, respectively. Cell nuclei were stained with DAPI (blue).

FIG. 31 shows that protocells have high capacities for physicochemically disparate medical countermeasures and controllable, pH-triggered release rates. (A) Loading capacities of 150 nm protocells with 2.5 nm pores, 4.4 nm pores, 7.9 nm pores, and 18-25 nm pores for different classes of small molecule (ribavirin, ceftazidime), protein (hPON-1, OPH, hBuChE), and nucleic acid (siRNA, mcDNA, pDNA)-based medical countermeasures (siRNA, mcDNA, pDNA); loading capacities of 150 nm liposomes are provided for comparison. Molecular weights (MW) and mean hydrodynamic sizes in 1× PBS are given for each cargo molecule. * indicates the hydrodynamic size of the pDNA after being packaged with histones. (B) Rates of ribavirin release from protocells with DOPC SLBs when incubated in a simulated body fluid (pH 7.4) or a simulated endolysosomal fluid (pH 5.0) at 37° C. for 7 days; the rate of ribavirin release from DSPC liposomes upon incubation in a simulated body fluid is given for comparison. Data represent the mean±std. dev. for n=3.

FIG. 32 shows that size controls the bulk biodistribution of non-targeted LCS delivery platforms. Total mass of silica (SiO₂) in the blood, liver, spleen, lymph nodes, kidneys, bladder, lungs, heart, brain, urine, and feces of Balb/c mice 1 day, 1 week, and 1 month after being injected IV with 200 mg/kg (˜5 mg of SiO₂ per mouse) of 150 nm or 250 nm DOPC LCs particles. Each bar represents the mean+std. dev. for 2 mice. ND=none detected.

FIG. 33 shows that surface modifications can overwhelm size-dependent biodistribution for 150 nm LCS particles. Total mass of silica (SiO₂) in the blood, liver, spleen, lymph nodes, kidneys, bladder, lungs, heart, brain, urine, and feces of Balb/c mice that were injected IV with 200 mg/kg (˜5 mg of SiO₂ per mouse) of 150 nm DOPC LCS particles modified with CD47 (A) or with a proprietary antibody that targets the lungs (B). Each bar represents the mean+std. dev. for 2 mice. ND=none detected.

FIG. 34 shows that LCS particles remain stable in blood, as evidenced by their near-constant sizes and surface charges. Mean hydrodynamic size (A) and zeta potential (B) of LCS particles, LCS particles modified with 10 wt % of PEG-2000, PEI-coated silica NPs, PEI-coated silica NPs modified with 10 wt % of PEG-2000 upon incubation in whole blood for 7 days at 37° C. Data represent the mean±std. dev. for n=3.

FIG. 35 shows that spray-drying LCS particles increases their room-temperature shelf-life. Time-dependent release of gentamicin from DOPC LCS particles that were stored in 1× PBS, as well as DOPC LCS particles that were spray-dried in the presence of trehalose or poly(lactide-co-glycolide) (PLGA) and stored in nitrogen-flushed septum vials. Data represent the mean±std. dev. for n=3.

FIG. 36 shows that the supported lipid layers enabled pH-triggered release, where cargo molecules are retained in blood but released in a simulated endolysosomal fluid at various rates. (A)-(B) Rates of gentamicin release from DOPC LCS particles when incubated in blood or a simulated endolysosomal fluid (SEF) at 37° C. for 14 days or 72 hours, respectively. LCS particles had a low or high degree of condensation (DOC). Supported lipid bilayers (SLBs) were either unmodified or modified to contain 5 wt % of a maleimide-containing lipid (MPB) that forms disulfide bond-based crosslinks in the presence of DTT. Supported lipid multilayers (SLMs) were three layers thick. Data represent the mean±std. dev. for n=3.

FIG. 37 shows eight-color confocal fluorescence microscopy images of cells incubated with a 10⁴-fold excess of LCS particles for 1 hour (A) or 24 hours (B) at 37° C. LCS particles were simultaneously loaded with a fluorescently-labeled model drug (green), siRNA mimic (a dsDNA, magenta), protein (orange), and QD-conjugated minicircle DNA (cyan); the lipid (red) and silica (yellow) components of the LCS particle were individually labeled as well (figure adapted from Ashley et al., 2012). Cells were stained with CellTracker Violet BMQC (purple) and DAPI (blue).

FIG. 38 shows that by varying size and surface modifications, LCS particles can be engineered to rapidly accumulate in the spleen and liver. Time-dependent concentrations (depicted as percent of the injected dose, or % ID) of silicon (from silica NPs) and rhodamine B (used as a surrogate drug) in the spleens (A) and livers (B) of BALB/c mice upon IV injection of 50 mg/kg of free rhodamine B or rhodamine B loaded in LCS particles. LCS particles had a mean diameter of 320 nm with a 210-450 nm size distribution and were modified with Fcγ, a protein that targets innate immune cells; see FIG. 39 for spleen vs. liver accumulation for smaller LCS particles (30-100 nm) and for unmodified 210-450 nm LCS particles. Silicon and rhodamine B concentrations in the spleens and livers (collected from the same mice) were determined using ICP-MS and HPLC-FLD, respectively. Error bars represent the mean±the standard deviation for 5 mice.

FIG. 39 shows that the extent to which LCS particles accumulate in the liver vs. spleen is determined by their size and surface modifications. Time-dependent concentrations (depicted as percent of the injected dose, or % ID) of silicon (from silica NPs) in the livers and spleens of BALB/c mice upon IV injection of 50 mg/kg of DOPC LCS particles or DOPC LCS particles targeted with mannosylated cholesterol (MCh). In (A), LCS particles had a mean diameter of 70 nm with a 30-110 nm size distribution. In (B), LCS particles had a mean diameter of 320 nm with a 210-450 nm size distribution. Silicon concentrations were determined using ICP-MS. Error bars represent the mean±the standard deviation for 10 mice.

FIG. 40 shows that by varying size, surface modifications, and route of administration, LCS particles can be engineered to accumulate in the lungs. Time-dependent concentrations (depicted as percent of the administered dose, or % AD) of silicon (from silica NPs) and rhodamine B (used as a surrogate drug) in the lungs of BALB/c mice upon IV injection (A) or aerosolization (B) of 50 mg/kg of free rhodamine B or rhodamine B loaded in LCS particles. In (A), LCS particles had a mean diameter of 70 nm with a 30-110 nm size distribution and were modified with a peptide ‘zip-code’ that was identified via in vivo phage display to target lung vasculature; see FIG. 41 for lung vs. liver accumulation of zip-code-targeted LCS particles. In (B), LCS particles had a mean diameter of 200 nm with a 100-420 nm size distribution and were aerosolized using a PurRD jet nebulizer and administered to mice using a nose-only exposure chamber. Silicon and rhodamine B concentrations in the lungs were determined using ICP-MS and HPLC-FLD, respectively. Data represent the mean±the standard deviation for 5 mice.

FIG. 41 shows that LCS particles that are targeted to the lung preferentially accumulate in the lungs over the liver. Time-dependent concentrations (depicted as percent of the injected dose, or % ID) of silicon (from silica NPs) in the livers and lungs of BALB/c mice upon IV injection of 50 mg/kg of DOPC LCS particles or DOPC LCS particles modified with a peptide ‘zipcode’ that targets lung vasculature. LCS particles had a mean diameter of 70 nm with a 30-110 nm size distribution. Silicon concentrations were determined using ICP-MS. Error bars represent the mean±the standard deviation for 10 mice.

FIG. 42 shows that by varying size and surface modifications, LCS particles can be engineered to remain in circulation for long periods of time. Time-dependent concentrations (depicted as percent of the injected dose, or % ID) of silicon (from silica NPs) and rhodamine B (used as a surrogate drug) in the blood of BALB/c mice upon IV injection of 50 mg/kg of free rhodamine B or rhodamine B loaded in LCS particles. LCS particles had a mean diameter of 70 nm with a 30-110 nm size distribution and were modified with CD47, a protein expressed by red blood cells that innate immune cells recognize as ‘self’. Silicon and rhodamine B concentrations in whole blood were determined using ICP-OES and HPLC-FLD, respectively. Error bars represent the mean±the standard deviation for 5 mice.

FIG. 43 shows that LCS particles are biodegradable. Concentrations (depicted as percent of the injected dose, or % ID) of silicon (from silica NPs) in the urine and feces of BALB/c mice 1 hour, 24 hours, 48 hours, 72 hours, 7 days, and 14 days after IV injection of a 200 mg/kg dose of empty DOPC LCS particles (70 nm in diameter with 30-110 nm size distribution). Silicon was quantified using ICP-MS. Data represent the mean+std. dev. for 5 mice. ND=none detected.

FIG. 44 shows that LCS particles are non-immunogenic. Serum IgG and IgM titers induced upon SC immunization of C57B1/6 mice with three doses of LCS particles or albumin NPs that were targeted to hepatocytes with a peptide (‘SP94’) identified via phage display. Mice were immunized on days 0, 14, and 28 with 20 μg of LCS particles or albumin NPs; serum was collected on day 56, and peptide-specific IgG and IgM titers were determined via end-point dilution ELISA. Data represent the mean+std. dev. for 3 mice.

FIG. 45 shows that LCS particles that are engineered to accumulate in the lungs, liver, and spleen or to remain in circulation effectively treat gentamicin-resistant Bt infections in mice when administered up to 5 days before or 4 days after intranasal challenge. Summary of the sizes, surface modifications, and routes of administration we used to achieve 100% survival for 14 days or ≧80% survival for 7 days when 20 mg/kg of gentamicin-loaded LCS particles were administered to BALB/c mice at various time points before or after intranasal challenge with 1×10⁴ CFUs of Bt.

FIG. 46 shows that formulating a model phage, MS2, in silica carriers (e.g., single phage-in-silica nanoparticles or “SPS NPs”) increases its room-temperature shelf-life and decreases its immunogenicity. (A) Titers of a MS2 liquid stock, MS2 spray-dried in the presence of Brij 58 (2.5-μm mean diameter), MS2 spray-dried in the presence of sucrose (2.2-μm mean diameter), MS2-based SPS NPs that do not contain silica (93 nm mean diameter), MS2-based SPS NPs that do contain silica (55 nm mean diameter), and silica-containing SPS NPs that were further spray-dried in the presence of trehalose (2.5-μm mean diameter) upon storage for 6 months at ambient temperature and humidity. MS2 stored as a liquid stock loses 460 logs of activity per month. Spray-dried MS2 loses 19-26 logs of activity per month. SPS NPs formed without silica lose 5.9 logs of activity per month. SPS NPs formed with silica lose 0.37 logs of activity per month. Finally, spray-dried SPS NPs lose 0.21 logs of activity in six months. (B) Anti-MS2 serum IgG titers for free MS2, MS2 spray-dried (SD) in the presence of sucrose, and MS2-based SPS NPs that contain silica, Brij 58, and sucrose. C57B1/6 mice were immunized SC with 20 μg of MS2 on days 0, 14, and 28; serum was collected on day 56, and MS2-specific IgG titers were determined via end-point dilution ELISA. Each circle represents the titer achieved in one of four mice per group; lines represent the average titer per group.

FIG. 47 shows that spray-drying of silica carriers (e.g., SPS NPs) results in inhalable dry powders that show promising lung deposition upon insufflator-based administration to mice. (A)-(D) Size (A) and morphology (B-D) of dry powder particles (2.5 μm mean diameter) obtained upon spray-drying SPS NPs (55 nm mean diameter) in the presence of lactose. Size was determined using optical particle spectrometry, and morphology was determined using SEM (B, C) and TEM (D); arrows in (C) and (D) point to SPS NPs. SPS NPs contained the model phage, MS2, and were coated with the zwitterionic lipid, DOPC, prior to spray-drying; MS2 was labeled with electron-dense Sulfo-NHS-Nanogold® prior to its incorporation in SPS NPs. (E)-(F) The trachea, right lung, and left lung from BALB/c mice 1 hour after receiving no treatment (E) or 50 mg/kg of fluorescently-labeled SPS NPs in 200 μL puffs via a PennCentury dry powder insufflator, model DP-4 (F). The scale in (F) has units of (p/sec/cm²/sr)/(W/cm²).

FIGS. 48A-C. A549 reporter cell editing using protocell delivery of Cas9/gRNA complexes. A) A549 lung epithelial CRISPR reporter cells were treated with hexagonal MSNPs containing CRISPR RNPs and a DOTAP cationic lipid bilayer. GFP expression induced through gene editing was measured 72 hours after treatment with 25 μg of protocells in a 12-well plate of cells. RFP is constitutively expressed in these cells. B) GFP expression was measured using flow cytometry. Control conditions are displayed using grey bars. Blue wbars represent treatments of cell containing only MNSP and RNPs without lipid bilayers and green bars indicate gene editing in cells when treated with protocells using DOTAP lipid bilayers. C) Comparison of different formulations.

### DETAILED DESCRIPTION

The following terms shall be used throughout the specification to describe the present disclosure. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either both of those included limits are also included in the disclosure. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

It must be noted that as used herein and in the appended claims, the singular forms “a,” “and” and “the” include plural references unless the context clearly dictates otherwise.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present disclosure is provided. For treatment of those infections, conditions or disease states which are specific for a specific animal such as a human patient, the term patient refers to that specific animal. In most instances, the patient or subject of the present disclosure is a human patient of either or both genders.

The term “effective” is used herein, unless otherwise indicated, to describe an amount of a compound, composition or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diastereomers), individual optical isomers (enantiomers) or racemic mixtures, pharmaceutically acceptable salts (including alternative pharmaceutically acceptable salts when a pharmaceutically acceptable salt is disclosed) and prodrug forms. The term compound herein refers to stable compounds. Within its use in context, the term compound may refer to a single compound or a mixture of compounds as otherwise described herein. One or more bioactive agent (any agent which produces an intended biological, including pharmacological effect) may be included in MSNPs according to the present disclosure to provide pharmaceutical compositions hereunder and preferably the bioactive agent is (double stranded) ds plasmid DNA which expresses RNA, including siRNA, shRNA or mRNA often and preferably from a CRISPR plasmid delivered as cargo in a protocell or a silica carrier.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts, pharmaceutically acceptable salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth). The salts can be prepared in situ during the final isolation and purification of the compounds of the disclosure or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine.

The term “mesoporous silica nanoparticles” (MSNPs) includes nanoparticles according to the present disclosure which are modified to target specific cells (in many instances, cancer cells) in vivo for diagnostic and/or therapeutic purposes. Particularly relevant MSNPs for use in the present disclosure are described in international patent application PCT/US2014/56312, filed Sep. 18, 2014, entitled “Core and Surface Modification of Mesoporous Silica Nanoparticles to Achieve Cell Specific Targeting in Vivo”, and application PCT/US2014/56342, also filed Sep. 18, 2014, entitled “Torroidal Mesoporous Silica Nanoparticles (MSNPs) and Related Protocells”, both of which applications are incorporated herein in their entirety.

A particle or a portion thereof (e.g., a protocell, a carrier, a core of the protocell, a shell of the carrier, etc.) may have a variety of shapes and cross-sectional geometries that may depend, in part, upon the process used to produce the particles. The particle can be a nanoparticle (e.g., having a diameter less than about 1 μm) or a microparticle (e.g., having a diameter greater than or equal to about 1 μm). In one embodiment, a particle may have a shape that is a sphere, a donut (torroidal), a rod, a tube, a flake, a fiber, a plate, a wire, a cube, or a whisker. A particle may include particles having two or more of the aforementioned shapes. In one embodiment, a cross-sectional geometry of the particle may be one or more of circular, ellipsoidal, triangular, rectangular, or polygonal. In one embodiment, a particle may consist essentially of non-spherical particles. For example, such particles may have the form of ellipsoids, which may have all three principal axes of differing lengths, or may be oblate or prelate ellipsoids of revolution. Non-spherical particles alternatively may be laminar in form, wherein laminar refers to particles in which the maximum dimension along one axis is substantially less than the maximum dimension along each of the other two axes. Non-spherical particles may also have the shape of frusta of pyramids or cones, or of elongated rods. In one embodiment, the particles may be irregular in shape. In one embodiment, a plurality of particles may consist essentially of spherical particles. Particles for use in the present disclosure may be PEGylated and/or aminated as otherwise described in PCT/US2014/56312 and PCT/US2014/56342, referenced above.

The term “cargo” is used herein to describe any molecule or compound, whether a small molecule or macromolecule having an activity relevant to its use in MSNPSs, protocells, and/or carriers, especially including biological activity, which can be included in MSNPs, protocells, and/or carriers according to the present disclosure. In principal embodiments of the present disclosure, the cargo is a nucleic acid sequence, such as ds plasmid DNA. In other embodiments, the cargo can express or encode siRNA, alone or in combination with other cargo as described herein. In preferred embodiments, the cargo is CRISPR ds plasmid DNA, which preferably expresses or encodes siRNA and/or one or more of mRNA, siRNA, shRNA, micro RNA, among other cargo. The siRNA is capable of producing apoptosis of a cancer cell. Examples of siRNA useful in the present application include S565, S7824, and/or s10234, among others. The cargo may be included within the pores and/or on the surface of the MSNP according to the present disclosure. Additional representative cargo may include, for example, a small molecule bioactive agent, a nucleic acid (e.g., RNA or DNA), a polypeptide, including a protein or a carbohydrate. Particular examples of such cargo include RNA, such as mRNA, siRNA, shRNA micro RNA, a polypeptide or protein, including a protein toxin (e.g., ricin toxin A-chain or diphtheria toxin A-chain) and/or DNA (including double stranded or linear DNA, complementary DNA (cDNA), minicircle DNA, naked DNA and plasmid DNA (especially CRISPR ds plasmid DNA which is modified to express RNA and/or a protein such as a reporter, e.g., green fluorescent protein, especially siRNA which causes apoptosis of cancer cells) which optionally may be supercoiled and/or packaged (e.g., with histones) and which may be optionally modified with a nuclear localization sequence). Cargo may also include a reporter as described herein.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2,000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. In certain aspects of the present disclosure, where administration via intravenous, intramuscular, intraperitoneal, retro-orbital and subcutaneous injection routes produces long residence times (on the order of at least 12 hours to 2 weeks or more) and greater biodistribution and/or bioavailability, the MSNPs, protocells, and/or carriers are monodisperse and generally no greater than about 50 nm in average diameter, often less than about 30 nm in average diameter, as otherwise described herein. The term “D₅₀” refers to the particle size below which 50% of the particles in a multiparticulate fall. Similarly, the term “D₉₀” refers to the particle size below which 90% of the particles in a multiparticulate fall.

The MSNP size distribution, according to the present disclosure, depends on the application, but is principally monodisperse (e.g., a uniform sized population varying no more than about 5-20% in diameter, as otherwise described herein). The term “monodisperse” is used as a standard definition established by the National Institute of Standards and Technology (NIST) (Particle Size Characterization, Special Publication 960-1, January 2001) to describe a distribution of particle size within a population of particles, in this case nanoparticles, which particle distribution may be considered monodisperse if at least 90% of the distribution lies within 5% of the median size. See Takeuchi S et al., Adv. Mater. 2005; 17(8):1067-72.

In certain embodiments, mesoporous silica nanoparticles can range, e.g., from around 1 nm to around 500 nm in size, including all integers and ranges there between. The size is measured as the longest axis of the particle. In various embodiments, the particles are from around 5 nm to around 500 nm and from around 10 nm to around 100 nm in size. The mesoporous silica nanoparticles have a porous structure. The pores can be from around 0.5 nm to about 25 nm in diameter, often about 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, preferred MSNPs according to the present disclosure: are monodisperse and range in size from about 25 nm to about 300 nm; exhibit stability (colloidal stability); have single cell binding specification to the substantial exclusion of non-targeted cells; are anionic, neutral or cationic for specific targeting (preferably cationic); are optionally modified with agents such as PEI, NMe³⁺, dye, crosslinker, ligands (ligands provide neutral charge); and optionally, are used in combination with a cargo to be delivered to a targeted cell.

In certain alternative embodiments, the MSNPs are monodisperse and range in size from about 25 nm to about 300 nm. The sizes used preferably include 50 nm (+/−10 nm) and 150 nm (+/−15 nm), within a narrow monodisperse range, but may be more narrow in range. A broad range of particles is not used because such a population is difficult to control and to target specifically.

In certain alternative embodiments, the present disclosure are directed to MSNPs and preferably, protocells, and/or carriers of a particular size (diameter) ranging from about 0.5 to about 30 nm, about 1 nm to about 30 nm, often about 5 nm to about 25 nm (preferably, less than about 25 nm), often about 10 to about 20 nm, for administration via intravenous, intramuscular, intraperitoneal, retro-orbital and subcutaneous injection routes. These MSNPs, protocells, and/or carriers are often monodisperse and provide colloidally stable compositions. These compositions can be used to target tissues in a patient or subject because of enhanced biodistribution/bioavailability of these compositions, and optionally, specific cells, with a wide variety of therapeutic and/or diagnostic agents which exhibit varying release rates at the site of activity.

The term “neoplasia” refers to the uncontrolled and progressive multiplication of tumor cells, under conditions that would not elicit, or would cause cessation of, multiplication of normal cells. Neoplasia results in a “neoplasm”, which is defined herein to mean any new and abnormal growth, particularly a new growth of tissue, in which the growth of cells is uncontrolled and progressive. Thus, neoplasia includes “cancer”, which herein refers to a proliferation of tumor cells having the unique trait of loss of normal controls, resulting in unregulated growth, lack of differentiation, local tissue invasion, and/or metastasis.

As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of anaplasia, or loss of differentiation and orientation of cells, and have the properties of invasion and metastasis. Examples of neoplasms or neoplasias from which the target cell of the present disclosure may be derived include, without limitation, carcinomas (e.g., squamous-cell carcinomas, adenocarcinomas, hepatocellular carcinomas, and renal cell carcinomas), particularly those of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, and melanoma); mixed types of neoplasias, particularly carcinosarcoma and Hodgkin's disease; and tumors of mixed origin, such as Wilms' tumor and teratocarcinomas (Beers and Berkow (eds.), The Merck Manual of Diagnosis and Therapy, 17^{th} ed. (Whitehouse Station, N.J.: Merck Research Laboratories, 1999) 973-74, 976, 986, 988, 991.

The term “anticancer agent” or “additional anticancer agent” (depending on the context of its use) shall mean chemotherapeutic agents such as an agent selected from the group consisting of microtubule-stabilizing agents, microtubule-disruptor agents, alkylating agents, antimetabolites, epidophyllotoxins, antineoplastic enzymes, topoisomerase inhibitors, inhibitors of cell cycle progression, and platinum coordination complexes. These may be selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib, PD0325901, AZD-6244, capecitabine, L-glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258, 3-[5-(methylsulfonylpiperadinemethyl)-indolyl]-quinolone, vatalanib, AG-013736, AVE-0005, the acetate salt of [D-Ser(But)6,Azgly 10] (pyro-Glu-His-Trp-Ser-Tyr-D-Ser(But)-Leu-Arg-Pro-Azgly-NH₂ acetate [C₅₉H₈₄N₁₈O₁₄—(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714, TAK-165, HKI-272, erlotinib, lapatanib, canertinib, ABX-EGF antibody, erbitux, EKB-569, PKI-166, GW-572016, lonafarnib, BMS-214662, tipifarnib, amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, vairubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox,gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-1, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, and darbepoetin alfa, among others.

MSNPs, protocells, and/or carriers of the disclosure also can comprise anticancer agents selected from the group consisting of doxorubicin-loaded liposomes that are functionalized by polyethylene glycol (PEG), antimetabolites, inhibitors of topoisomerase I and II, alkylating agents and microtubule inhibitors, Adriamycin; aldesleukin; alemtuzumab; alitretinoin; allopurinol; altretamine; amifostine; anastrozole; arsenic trioxide; Asparaginase; BCG Live; bexarotene capsules; bexarotene gel; bleomycin; busulfan intravenous; busulfan oral; calusterone; capecitabine; carboplatin; carmustine; carmustine with Polifeprosan 20 Implant; celecoxib; chlorambucil; cisplatin; cladribine; cyclophosphamide; cytarabine; cytarabine liposomal; dacarbazine; dactinomycin; actinomycin D; Darbepoetin alfa; daunorubicin liposomal; daunorubicin, daunomycin; Denileukin diftitox, dexrazoxane; docetaxel; doxorubicin; doxorubicin liposomal; Dromostanolone propionate; Elliott's B Solution; epirubicin; Epoetin alfa estramustine; etoposide phosphate; etoposide (VP-16); exemestane; Filgrastim; floxuridine (intraarterial); fludarabine; fluorouracil (5-FU); fulvestrant; gemcitabine, gemtuzumab ozogamicin; goserelin acetate; hydroxyurea; Ibritumomab Tiuxetan; idarubicin; ifosfamide; imatinib mesylate; Interferon alfa-2a; Interferon alfa-2b; irinotecan; letrozole; leucovorin; levamisole; lomustine (CCNU); meclorethamine (nitrogen mustard); megestrol acetate; melphalan (L-PAM); mercaptopurine (6-MP); mesna; methotrexate; methoxsalen; mitomycin C; mitotane; mitoxantrone; nandrolone phenpropionate; Nofetumomab; LOddC; Oprelvekin; oxaliplatin; paclitaxel; pamidronate; pegademase; Pegaspargase; Pegfilgrastim; pentostatin; pipobroman; plicamycin; mithramycin; porfimer sodium; procarbazine; quinacrine; Rasburicase; Rituximab; Sargramostim; streptozocin; talbuvidine (LDT); talc; tamoxifen; temozolomide; teniposide (VM-26); testolactone; thioguanine (6-TG); thiotepa; topotecan; toremifene; Tositumomab; Trastuzumab; tretinoin (ATRA); uracil mustard; vairubicin; valtorcitabine (monoval LDC); vinblastine; vinorelbine; zoledronate; and mixtures thereof.

In certain embodiments, MSNPs, protocells, and/or carriers of the disclosure, in addition to having ds DNA (especially CRISPR plasmid DNA expressing siRNA and other RNA as cargo), also comprise anticancer drugs including anticancer drugs selected from the group consisting of doxorubicin, melphalan, bevacizumab, dactinomycin, cyclophosphamide, doxorubicin liposomal, amifostine, etoposide, gemcitabine, altretamine, topotecan, cyclophosphamide, paclitaxel, carboplatin, cisplatin, and taxol.

MSNPs, protocells, and/or carriers of the disclosure can include one or more antiviral agents to treat viral infections, especially including HIV infections, HBV infections and/or HCV infections. Exemplary anti-HIV agents include, for example, nucleoside reverse transcriptase inhibitors (NRTI), non-nucleoside reverse transcriptase inhibitors (NNRTI), protease inhibitors, fusion inhibitors, among others, exemplary compounds of which may include, for example, 3TC (Lamivudine), AZT (Zidovudine), (−)-FTC, ddI (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof, including anti-HIV compounds presently in clinical trials or in development. Exemplary anti-HBV agents include, for example, hepsera (adefovir lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Anti-HCV agents include, for example, interferon, pegylated intergeron, ribavirin, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NSSA, NS4B, ANA598, A-689, GNI-104, IDX102, ADX184, GL59728, GL60667, PSI-7851, TLR9 Agonist, PHX1766, SP-30 and mixtures thereof.

Other exemplary antiviral agents include broad spectrum antiviral agents, antibodies, small molecule antiviral agents, antiretroviral agents, etc. Further non-limiting antiviral agents include abacavir, ACH-3102, acyclovir (acyclovir), acyclovir, adefovir, amantadine, amprenavir, ampligen, arbidol, asunaprevir, atazanavir, atripla, balavir, BCX4430, boceprevir, brincidofovir, brivudine, cidofovir, clevudine, combivir, cytarabine, daclatasvir, dasabuvir, deleobuvir, dolutegravir, darunavir, delavirdine, didanosine, docosanol, edoxudine, efavirenz, elbasvir, emtricitabine, enfuvirtide, entecavir, ecoliever, faldaprevir, famciclovir, favipiravir, fomivirsen, fosamprenavir, foscarnet, fosfonet, ganciclovir, grazoprevir, ibacitabine, imunovir, idoxuridine, imiquimod, indinavir, interferon type III, interferon type II, interferon type I, interferon, interferon alfa 2b, lamivudine, laninamivir, ledipasvir (with or without sofosbuvir), lopinavir, loviride, maraviroc, moroxydine, methisazone, MK-3682, MK-8408, nelfinavir, nevirapine, nexavir, novir, ombitasvir (with or without paritaprevir and/or ritonavir), oseltamivir (Tamiflu), paritaprevir, peginterferon alfa-2a, penciclovir, peramivir, pleconaril, podophyllotoxin, raltegravir, resiquimod, ribavirin, rifampicin, rimantadine, ritonavir, pyramidine, samatasvir, saquinavir, simeprevir, sofosbuvir, stavudine, taribavirin, tecovirimat (ST-246), telaprevir, telbivudine, tenofovir, tenofovir disoproxil, tipiracil, tipranavir, trifluridine (with or without tipiracil), trizivir, tromantadine, truvada, umifenovir, valaciclovir (Valtrex), valganciclovir, vicriviroc, vidarabine, viramidine, zalcitabine, zanamivir (Relenza), zidovudine, including prodrugs, salts, and/or combinations thereof.

The above compounds/bioactive agents may also be charged to MSNPs, preferably including protocells, and/or carriers having average diameters which are less than about 50 nm, more preferably less than 30 nm for formulating compositions adapted for intravenous, intramuscular, intraperitoneal, retro-orbital and subcutaneous injection routes. In certain embodiments, subcutaneous routes of administration are preferred for administering bioactive agents.

The terms “targeting ligand” and “targeting active species” are used to describe a compound or moiety (preferably an antigen), which is complexed or preferably covalently bonded to the surface of MSNPs, protocells, and/or carriers according to the present disclosure which binds to a moiety on the surface of a cell to be targeted so that the MSNPs, protocells, and/or carriers may selectively bind to the surface of the targeted cell and deposit their contents into the cell. The targeting active species for use in the present disclosure is preferably a targeting peptide as otherwise described herein, a polypeptide including an antibody or antibody fragment, an aptamer, or a carbohydrate, among other species which bind to a targeted cell.

Preferred ligands which may be used to target cells include peptides, affibodies, and antibodies (including monoclonal and/or polyclonal antibodies). In certain embodiments, targeting ligands selected from the group consisting of Fcγ from human IgG (which binds to Fcγ receptors on macrophages and dendritic cells), human complement C3 (which binds to CR1 on macrophages and dendritic cells), ephrin B2 (which binds to EphB4 receptors on alveolar type II epithelial cells), and the SP94 peptide (which binds to unknown receptor(s) on hepatocyte-derived cells). Exemplary, non-limiting SP94 peptides include SP94 free peptide (H₂N-SFSIILTPILPL-COOH, SEQ ID NO:126), a SP94 peptide modified with C-terminal Cys for conjugation (H₂N-SFSIILTPILPLGGC-COOH, SEQ ID NO:127), and a further modified SP94 peptide (H2N-SFSIILTPILPLEEEGGC-COOH, SEQ ID NO:128)

The term “MET binding peptide” or “MET receptor binding peptide” includes, but is not limited to, five (5) 7-mer peptides which have been shown to bind MET receptors on the surface of cancer cells with enhanced binding efficiency. Pursuant to the present disclosure, several small peptides with varying amino acid sequences were identified which bind the MET receptor (a.k.a. hepatocyte growth factor receptor, expressed by gene c-MET) with varying levels of specificity and with varying ability to activate MET receptor signaling pathways. 7-mer peptides were identified using phage display biopanning, with examples of resulting sequences which evidence enhanced binding to MET receptor and consequently to cells such as cancer cells (e.g., hepatocellular, ovarian and cervical) which express high levels of MET receptors, which appear below. Binding data for several of the most commonly observed sequences during the biopanning process is also presented in the examples section of the present application. These peptides are particularly useful as targeting ligands for cell-specific therapeutics. However, peptides with the ability to activate the receptor pathway may have additional therapeutic value themselves or in combination with other therapies. Many of the peptides have been found bind not only hepatocellular carcinoma, which was the original intended target, but also to bind a wide variety of other carcinomas including ovarian and cervical cancer. These peptides are believed to have wide-ranging applicability for targeting or treating a variety of cancers and other physiological problems associated with expression of MET and associated receptors.

The following five 7mer peptide sequences show substantial binding to MET receptor and are particularly useful as targeting peptides for use on protocells or carriers according to the present disclosure.

SEQ ID NO: 121
ASVHFPP (Ala-Ser-Val-His-Phe-Pro-Pro)
SEQ ID NO: 122
TATFWFQ (Thr-Ala-Thr-Phe-Trp-Phe-Gln)
SEQ ID NO: 123
TSPVALL (Thr-Ser-Pro-Val-Ala-Leu-Leu)
SEQ ID NO: 124
IPLKVHP (Ile-Pro-Leu-Lys-Val-His-Pro)
SEQ ID NO: 125
WPRLTNM (Trp-Pro-Arg-Leu-Thr-Asn-Met)

Each of these peptides may be used alone or in combination with other MET peptides within the above group or with other targeting peptides which may assist in binding protocells or carriers according to the present disclosure to cancer cells, including hepatocellular cancer cells, ovarian cancer cells and cervical cancer cells, among numerous others. These binding peptides may also be used in pharmaceutical compounds alone as MET binding peptides to treat cancer and otherwise inhibit hepatocyte growth factor binding.

The terms “cell penetration peptide”, “fusogenic peptide”, and “endosomolytic peptide” are used to describe a peptide, which aids MSNP or protocell or carrier translocation across a lipid bilayer, such as a cellular membrane or endosome lipid bilayer and in the present disclosure is optionally crosslinked onto a lipid bilayer surface of the protocells or carriers according to the present disclosure. Endosomolytic peptides are a sub-species of fusogenic peptides as described herein. In both the multilamellar and single layer protocell or carrier embodiments, the non-endosomolytic fusogenic peptides (e.g., electrostatic cell penetrating peptide such as R8 octaarginine) are incorporated onto the protocells or carriers at the surface of the protocell or carrier in order to facilitate the introduction of protocells or carriers into targeted cells (APCs) to effect an intended result (to instill an immunogenic and/or therapeutic response as described herein). The endosomolytic peptides (often referred to in the art as a subset of fusogenic peptides) may be incorporated in the surface lipid bilayer of the protocell or carrier or in a lipid sublayer of the multilamellar protocell or carrier in order to facilitate or assist in the escape of the protocell or carrier from endosomal bodies. Representative and preferred electrostatic cell penetration (fusogenic) peptides for use in protocells or carriers according to the present disclosure include an 8 mer polyarginine (NH₂-RRRRRRRR-COOH, SEQ ID NO:1), among others known in the art, which are included in protocells according to the present disclosure in order to enhance the penetration of the protocell or carrier into cells. Representative endosomolytic fusogenic peptides (“endosomolytic peptides”) include H5WYG peptide (NH2-GLFHAIAHFIHGGWHGLIHGWYGGC-COOH, SEQ ID NO:2), RALA peptide (NH2-WEARLARALARALARHLARALARALRAGEA-COOH, SEQ ID NO:3), KALA peptide (NH₂-WEAKLAKALAKALAKHLAKALAKALKAGEA-COOH), SEQ ID NO:4), GALA (NH₂-WEAALAEALAEALAEHLAEALAEALEALAA-COOH, SEQ ID NO:5) and INF7 (NH2-GLFEAIEGFIENGWEGMIDGWYG-COOH, SEQ ID NO:6), or fragments thereof, among others.

The charge is controlled based on what is to be accomplished (via PEI, NMe³⁺, dye, crosslinker, ligands, etc.), but for targeting the charge is preferably cationic. Charge also changes throughout the process of formation. Initially the targeted particles are cationic and are often delivered as cationically charged nanoparticles, however post modification with ligands they are closer to neutral. The ligands which find use in the present disclosure include peptides, affibodies, and antibodies, among others. These ligands are site specific and are useful for targeting specific cells which express peptides to which the ligand may bind selectively to targeted cells.

MSNPs pursuant to the present disclosure may be used to deliver cargo to a targeted cell, including, for example, cargo component selected from the group consisting of at least one polynucleotide, such as double stranded linear DNA, minicircle DNA, naked DNA or plasmid DNA (especially CRISPR ds plasmid DNA, RNA, as well as chimeras, fusions, or modified forms thereof), messenger RNA, small interfering RNA, small hairpin RNA, microRNA, a polypeptide (e.g., a recruitment domain or fragments thereof), a protein (e.g., an enzyme, an initiation factor, or fragments thereof), a drug (in particular, an anticancer drug such as a chemotherapeutic agent), an imaging agent, a detection agent (e.g., a dye, such as an electroactive detection agent, a fluorescent dye, a luminescent dye, a chemiluminescent dye, a colorimetric dye, a radioactive agent, etc.), a label (e.g., a fluorescent label, a colorimetric label, a quantum dot, a nanoparticle, a microparticle, an electroactive label, an electrocatalytic label, a barcode, a radio label (e.g., an RF label or barcode), avidin, biotin, a tag, a dye, a marker, an enzyme or protein that can optionally include one or more linking agents and/or one or more dyes), or a mixture thereof. The MSNPs pursuant to the present disclosure are effective for accommodating cargo which are long and thin (e.g., naked) in three-dimensional structure, such as polynucleotides (e.g., various DNA and RNA) and polypeptides.

Protocells and carriers of the disclosure are highly flexible and modular. High concentrations of physiochemically-disparate molecules can be loaded into the protocells or carriers and their therapeutic and/or diagnostic agent release rates can be optimized without altering the protocell's or carrier's size, size distribution, stability, or synthesis strategy. Properties of the supported lipid bi- or multilayer and mesoporous silica nanoparticle core can also be modulated independently, thereby optimizing properties as surface charge, colloidal stability, and targeting specificity independently from overall size, type of cargo(s), loading capacity, and release rate.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

Treatment, as used herein, encompasses both prophylactic and therapeutic treatment, principally of cancer, but also of other disease states, including bacterial and viral infections, (e.g., HBV and/or HCV). Compounds according to the present disclosure can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present disclosure can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present disclosure is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections.

MSNPs, protocells, and/or carriers can also be used to treat a wide variety of bacterial infections including, but not limited to, infections caused by bacteria selected from the group consisting of F. tularensis, B. pseudomallei, Mycobacterium, staphylococcus, streptococcaceae, neisseriaaceae, cocci, enterobacteriaceae, pseudomonadaceae, vibrionaceae, campylobacter, pasteurellaceae, bordetella, francisella, brucella, legionellaceae, bacteroidaceae, gram-negative bacilli, clostridium, corynebacterium, propionibacterium, gram-positive bacilli, anthrax, actinomyces, nocardia, mycobacterium, treponema, borrelia, leptospira, mycoplasma, ureaplasma, rickettsia, chlamydiae, and P. aeruginosa.

Antibiotic MSNPs, protocells, and/or carriers of the disclosure can contain one or more antibiotics or antibacterial agents, e.g., “Antibiotics” include, but are not limited to, compositions selected from the group consisting of Gentamicin, Kanamycin, Neomycin, Netilmicin, Tobramycin, Paromomycin, Spectinomycin, Geldanamycin, Herbimycin, Rifaximin, Streptomycin, Ertapenem, Doripenem, Imipenem/Cilastatin, Meropenem, Cefadroxil, Cefazolin, Cephalothin, Cephalexin,Cefaclor, Cefamandole, Cefoxitin, Cefprozil, Cefuroxime, Cefixime, Cefdinir, Cefditoren, Cefoperazone Cefotaxime, Cefpodoxime, Ceftazadime, Ceftibuten, Ceftizoxime Ceftriaxone, Cefepime, Ceftaroline fosamil, Ceftobiprole, Teicoplanin, Vancomycin, Telavancin, Daptomycin, Oritavancin, WAP-8294A, Azithromycin, Clarithromycin, Dirithromycin, Erythromycin, Roxithromycin, Telithromycin, Spiramycin, Clindamycin, Lincomycin, Aztreonam, Furazolidone, Nitrofurantoin, Oxazolidonones, Linezolid, Posizolid, Radezolid, Torezolid, Amoxicillin, Ampicillin, Azlocillin, Carbenicillin, Cloxacillin Dicloxacillin, Flucloxacillin, Mezlocillin, Methicillin, Nafcillin, Oxacillin, Penicillin G, Penicillin V, Piperacillin, Temocillin, Ticarcillin, Amoxicillin/clavulanate, Ampicillin/sulbactam, Piperacillin/tazobactam, Ticarcillin/clavulanate, Bacitracin, Colistin, Polymyxin B, Ciprofloxacin, Enoxacin, Gatifloxacin, Gemifloxacin, Levofloxacin, Lomefloxacin, Moxifloxacin, Nalidixic acid, Norfloxacin, Ofloxacin, Trovafloxacin, Grepafloxacin, Sparfloxacin, Mafenide, Sulfacetamide, Sulfadiazine, Sulfadimethoxine, Sulfamethizole, Sulfamethoxazole, Sulfasalazine, Sulfisoxazole, Trimethoprim-Sulfamethoxazole, Sulfonamidochrysoidine, Demeclocycline, Doxycycline, Vibramycin Minocycline, Tigecycline, Oxytetracycline, Tetracycline, Clofazimine, Capreomycin, Cycloserine, Ethambutol, Rifampicin, Rifabutin, Rifapentine, Arsphenamine, Chloramphenicol, Fosfomycin, Fusidic acid, Metronidazole, Mupirocin, Platensimycin, Quinupristin/Dalfopristin, Thiamphenicol, Tigecycline and Tinidazole and combinations thereof.

The term “lipid” is used to describe the components which are used to form lipid bi- or multilayers on the surface of the particles which are used in the present disclosure (e.g., as protocells or as carriers) and may include a PEGylated lipid. Various embodiments provide nanostructures which are constructed from nanoparticles which support a lipid bilayer(s). In embodiments according to the present disclosure, the nanostructures preferably include, for example, a core-shell structure including a porous particle core surrounded by a shell of lipid bilayer(s). The nanostructure, preferably a porous alum nanostructure as described above, supports the lipid bilayer membrane structure.

The lipid bi- or multilayer supported on the porous particle according to one embodiment of the present disclosure has a lower melting transition temperature, i.e., is more fluid than a lipid bi- or multilayer supported on a non-porous support or the lipid bi- or multilayer in a liposome. This is sometimes important in achieving high affinity binding of immunogenic peptides or targeting ligands at low peptide densities, as it is the bilayer fluidity that allows lateral diffusion and recruitment of peptides by target cell surface receptors. One embodiment provides for peptides to cluster, which facilitates binding to a complementary target.

In the present disclosure, the lipid bi- or multilayer may vary significantly in composition. Ordinarily, any lipid or polymer which may be used in liposomes may also be used in MSNPs according to the present disclosure. Preferred lipids are as otherwise described herein.

In embodiments according to the disclosure, the lipid bi- or multilayer of the protocells or the carriers can provide biocompatibility and can be modified to possess targeting species including, for example, antigens, targeting peptides, fusogenic peptides, antibodies, aptamers, and PEG (polyethylene glycol) to allow, for example, further stability of the protocells or carriers and/or a targeted delivery into a cell to maximize an immunogenic response. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used) and the PEG component which is generally conjugated to phospholipid through an amine group comprises about 1% to about 20%, preferably about 5% to about 15%, about 10% by weight of the lipids which are included in the lipid bi- or multilayer. The PEG component is generally conjugated to an amine-containing lipid such as DOPE or DPPE or other lipid, but in alternative embodiments may also be incorporated into the MSNPs, through inclusion of a PEG containing silane.

Numerous lipids which are used in liposome delivery systems may be used to form the lipid bi- or multilayer on particles (e.g., nanoparticles) to provide MSNPS, protocells, and/or carriers according to the present disclosure. Virtually any lipid which is used to form a liposome may be used in the lipid bi- or multilayer which surrounds the particles to form MSNPS, protocells, and/or carriers according to an embodiment of the present disclosure. Preferred lipids for use in the present disclosure include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-lphosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-[12-[(7-nitro-2-1,3-benzoxadiazol-4-yl)amino]lauroyl]-sn-glycero-3-phosphocholine (16:0-12:0 NBD PC), cholesterol and mixtures/combinations thereof. Cholesterol, not technically a lipid, but presented as a lipid for purposes of an embodiment of the present disclosure given the fact that cholesterol may be an important component of the lipid bilayer of protocells or carriers according to an embodiment of the disclosure. Often cholesterol is incorporated into lipid bilayers of protocells or carriers in order to enhance structural integrity of the bilayer. These lipids are all readily available commercially from Avanti Polar Lipids, Inc. (Alabaster, Alabama, USA). DOPE and DPPE are particularly useful for conjugating (through an appropriate crosslinker) PEG, peptides, polypeptides, including immunogenic peptides, proteins and antibodies, RNA and DNA through the amine group on the lipid.

MSNPs, protocells, and/or carriers of the disclosure can be PEGylated with a variety of polyethylene glycol-containing compositions as described herein. PEG molecules can have a variety of lengths and molecular weights and include, but are not limited to, PEG 200, PEG 1000, PEG 1500, PEG 4600, PEG 10,000, PEG-peptide conjugates or combinations thereof.

The term “reporter” is used to describe an imaging agent or moiety which is incorporated into the phospholipid bilayer or cargo of MSNPs according to an embodiment of the present disclosure and provides a signal which can be measured. The moiety may provide a fluorescent signal or may be a radioisotope which allows radiation detection, among others. Exemplary fluorescent labels for use in MSNPs, protocells, and/or carriers (preferably via conjugation or adsorption to the lipid bi- or multilayer or silica core, although these labels may also be incorporated into cargo elements such as DNA, RNA, polypeptides and small molecules which are delivered to cells by the protocells or carriers) include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580), Alexa Fluor® 532 carboxylic acid, succinimidyl ester(532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art.

Additional reporters include polypeptide reporters which may be expressed by plasmids (such as histone-packaged supercoiled DNA plasmids) and include polypeptide reporters such as fluorescent green protein and fluorescent red protein. Reporters pursuant to the present disclosure are utilized principally in diagnostic applications including diagnosing the existence or progression of cancer (cancer tissue) in a patient and or the progress of therapy in a patient or subject.

Pharmaceutical compositions according to the present disclosure comprise an effective population of MSNPs, protocells, and/or carriers as otherwise described herein formulated to effect an intended result (e g , immunogenic result, therapeutic result and/or diagnostic analysis, including the monitoring of therapy) formulated in combination with a pharmaceutically acceptable carrier, additive or excipient. The MSNPs, protocells, and/or carriers within the population of the composition may be the same or different depending upon the desired result to be obtained. Pharmaceutical compositions according to the present disclosure may also comprise an addition bioactive agent or drug, such as an antibiotic or antiviral agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g., orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the disclosure include humans, companion animals, laboratory animals, and the like. The disclosure contemplates immediate and/or sustained/controlled release compositions, including compositions which comprise both immediate and sustained release formulations. This is particularly true when different populations of MSNPs, protocells, and/or carriers are used in the pharmaceutical compositions or when additional bioactive agent(s) are used in combination with one or more populations of protocells or carriers as otherwise described herein.

Formulations containing the compounds according to the present disclosure may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present disclosure typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the disclosure, with the remainder consisting essentially of suitable pharmaceutical excipients.

An injectable composition for parenteral administration (e.g., intravenous, intramuscular, or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of MSNPs, protocells, and/or carriers (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g., an excipient such as a saccharide or cellulose preparation, a binder such as starch paste or methyl cellulose, a filler, a disintegrator, and other additives typically used in the manufacture of medical preparations.

Methods for preparing such dosage forms are known or apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present disclosure.

Methods of treating patients or subjects in need for a particular disease state or infection comprise administration an effective amount of a pharmaceutical composition comprising therapeutic MSNPs, protocells, and/or carriers and optionally at least one additional bioactive (e.g., antiviral) agent according to the present disclosure.

Diagnostic methods according to the present disclosure comprise administering to a patient in need an effective amount of a population of diagnostic MSNPs, protocells, and/or carriers (e.g., MSNPs, protocells, and/or carriers which comprise a target species, such as a targeting peptide which binds selectively to cancer cells and a reporter component to indicate the binding of the protocells or carriers) whereupon the binding of the MSNPs, protocells, and/or carriers to cells as evidenced by the reporter component (moiety) will enable a diagnosis of the existence of a disease state in the patient.

An alternative of the diagnostic method of the present disclosure can be used to monitor the therapy of a disease state in a patient, the method comprising administering an effective population of diagnostic MSNPs, protocells, and/or carriers (e.g., MSNPs, protocells, and/or carriers which comprise a target species, such as a targeting peptide which binds selectively to target cells and a reporter component to indicate the binding of the protocells or carriers to cancer cells if the cancer cells are present) to a patient or subject prior to treatment, determining the level of binding of diagnostic protocells or carriers to target cells in said patient and during and/or after therapy, determining the level of binding of diagnostic protocells or carriers to target cells in said patient, whereupon the difference in binding before the start of therapy in the patient and during and/or after therapy will evidence the effectiveness of therapy in the patient, including whether the patient has completed therapy or whether the disease state has been inhibited or eliminated.

In accordance with the present disclosure there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL.

By “about” is meant ±10% of the recited value. Further, reference to “about” a value or parameter herein includes (and describes) variations that are directed to that value or parameter per se. For example, description referring to “about X” includes description of “X”.

By “micro” is meant having at least one dimension that is less than 1 mm. For instance, a microstructure (e.g., any structure described herein) can have a length, width, height, cross-sectional dimension, circumference, radius (e.g., external or internal radius), or diameter that is less than 1 mm.

By “nano” is meant having at least one dimension that is less than 1 μm. For instance, a nanostructure (e.g., any structure described herein) can have a length, width, height, cross-sectional dimension, circumference, radius (e.g., external or internal radius), or diameter that is less than 1 μm.

The terms “polynucleotide” and “nucleic acid,” used interchangeably herein, refer to a polymeric form of nucleotides of any length, either ribonucleotides or deoxyribonucleotides. Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, DNA-RNA hybrids, or a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs.

The term “modified,” as used in reference to nucleic acids, means a nucleic acid sequence including one or more modifications to the nucleobase, nucleoside, nucleotide, phosphate group, sugar group, and/or internucleoside linkage (e.g., phosphodiester backbone, linking phosphate, or a phosphodiester linkage).

The nucleoside modification may include, but is not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine, and combinations thereof.

A sugar modification may include, but is not limited to, a locked nucleic acid (LNA, in which the 2′-hydroxyl is connected by a C₁₋₆ alkylene or C₁₋₆ heteroalkylene bridge to the 4′-carbon of the same ribose sugar), replacement of the oxygen in ribose (e.g., with S, Se, or alkylene, such as methylene or ethylene), addition of a double bond (e.g., to replace ribose with cyclopentenyl or cyclohexenyl), ring contraction of ribose (e.g., to form a 4-membered ring of cyclobutane or oxetane), ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone), multicyclic forms (e.g., tricyclic), and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with a-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar.

A backbone modification may include, but is not limited to, 2′-deoxy- or 2′-O-methyl modifications. A phosphate group modification may include, but is not limited to, phosphorothioate, phosphoroselenates, boranophosphates, boranophosphate esters, hydrogen phosphonates, phosphoramidates, phosphorodiamidates, alkyl or aryl phosphonates, phosphotriesters, phosphorodithioates, bridged phosphoramidates, bridged phosphorothioates, or bridged methylene-phosphonates.

“Complementarity” or “complementary” refers to the ability of a nucleic acid to form hydrogen bond(s) with another nucleic acid sequence by either traditional Watson-Crick or other non-traditional types, e.g., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” or “sufficient complementarity” as used herein refers to a degree of complementarity that is at least 60%, 65%, 70%, 75%, 80%, 85%, 90%, 95%. 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part 1, Second Chapter “Overview of principles of hybridization and the strategy of nucleic acid probe assay”, Elsevier, N.Y.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. Hybridization and washing conditions are well known and exemplified in Sambrook J, Fritsch E F, and Maniatis T, “Molecular Cloning: A Laboratory Manual,” Second Edition, Cold Spring Harbor Laboratory Press, Cold Spring Harbor (1989), particularly Chapter 11 and Table 11.1 therein; and Sambrook J and Russell W, “Molecular Cloning: A Laboratory Manual,” Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g., complementarity over 35 or less, 30 or less, 25 or less, 22 or less, 20 or less, or 18 or less nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid are: at least about 15 nucleotides; at least about 20 nucleotides; at least about 22 nucleotides; at least about 25 nucleotides; and at least about 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise at least 70%, at least 80%, at least 90%, at least 95%, at least 99%, or 100% sequence complementarity to a target region within the target nucleic acid sequence to which they are targeted. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST programs (basic local alignment search tools) and PowerBLAST programs known in the art (Altschul SF et al., J. Mol. Biol. 1990; 215:403-10; Zhang J et al., Genome Res. 1997; 7:649-56) or by using the Gap program (Wisconsin Sequence Analysis Package, Version 8 for Unix, Genetics Computer Group, University Research Park, Madison Wis.), using default settings, which uses the algorithm of Smith TF et al., Adv. Appl. Math. 1981; 2(4):482-9).

By “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones.

The term “fragment” is meant a portion of a nucleic acid or a polypeptide that is at least one nucleotide or one amino acid shorter than the reference sequence. This portion contains, preferably, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides; or 10, 20, 30, 40, 50, 60, 70, 80, 90, 100, 150, 200, 250, 300, 350, 400, 450, 500, 550, 600, 640 amino acids or more. In another example, any polypeptide fragment can include a stretch of at least about 5 (e.g., about 10, about 20, about 30, about 40, about 50, or about 100) amino acids that are at least about 40% (e.g., about 50%, about 60%, about 70%, about 80%, about 90%, about 95%, about 87%, about 98%, about 99%, or about 100%) identical to any of the sequences described herein can be utilized in accordance with the disclosure. In certain embodiments, a polypeptide to be utilized in accordance with the disclosure includes 2, 3, 4, 5, 6, 7, 8, 9, 10, or more mutations (e.g., one or more conservative amino acid substitutions, as described herein). In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the disclosure.

The term “conservative amino acid substitution” refers to the interchangeability in proteins of amino acid residues having similar side chains (e.g., of similar size, charge, and/or polarity). For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamic acid and aspartic acid; and a group of amino acids having sulfur containing side chains consists of cysteine and methionine. Exemplary conservative amino acid substitution groups are valine-leucine-isoleucine, phenylalanine-tyrosine, lysine-arginine, alanine-valine, glycine-serine, glutamate-aspartate, and asparagine-glutamine.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2(4):482-9) as incorporated into GeneMatcher Plus™ (Schwarz and Dayhof, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to a uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis., 53705). Such software may match similar sequences by assigning degrees of homology to various substitutions, deletions, and other modifications.

The term “chimeric” as used herein as applied to a nucleic acid or polypeptide refers to two components that are defined by structures derived from different sources. For example, where “chimeric” is used in the context of a chimeric polypeptide (e.g., a chimeric Cas9/Csn1 protein), the chimeric polypeptide includes amino acid sequences that are derived from different polypeptides. A chimeric polypeptide may comprise either modified or naturally-occurring polypeptide sequences (e.g., a first amino acid sequence from a modified or unmodified Cas9/Csn1 protein; and a second amino acid sequence other than the Cas9/Csn1 protein). Similarly, “chimeric” in the context of a polynucleotide encoding a chimeric polypeptide includes nucleotide sequences derived from different coding regions (e.g., a first nucleotide sequence encoding a modified or unmodified Cas9/Csn1 protein; and a second nucleotide sequence encoding a polypeptide other than a Cas9/Csn1 protein).

The term “chimeric polypeptide” refers to a polypeptide which is made by the combination (i.e., “fusion”) of two otherwise separated segments of amino sequence, usually through human intervention. A polypeptide that comprises a chimeric amino acid sequence is a chimeric polypeptide. Some chimeric polypeptides can be referred to as “fusion variants.”

“Heterologous,” as used herein, means a nucleotide or polypeptide sequence that is not found in the native nucleic acid or protein, respectively. For example, in a chimeric Cas9/Csn1 protein, the RNA-binding domain of a naturally-occurring bacterial Cas9/Csn1 polypeptide (or a variant thereof) may be fused to a heterologous polypeptide sequence (i.e., a polypeptide sequence from a protein other than Cas9/Csn1 or a polypeptide sequence from another organism). The heterologous polypeptide sequence may exhibit an activity (e.g., enzymatic activity) that will also be exhibited by the chimeric Cas9/Csn1 protein (e.g., methyltransferase activity, acetyltransferase activity, kinase activity, ubiquitinating activity, etc.). A heterologous nucleic acid sequence may be linked to a naturally-occurring nucleic acid sequence (or a variant thereof) (e.g., by genetic engineering) to generate a chimeric nucleotide sequence encoding a chimeric polypeptide. As another example, in a fusion variant Cas9 site-directed polypeptide, a variant Cas9 site-directed polypeptide may be fused to a heterologous polypeptide (i.e., a polypeptide other than Cas9), which exhibits an activity that will also be exhibited by the fusion variant Cas9 site-directed polypeptide. A heterologous nucleic acid sequence may be linked to a variant Cas9 site-directed polypeptide (e.g., by genetic engineering) to generate a nucleotide sequence encoding a fusion variant Cas9 site-directed polypeptide.

“Recombinant,” as used herein, means that a particular nucleic acid, as defined herein, is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., DNA-targeting RNA) that is not translated may also be considered recombinant Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “target sequence” as used herein is a polynucleotide (e.g., as defined herein, including a DNA, RNA, or DNA/RNA hybrid, as well as modified forms thereof) that includes a “target site.” The terms “target site” or “target protospacer DNA” are used interchangeably herein to refer to a nucleic acid sequence present in a target genomic sequence (e.g., DNA or RNA in a host or pathogen) to which a targeting portion of the guiding component will bind provided sufficient conditions (e.g., sufficient complementarity) for binding exist. Suitable DNA/RNA binding conditions include physiological conditions normally present in a cell. Other suitable DNA/RNA binding conditions (e.g., conditions in a cell-free system) are known in the art; see, e.g., Sambrook, supra.

By “cleavage” it is meant the breakage of the covalent backbone of a target sequence (e.g., a nucleic acid molecule). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, a complex comprising a guiding component and a nuclease is used for targeted double-stranded DNA cleavage. In other embodiments, a complex comprising a guiding component and a nuclease is used for targeted single-stranded RNA cleavage.

“Nuclease” and “endonuclease” are used interchangeably herein to mean an enzyme which possesses catalytic activity for DNA cleavage and/or RNA cleavage.

By “cleavage domain” or “active domain” or “nuclease domain” of a nuclease it is meant the polypeptide sequence or domain within the nuclease which possesses the catalytic activity for nucleic acid cleavage. A cleavage domain can be contained in a single polypeptide chain or cleavage activity can result from the association of two (or more) polypeptides. A single nuclease domain may consist of more than one isolated stretch of amino acids within a given polypeptide.

In some embodiments, the guiding component comprises a modification or sequence that provides for an additional desirable feature (e.g., modified or regulated stability; subcellular targeting; tracking, e.g., a fluorescent label; a binding site for a protein or protein complex; etc.). Non-limiting examples include: a short motif (referred to as the protospacer adjacent motif (PAM)); a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin)); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, etc.); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof.

A guiding component and a nuclease can form a complex (i.e., bind via non-covalent interactions). The guiding component provides target specificity to the complex by comprising a nucleotide sequence that is complementary to a sequence of a target sequence. The nuclease of the complex provides the site-specific activity. In other words, the nuclease is guided to a target sequence (e.g., a target sequence in a chromosomal nucleic acid; a target sequence in an extrachromosomal nucleic acid, e.g., an episomal nucleic acid, a minicircle, etc.; a target sequence in a mitochondrial nucleic acid; a target sequence in a chloroplast nucleic acid; a target sequence in a plasmid; etc.) by virtue of its association with the protein-binding segment (e.g., the interacting portion) of the guiding component.

In some embodiments, the guiding component comprises two separate nucleic acid molecules (e.g., a separate targeting portion and a separate interacting portion; a separate first portion and a separate second portion; or a separate targeting portion-first portion that is covalently bound and a separate second portion). In other embodiments, the guiding component is a single nucleic acid molecule including a covalent bond or a linker between each separate portion (e.g., a targeting portion covalently linked to an interacting portion).

A “host cell,” as used herein, denotes an in vivo or in vitro eukaryotic cell, a prokaryotic cell (e.g., bacterial or archaeal cell), or a cell from a multicellular organism (e.g., a cell line) cultured as a unicellular entity, which eukaryotic or prokaryotic cells can be, or have been, used as recipients for a nucleic acid, and include the progeny of the original cell which has been transformed by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a subject bacterial host cell is a genetically modified bacterial host cell by virtue of introduction into a suitable bacterial host cell of an exogenous nucleic acid (e.g., a plasmid or recombinant expression vector) and a subject eukaryotic host cell is a genetically modified eukaryotic host cell (e.g., a mammalian germ cell), by virtue of introduction into a suitable eukaryotic host cell of an exogenous nucleic acid.

By “linker” is meant any useful multivalent (e.g., bivalent) component useful for joining to different portions or segments. Exemplary linkers include a nucleic acid sequence, a chemical linker, etc. In one instance, the linker of the guiding component (e.g., linker L in the interacting portion of the guiding component) can have a length of from about 3 nucleotides to about 100 nucleotides. For example, the linker can have a length of from about 3 nucleotides (nt) to about 90 nt, from about 3 nucleotides (nt) to about 80 nt, from about 3 nucleotides (nt) to about 70 nt, from about 3 nucleotides (nt) to about 60 nt, from about 3 nucleotides (nt) to about 50 nt, from about 3 nucleotides (nt) to about 40 nt, from about 3 nucleotides (nt) to about 30 nt, from about 3 nucleotides (nt) to about 20 nt or from about 3 nucleotides (nt) to about 10 nt. For example, the linker can have a length of from about 3 nt to about 5 nt, from about 5 nt to about 10 nt, from about 10 nt to about 15 nt, from about 15 nt to about 20 nt, from about 20 nt to about 25 nt, from about 25 nt to about 30 nt, from about 30 nt to about 35 nt, from about 35 nt to about 40 nt, from about 40 nt to about 50 nt, from about 50 nt to about 60 nt, from about 60 nt to about 70 nt, from about 70 nt to about 80 nt, from about 80 nt to about 90 nt, or from about 90 nt to about 100 nt. In some embodiments, the linker of a single-molecule guiding component is 4 nt.

The term “histone-packaged supercoiled plasmid DNA” is used to describe a component of protocells or carriers according to the present disclosure which utilize a plasmid DNA which has been “supercoiled” (i.e., folded in on itself using a supersaturated salt solution or other ionic solution which causes the plasmid to fold in on itself and “supercoil” in order to become more dense for efficient packaging into the protocells or carriers). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells or carriers (either adsorbed into the pores, confined directly within the nanoporous silica core itself, or encapsulated as a biological package). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e., complexing DNA with cationic lipid mixtures). In addition, DNA has also been packaged with cationic proteins (including proteins other than histones), as well as gold nanoparticles (e.g., NanoFlares- an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the disclosure, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present disclosure. The DNA may also be double stranded linear DNA, instead of plasmid DNA, which also may be optionally supercoiled and/or packaged with histones or other packaging components.

Other histone proteins which may be used in this aspect of the disclosure include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44, and HIST4H4.

The term “nuclear localization sequence” refers to a peptide sequence incorporated or otherwise crosslinked into histone proteins which comprise the histone-packaged supercoiled plasmid DNA. In certain embodiments, protocells or carriers according to the present disclosure may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include NH₂-GNQSSNFGPMKGGNFGGRSSGPYGGGGQYFAKPRNQGGYGGC-COOH (SEQ ID NO:9), RRMKWKK (SEQ ID NO:10), PKKKRKV (SEQ ID NO:11), and KR[PAATKKAGQA]KKKK (SEQ ID NO:12), the NLS of nucleoplasmin, a prototypical bipartite signal comprising two clusters of basic amino acids, separated by a spacer of about 10 amino acids. Numerous other nuclear localization sequences are well known in the art. See, for example, LaCasse E C et al., “Nuclear localization signals overlap DNA- or RNA-binding domains in nucleic acid-binding proteins,” Nucl. Acids Res. 1995; 23:1647-56; Weis, K, “Importins and exportins: how to get in and out of the nucleus,” [published erratum appears in Trends Biochem. Sci. 1998 July; 23(7):235] Trends Biochem. Sci. 1998; 23:185-9; and Murat Cokol, Raj Nair & Burkhard Rost, “Finding nuclear localization signals”, at the website ubic.bioc.columbia.edu/papers/2000 nls/paper.html#tab2.

The terms “nucleic acid regulatory sequences,” “control elements,” and “regulatory elements,” used interchangeably herein, refer to transcriptional and translational control sequences, such as promoters, enhancers, polyadenylation signals, internal ribosomal entry sites (IRES), terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

A “promoter sequence” is a DNA regulatory region capable of binding RNA polymerase in a cell and initiating transcription of a downstream (3′ direction) coding sequence. For purposes of defining the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence. Transcriptional and translational control sequences are DNA regulatory sequences, such as promoters, enhancers, polyadenylation signals, terminators, and the like, that provide for the expression of a coding sequence in a host cell.

A “vector” or “expression vector” is a replicon, such as plasmid, phage, virus, or cosmid, to which another nucleic acid segment, i.e., an “insert”, may be attached so as to bring about the replication of the attached segment in a cell.

An “expression cassette” comprises a nucleic acid coding sequence operably linked, as defined herein, to a promoter sequence, as defined herein.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

“Operably linked” or “operatively linked” or “operatively associated with,” as used interchangeably, refers to a juxtaposition wherein the components so described are in a relationship permitting them to function in their intended manner. For instance, a promoter is operably linked to a coding sequence if the promoter affects its transcription or expression. A nucleic acid molecule is operatively linked or operably linked to, or operably associated with, an expression control sequence when the expression control sequence controls and regulates the transcription and translation of nucleic acid sequence. The term “operatively linked” includes having an appropriate start signal (e.g., ATG) in front of the nucleic acid sequence to be expressed and maintaining the correct reading frame to permit expression of the nucleic acid sequence under the control of the expression control sequence and production of the desired product encoded by the nucleic acid sequence. If a gene that one desires to insert into a recombinant DNA molecule does not contain an appropriate start signal, such a start signal can be inserted in front of the gene.

Delivery Platforms

The hallmark of biothreats is genetic novelty that evolves naturally or is introduced deliberately to enhance virulence and multi-drug resistance, rendering existing countermeasures ineffective. To solve this challenge, we have developed a rapid, cost-effective, universal approach to identifying and delivering potent new medical countermeasures against emerging and engineered biological threats. CRISPR can be used to develop novel pathogen- and host-directed countermeasures. CRISPR components can be packaged within state-of-the-art nanoparticle delivery platforms (e.g., protocells or silica carriers), which can be modulated to have useful nanoparticle properties, including size and surface modifications, that promote delivery to specific targets (e.g., organs, cells, pathogens, etc.), uptake by pathogen-infected cells, and release within appropriate intracellular locations (e.g., to achieve targeted cleavage of pathogen DNA or targeted disruption of pathogen-host interactions).

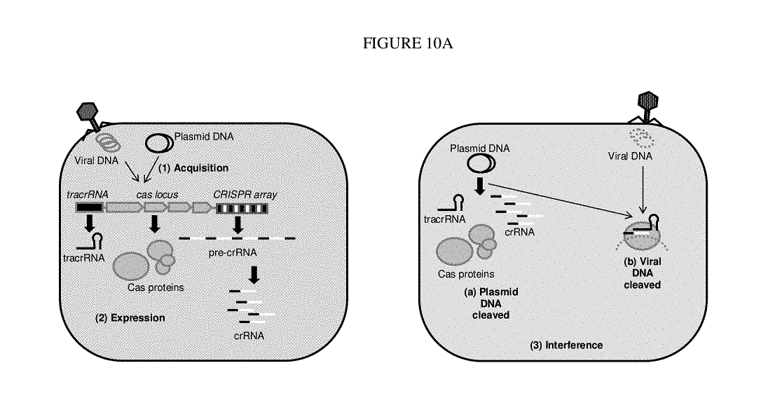

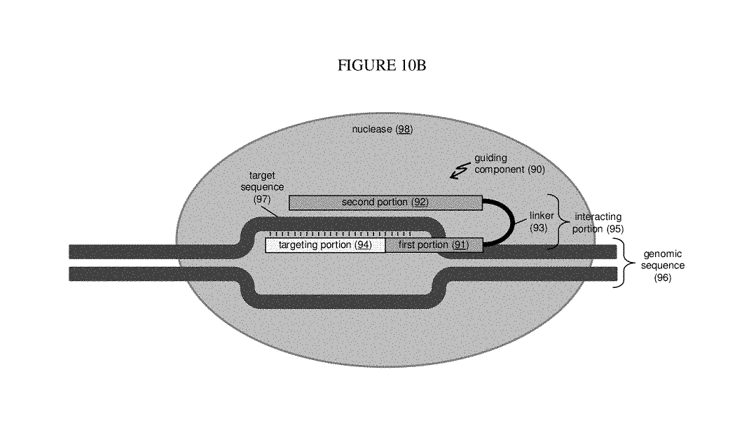

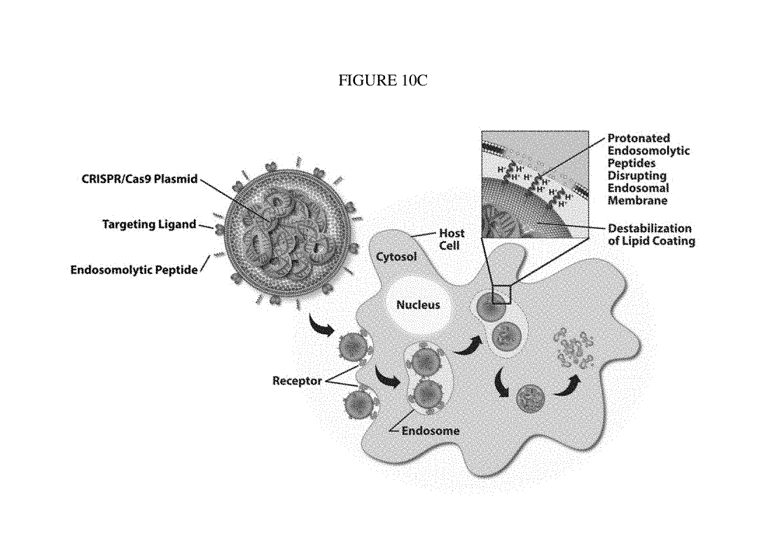

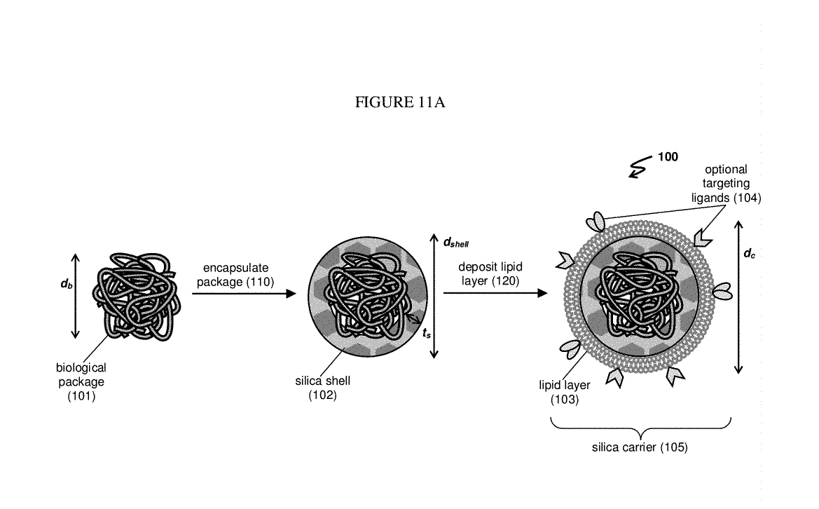

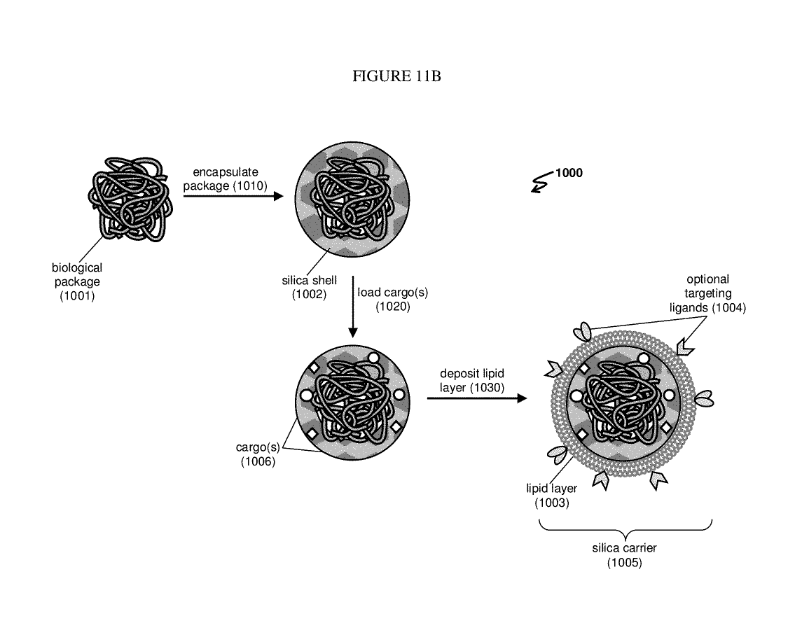

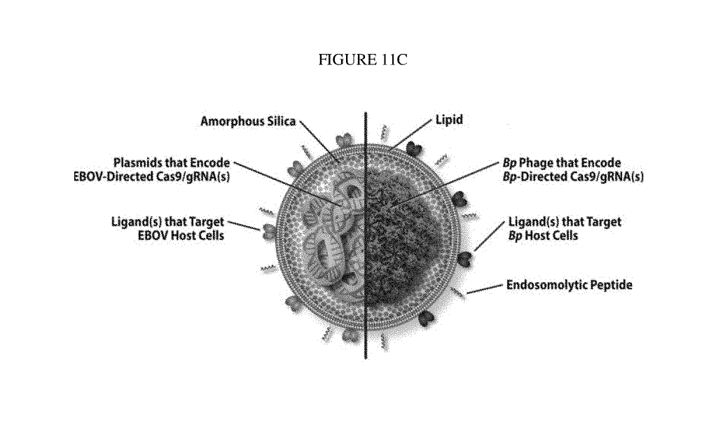

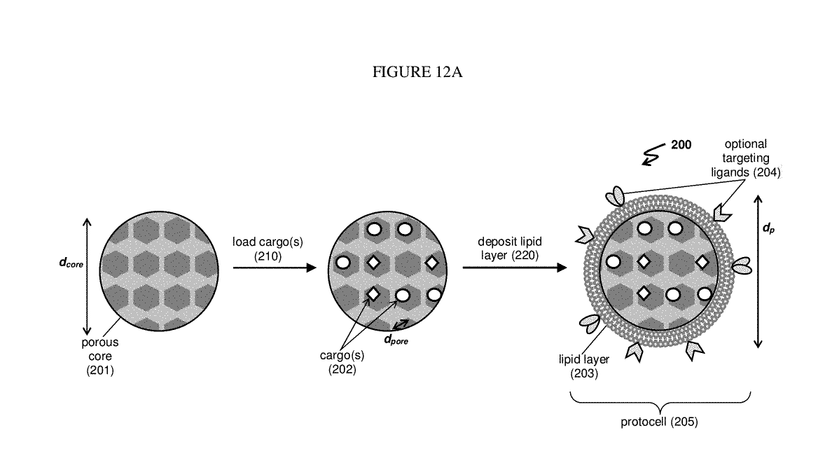

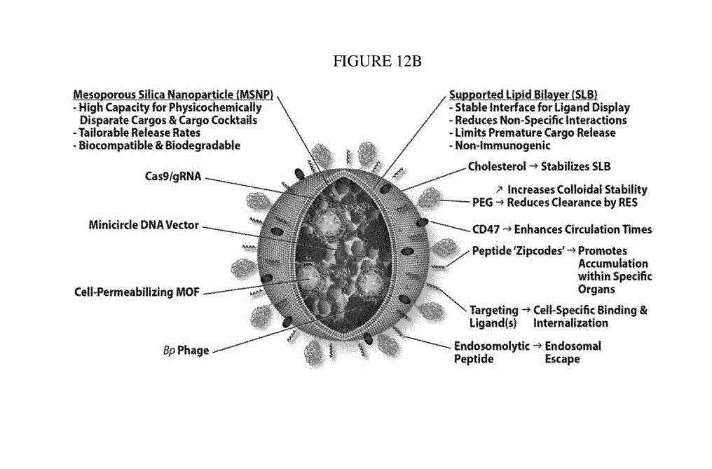

In one instance, the delivery platform includes a CRISPR component, such as a CRISPR/Cas system (e.g., a type II CRISPR/Cas system, as well as modified versions thereof, such as a CRISPR/dCas9 system). Exemplary platforms are shown in FIGS. 11A-11C and 12A-12B, where exemplary CRISPR components are shown in FIGS. 10B, 15, 16A-16H, 17A-17C, 19A-19C, 20, 21, and 22.

The delivery platform (e.g., a NanoCRISPR, as employed herein) can be based on a protocell (e.g., FIG. 12A-12B) or a silica carrier (e.g., FIG. 11A-11C). As described herein, the protocell includes a porous core (e.g., a porous silica core) having one or more cargo deposited within the plurality of pores of the core, whereas the silica carrier includes a silica shell that encapsulates a biological package.

The silica carrier can be formed in any useful manner As seen in the method 100 of FIG. 11A, a biological package 101 having a dimension d_{b} is first provided. Exemplary values for dimension d_{b} include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more). The biological package can include one or more components (e.g., one or more nucleic acid sequences, drugs, proteins, labels, etc., such as any agent described herein).

Then, the biological package 101 is encapsulated 110 with a silica shell 102 having a thickness tₛ, thereby providing a particle of dimension dₛₕₑₗₗ. The shell can have any useful thickness that allows for controlled biodegradation in vivo, targeted biodistribution, stability in a formulation, and/or consistent fabrication of the carrier (or a population of carriers). Exemplary values for dimension is include, without limitation, less than about 100 nm (e.g., less than about 0.1 nm, 0.5 nm, 1 nm, 2 nm, 3 nm, 5 nm, 8 nm, 10 nm, 15 nm, 20 nm, 30 nm, 40 nm, 50 nm, 60 nm, 70 nm, 80 nm, 90 nm). Exemplary values for dimension dₛₕₑₗₗ include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Finally, an optional lipid layer 103 can be deposited 120 on an outer surface of the silica shell (e.g., thereby forming a silica carrier 105). Furthermore, one or more optional targeting ligands 104 (e.g., any described herein) can be combined and/or co-extruded with the lipid and then deposited as a lipid layer (e.g., a lipid bilayer or a lipid multilayer). The silica carrier 105 can have any useful dimension d_{c}. Exemplary values for dimension d_{c} include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Optionally, the method can be adapted to include any other useful component(s) or cargo(s). As seen in the method 1000 of FIG. 11B, a biological package 1001 is encapsulated 1010 with a silica shell 1002. One or more cargos 1006 can be loaded 1020 into the shell (if the shell is porous) or onto the outer surface of the shell (e.g., if the shell is not porous). A lipid layer 1003 can be deposited 1030 on an outer surface of the silica shell (e.g., thereby forming a silica carrier 1005). Furthermore, one or more optional targeting ligands 1004 can be present in the lipid layer 1003.

FIG. 11C provides an exemplary, non-limiting silica carrier having a silica shell that encapsulates a plasmid that targets a viral genomic sequence (e.g., by way of a CRISPR component that targets Ebola virus) or a phage that target a bacterial genomic sequence (e.g., by way of a CRISPR component that targets Bp). The carrier can be optimized to include surface ligands that specifically target the desired cell or pathogen.

The protocell can be formed in any useful manner As seen in the method 200 of FIG. 12A, a porous core 201 having a dimension d_{core} is first provided. Exemplary values for dimension d_{core} include, without limitation, greater than about 1 nm (e.g., greater than about 5 nm, 10 nm, 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

Then, one or more cargos 202 are loaded 210 into the pores of core, in which the pore has a dimension dₚₒᵣₑ. Exemplary values for dimension dₚₒᵣₑ include, without limitation, greater than about 0.5 nm (e.g., around 0.5 nm to about 25 nm in diameter, often about 1 to around 20 nm in diameter).

A lipid layer 203 can be deposited 220 on an outer surface of the core (e.g., thereby forming a protocell 205). Furthermore, one or more optional targeting ligands 204 can be present in the lipid layer 203. The protocell can have any useful dimension, such as a diameter dₚ. Exemplary values for dimension dₚ include, without limitation, greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 μm, 2 μm, 5 μm, 10 μm, 20 μm, or more).

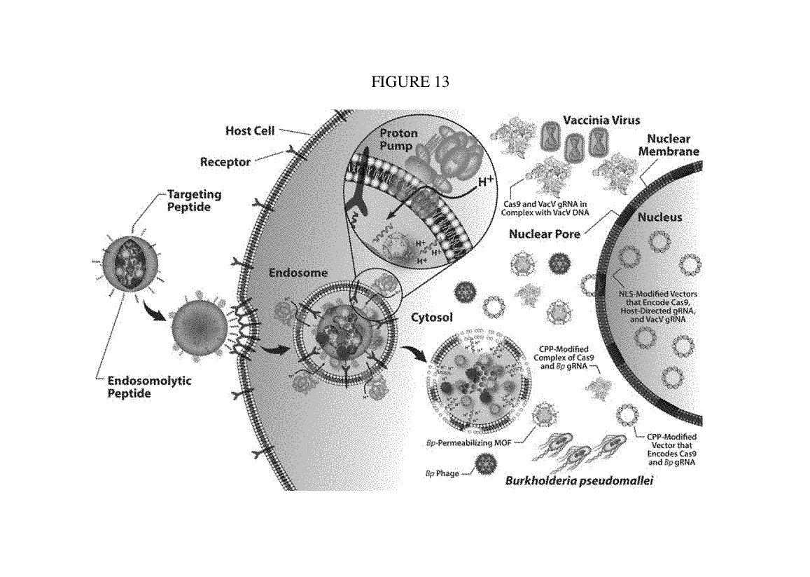

FIG. 12B provides an exemplary, non-limiting protocell containing cargo within pores or associating with cargo on an outer surface of the core for the protocell. For instance, the cargo can include a CRISPR component (e.g., Cas9/gRNA complex), vectors, metal-organic framework (if needed), and a phage that target a bacterial genomic sequence (e.g., by way of a CRISPR component that targets Bp). The carrier can be optimized to include surface ligands that specifically target the desired cell or pathogen. FIG. 13 provides a non-limiting schematic of use of the protocell including a CRISPR component (e.g., an exemplary NanoCRISPR) to target viruses and bacteria in a host cell.

As can be seen, additional components may be present in the delivery platform. In one instance, the delivery platform includes one or more components that facilitate CRISPR delivery to the target, such as modified CRISPR components with cell-penetrating peptides, co-delivery of CRISPR components with metal organic frameworks (MOFs) designed to permeabilize bacteria, and/or use of phage that encode CRISPR components. Additional details on the protocell, the silica carrier, the CRISPR/Cas system, biological package, and cargo are described herein.

Characteristics of the Delivery Platform

A protocell generally includes a porous core and a supported lipid layer (e.g., a supported lipid bilayer (SLB)). In one instance, the core is a mesoporous silica nanoparticle (MSNP). In another instance, the core optionally includes a cell-permeabilizing metal organic framework. One or more cargoes can be disposed within a plurality of pores of the core. Optionally, cargo(s) can be linked to the SLB (e.g., by a linker, such as any described herein).

A silica carrier generally includes a biological package encapsulated in a silica shell and can optionally include a supported lipid layer (e.g., a supported lipid bilayer or supported lipid multilayer having more than three lipid layers). One or more cargoes can be disposed within the silica shell and/or with the biological package within the shell.

The particle size distribution (e.g., size of the core for the protocell or the silica carrier), according to the present disclosure, depends on the application, but is principally monodisperse (e.g., a uniform sized population varying no more than about 5-20% in diameter, as otherwise described herein). In certain embodiments, particles can range, e.g., from around 1 nm to around 500 nm in size, including all integers and ranges there between. The size is measured as the longest axis of the particle. In various embodiments, the particles are from around 5 nm to around 500 nm and from around 10 nm to around 100 nm in size.

The particles can have a porous structure (e.g., as a core or as a shell). The pores can be from around 0.5 nm to about 25 nm in diameter, often about 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

In certain embodiments, preferred MSNPs, protocells, or carriers according to the present disclosure: are monodisperse and range in size from about 25 nm to about 300 nm; exhibit stability (colloidal stability); have single cell binding specification to the substantial exclusion of non-targeted cells; are anionic, neutral or cationic for specific targeting (preferably cationic); are optionally modified with agents such as PE1, NMe³⁺, dye, crosslinker, ligands (ligands provide neutral charge); and optionally, are used in combination with a cargo to be delivered to the target.

In certain alternative embodiments, the MSNPs, protocells, or carriers are monodisperse and range in size from about 25 nm to about 300 nm. The sizes used preferably include 50 nm (+/−10 nm) and 150 nm (+/−15 nm), within a narrow monodisperse range, but may be more narrow in range.

In certain alternative embodiments, the present disclosure are directed to MSNPs and preferably, protocells, or carriers of a particular size (diameter) ranging from about 0.5 to about 30 nm, about 1 nm to about 30 nm, often about 5 nm to about 25 nm (preferably, less than about 25 nm), often about 10 to about 20 nm, for administration via intravenous, intramuscular, intraperitoneal, retro-orbital and subcutaneous injection routes. These MSNPs, protocells, and/or carriers are often monodisperse and provide colloidally stable compositions. These compositions can be used to target tissues in a patient or subject because of enhanced biodistribution/bioavailability of these compositions, and optionally, specific cells, with a wide variety of therapeutic and/or diagnostic agents which exhibit varying release rates at the site of activity.

The particles (e.g., having a core or a shell) can be produced in any useful manner In one instance, particles with 7.9 nm pores (e.g., in the core or in the shell) can be prepared with templating by Pluronic® F127. In another instance, the particles include 18-25 nm pores (see, e.g., Gao F et al., J. Phys. Chem. B. 2009; 113:1796-804). In yet another instance, the pores can be templated with cross-linked micelles, thereby providing pores with precise diameters ranging from 10 nm to 20 nm. Various sizes of cross-linked micelles will be prepared by mixing various concentrations of Pluronic® F127 with polypropylene oxide, 25% tetrahydrofuran, and benzoyl peroxide; the resulting micelle solution will then be aged for 24 hours at 60° C., vacuum dried, and added to the silica precursor solution. Each batch of particles can be characterized in any useful manner, such as by assessment of size and surface charge using dynamic light scattering (DLS) (NIST-NCL PCC-1 and PCC-2) and electron microscopy (NIST-NCL PCC-7 and PCC-15) and verification of low endotoxin contamination per health industry product standards (NCL STE-1.1). In addition, ten percent of particle (e.g., NanoCRISPR) batches will be randomly tested for solvent and surfactant contamination using mass spectrometry.

To enable burst release of CRISPR components (e.g., guiding component(s) and nuclease component(s), including the nuclease or a nucleic acid sequence that encodes the nuclease) in the cytosol of host cells, pore-templating surfactants and cross-linked micelles can be extracted (e.g., using acidified ethanol to minimize the degree of silica condensation in the particle framework). Furthermore, if the cargo has an isoelectric points or pKa values <7, then naturally negatively-charged particles can be modified with amine-containing silanes (e.g., (3-aminopropyl)triethoxysilane, or APTES) in order to maximize electrostatic interactions between pore walls and cargo molecules.

The core of a protocell can be loaded in any useful manner For instance, loading with CRISPR components, alone and in combination with small molecule antimicrobials, can be accomplished by soaking the MSNP with the cargo (see, e.g., Ashley C E et al., ACS Nano 2012; 6:2174-88; Ashley C E et al., Nat. Mater. 2011; 10: 389-97; and Epler K et al., Adv. Healthc. Mater. 2012 1:348-53). Loading capacities for Cas9/guiding component complexes and other agents (e.g., small molecule antimicrobials and/or antivirals) can be determined in any useful manner (e.g., using spectrophotometer and absorbance or fluorescence-based HPLC methods). Release rates can be confirmed upon encapsulation of cargo-loaded MSNPs in an SLB (e.g., a DOPC SLB) and dispersion in simulated body and/or endolysosomal fluids.

Pore size of the core can be modified, as needed, to accommodate the CRISPR components, as well as any other cargo. We have previously shown that MSNPs with 18-25 nm pores can be loaded with high concentrations of minicircle DNA vectors up to 2000-bp in size, as well as histone-packaged plasmids up to 6000-bp in size via our simple soaking procedure (see e.g., Ashley C E et al., ACS Nano 2012; 6:2174-88; Ashley C E et al., Nat. Mater. 2011; 10: 389-97; and Epler K et al., Adv. Healthc. Mater. 2012 1:348-53). To minimize possible anti-histone antibody responses in vivo (e.g., arising from pre-packaged plasmids within the core), the cargo can be entrapped within the MSNPS as they are being formed in EISA reactors. Such cargo can include any herein, such as linear and circular DNA vectors of various sizes.

Alternatively, CRISPR components can be encapsulated within a silica shell, as in a silica carrier. In this configuration, large CRISPR components (e.g., having a dimension greater than about 20 nm or having more than about 6,000-bp) can be obtained, and the biodegradable silica shell can be built around the CRISPR component(s). In this manner, self-assembly processes provide no limit as to the size of the biological package that can be encapsulated in the silica shell. Of course, carrier size can affect biodistribution and cellular uptake, which can be controlled in the manner described herein.

Cargo can be introduced to the core in any useful manner For instance, the cargo can be introduced (e.g., by soaking) after the MSNP is synthesized. Alternatively, cargo can be introduced during MSNP or silica shell synthesis. In yet another instance, cargo is complexed with the biological package prior to encapsulation with a silica shell. In another instance, the cargo is introduced (e.g., by soaking) after the silica shell of the carrier is synthesized.

In one instance, cargo can be introduced at various concentrations into the precursor solution, which will then aerosolize and pass through the reactor at high flow rates to minimize exposure of the cargo to high temperatures (e.g., <1 second in the 400° C. heating zone). Within each aerosolized droplet, silica will self-assemble around the cargo (e.g., DNA molecules), resulting in nanoparticles that entrap the cargo. For a cargo being DNA, preliminary experiments indicate we can entrap ˜0.3 mg of a 3300 bp DNA vector per mg of MSNPs and that, upon dissolution of the silica framework, the DNA vector, which encodes expression of a fluorescent reporter protein (ZsGreen), is able to transfect Vero cells. These data indicate that the process does not damage the vector. Similar methodologies can be employed to entrap any useful agent, such as a cargo (e.g., phage) or a MOF.

Co-loading of cargos can also be implemented in any useful manner For instance, to enable co-loading of DNA- and phage-based countermeasures with small molecule antimicrobials, cetyltrimethylammonium bromide (CTAB) can be employed in the precursor solution to template 2.5 nm pores in resulting MSNPs. Then, CTAB can be extracted using acidified ethanol to promote burst release rates.

CRISPR/Cas Components

The present disclosure relates to a delivery platform including one or more CRISPR components (e.g., associated with the core, within the shell, and/or the supported lipid bilayer). FIG. 10A-10C shows a CRISPR component and its non-limiting use with a delivery platform described herein. The CRISPR/Cas system evolved naturally within prokaryotes to confer resistance to exogenous genetic sequences (FIG. 10A-10B). As can be seen (FIG. 10A), the CRISPR/Cas system can include a CRISPR array that is a noncoding RNA transcript that is further cleaved into CRISPR RNA (crRNA), a trans-acting CRISPR RNA (tracrRNA), and various CRISPR-associated (Cas) proteins.

This CRISPR/Cas system can be adapted to control genetic expression in targeted manner, such as, e.g., by employing synthetic, non-naturally occurring constructs that use crRNA nucleic acid sequences, tracrRNA nucleic acid sequences, and/or Cas polypeptide sequences, as well as modified forms thereof.

One CRISPR component includes a guiding component. In general, the guiding component includes a nucleic acid sequence (e.g., a single polynucleotide) that includes at least two portions: (1) a targeting portion, which is a nucleic acid sequence that imparts specific targeting to the target genomic locus (e.g., a guide RNA or gRNA); and an interacting portion, which is another nucleic acid sequence that binds to a nuclease (e.g., a Cas endonuclease). In some instances, the interacting portion includes two particular sequences that bind the nuclease, e.g., (2) a short crRNA sequence attached to the guide nucleic acid sequence; and (3) a tracrRNA sequence attached to the crRNA sequence. Exemplary targeting CRISPR components include a minicircle DNA vector optimized for in vivo expression.

Another CRISPR component includes a nuclease (e.g., that binds the targeting nucleic acid sequence). The nuclease CRISPR component can either be an enzyme, or a nucleic acid sequence that encodes for that enzyme. Exogenous endonuclease (e.g., Cas9) can be encoded by a cargo stored within the protocell and/or the silica carrier. Any useful nuclease can be employed, such as Cas9 (e.g., SEQ ID NO:110), as well as nickase forms and deactivated forms (e.g., SEQ ID NO:111) thereof (e.g, including one or more mutations, such as D10A, H840A, N854A, and N863A in SEQ ID NO:110 or in an amino acid sequence sufficiently aligned with SEQ ID NO:110), including nucleic acid sequences that encode for such nuclease. Pathogen-directed and host-directed CRISPR components (e.g., guiding components and/or nuclease), as well as minicircle DNA vectors that encode Cas and guiding components can be developed.

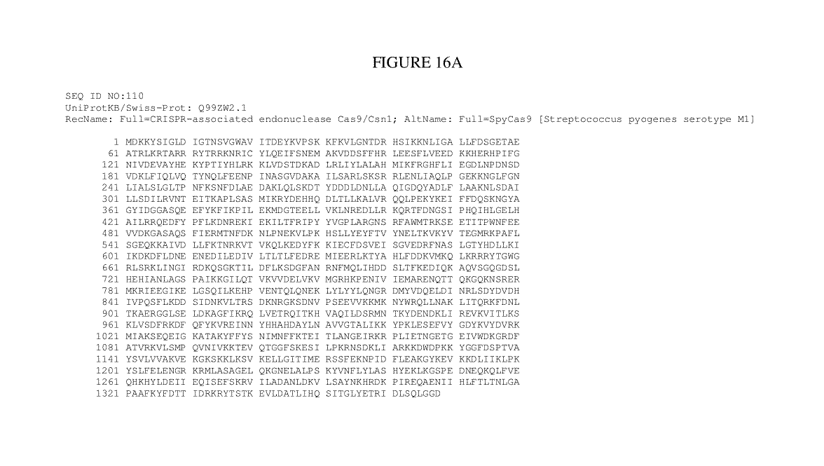

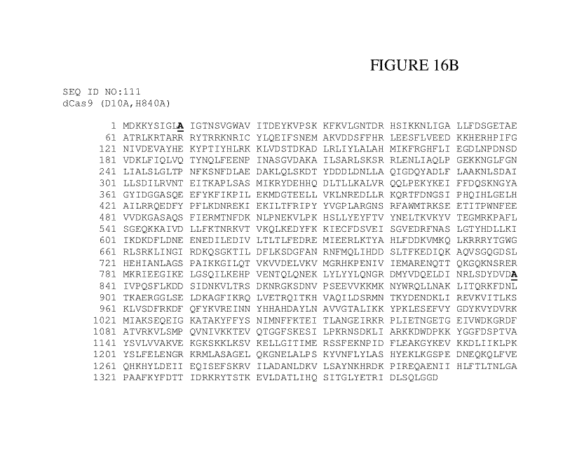

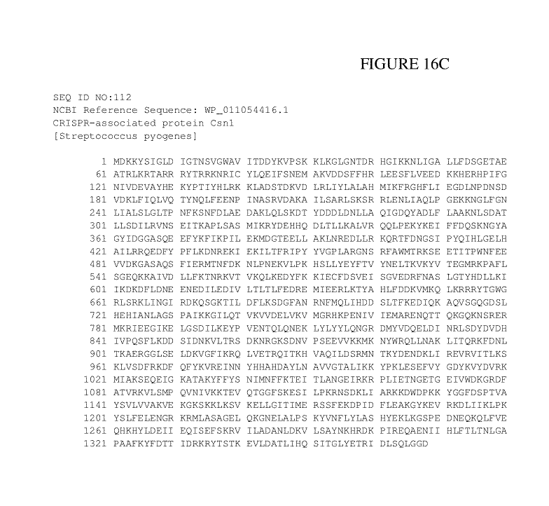

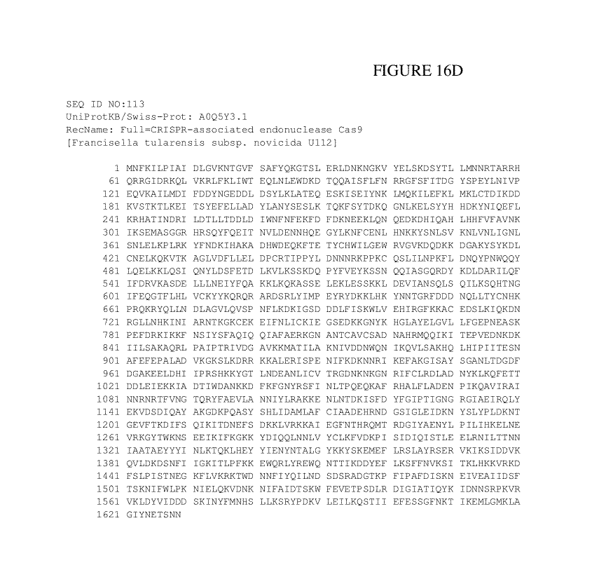

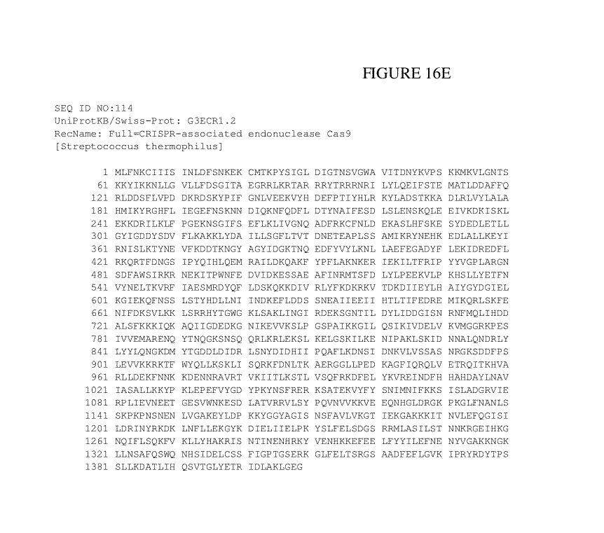

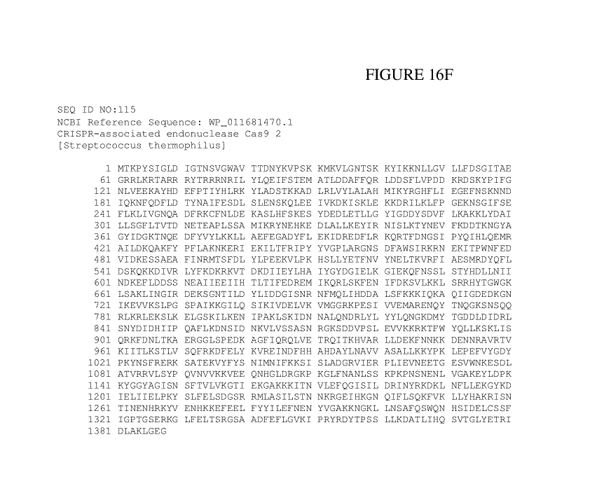

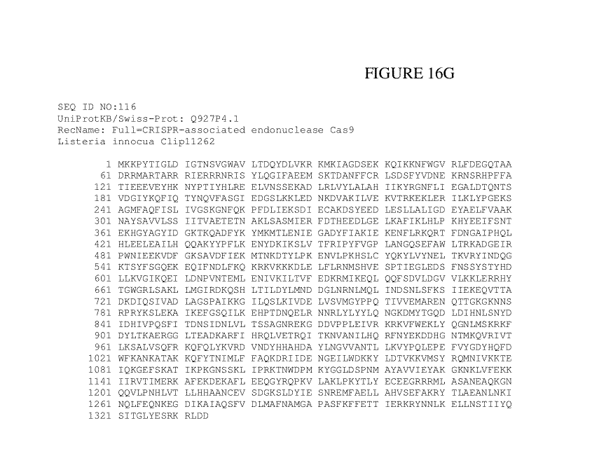

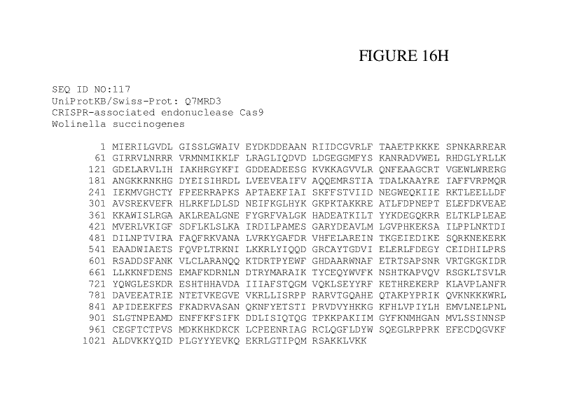

Non-limiting examples of nucleases are described in FIG. 16A-16H. In some embodiments, a vector comprises a regulatory element operably linked to an enzyme-coding sequence encoding a nuclease (e.g., a CRISPR enzyme, such as a Cas protein). Non-limiting examples of Cas proteins include Casl, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence.

The nuclease may be a Cas9 homolog or ortholog. In some embodiments, the nuclease is codon-optimized for expression in a eukaryotic cell. In some embodiments, the nuclease directs cleavage of one or two strands at the location of the target sequence. In some embodiments, the nuclease lacks DNA strand cleavage activity. In some embodiments, the first regulatory element is a polymerase III promoter. In some embodiments, the second regulatory element is a polymerase II promoter.

Any useful Cas protein or complex can be employed. Exemplary Cas proteins or complexes include those involved in Type I, Type II, or Type III CRISPR/Cas systems, including but not limited to the CRISPR-associated complex for antiviral defence (Cascade, including a RAMP protein), Cas3 and/or Cas 7 (e.g., for Type I systems, such as Type I-E systems), Cas9 (formerly known as Csn1 or Csx12, e.g., such as in Type II systems), Csm (e.g., in Type III-A systems), Cmr (e.g., in Type III-B systems), Cas10 (e.g., in Type III systems), as well as subassemblies or sub-components thereof and assemblies including such Cas proteins or complexes. Additional Cas proteins and complexes are described in Makarova K S et al., “Evolution and classification of the CRISPR—Cas systems,” Nat. Rev. Microbiol. 2011; 9:467-77, which is incorporated herein by reference in its entirety.

In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the disclosure, nickases may be used for genome editing via homologous recombination.

As a further example, two or more catalytic domains of Cas9 (RuvC I, RuvC II, and RuvC III) may be mutated to produce a mutated Cas9 substantially lacking all DNA cleavage activity. In some embodiments, a D10A mutation is combined with one or more of H840A, N854A, or N863A mutations to produce a Cas9 enzyme substantially lacking all DNA cleavage activity. In some embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes , mutations in corresponding amino acids may be made to achieve similar effects.

FIG. 10B shows an exemplary CRISPR component that includes a guiding component 90 to bind to the target sequence 97, as well as a nuclease 98 (e.g., a Cas nuclease or an endonuclease, such as a Cas endonuclease) that interacts with the guiding component and the target sequence. As can be seen, the guiding component 90 includes a targeting portion 94 configured to bind to the target sequence 97 of a genomic sequence 96 (e.g., a target sequence having substantially complementarity with the genomic sequence or a portion thereof). In this manner, the targeting portion confers specificity to the guiding component, thereby allowing certain target sequences to be activated, inactivated, and/or modified.

The guiding component 90 also includes an interacting portion 95, which in turn is composed of a first portion 91, a second portion 92, and a linker 93 that covalently links the first and second portions. The interacting portion 95 is configured to recruit the nuclease (e.g., a Cas nuclease) in proximity to the site of the target sequence. Thus, the interacting portion includes nucleic acid sequences that provide preferential binding (e.g., specific binding) of the nuclease. Once in proximity, the nuclease 98 can bind and/or cleave the target sequence or a sequence in proximity to the target sequence in a site-specific manner

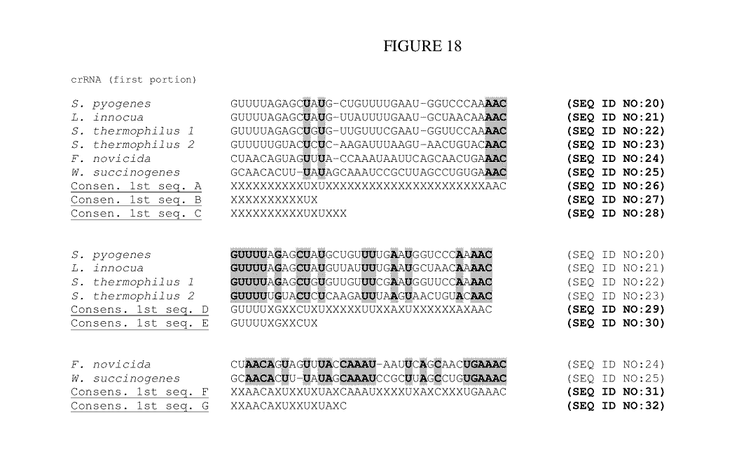

The first portion, second portion, and linker can be derived in any useful manner. In one instance, the first portion can include a crRNA sequence, a consensus sequence derived from known crRNA sequences, a modified crRNA sequence, or an entirely synthetic sequence known to bind a Cas nuclease or determined to competitively bind a Cas nuclease when compared to a known crRNA sequence. Exemplary sequences for a first portion are described in FIG. 18 (SEQ ID NOs:20-32). In some embodiments, the first portion is a crRNA sequence that exhibits at least 50%, 60%, 70%, 80%, 90%, 95%, or 99% of sequence complementarity to any one of SEQ ID NOs:20-32. In other embodiments, the first portion is a fragment (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more nucleotides) of a crRNA sequence that exhibits at least 50%, 60%, 70%, 80%, 90%, 95%, or 99% of sequence complementarity to any one of SEQ ID NOs:20-32.

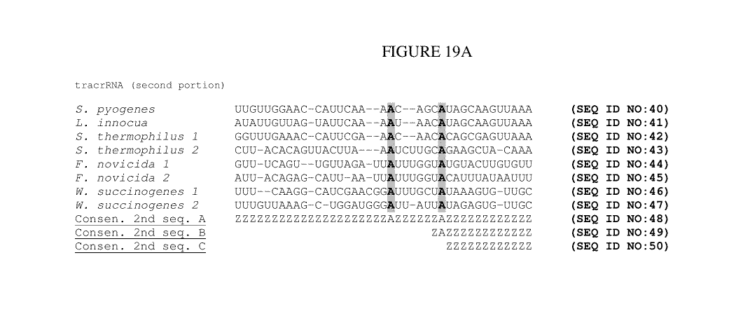

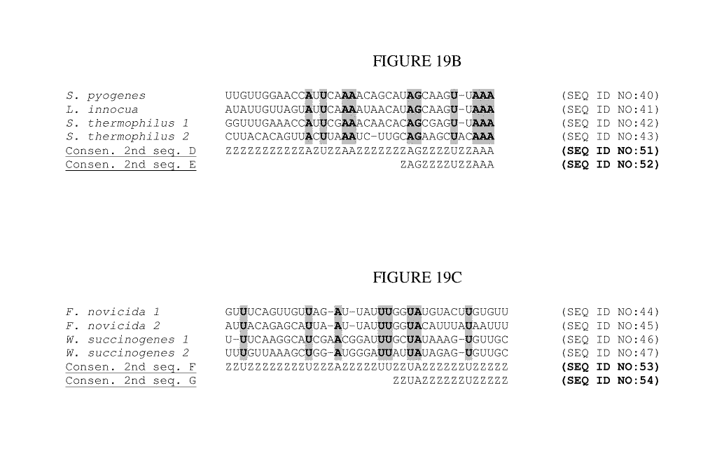

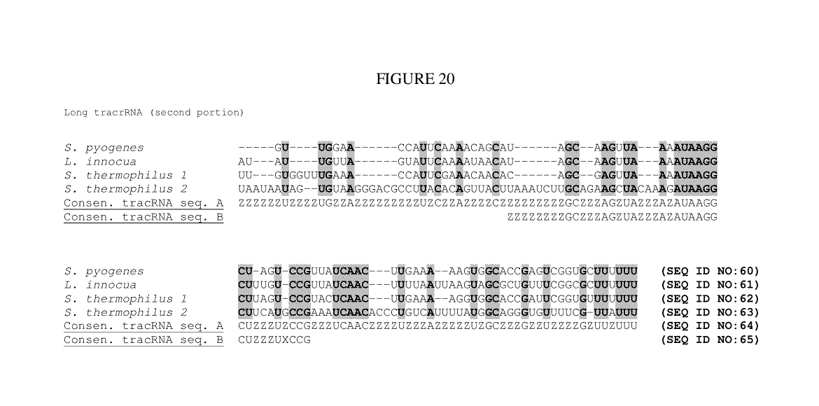

In another instance, the second portion can include a tracrRNA sequence, a consensus sequence derived from known tracrRNA sequences, a modified tracrRNA sequence, or an entirely synthetic sequence known to bind a Cas nuclease or determined to competitively bind a Cas nuclease when compared to a known tracrRNA sequence. Exemplary sequences for a second portion are described in FIG. 19A-19C (SEQ ID NOs:40-54) and in FIG. 20 (SEQ ID NOs:60-65). In some embodiments, the second portion is a tracrRNA sequence that exhibits at least 50%, 60%, 70%, 80%, 90%, 95%, or 99% of sequence complementarity to any one of SEQ ID NOs:40-54 and 60-65. In other embodiments, the second portion is a fragment (e.g., having a length of about 4, 5, 6, 7, 8, 9, 10, 11, 12, 13, 14, 15, 16, 17, 18, 19, 20, 22, 24, 26, 28, 30, 32, 34, 36, 38, 40, or more nucleotides) of a tracrRNA sequence that exhibits at least 50%, 60%, 70%, 80%, 90%, 95%, or 99% of sequence complementarity to any one of SEQ ID NOs:40-54 and 60-65.

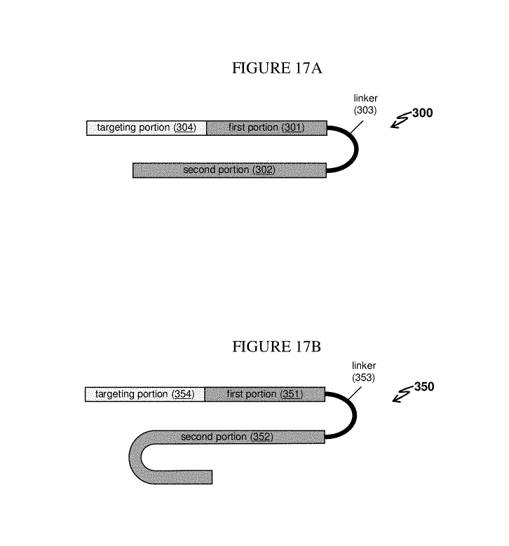

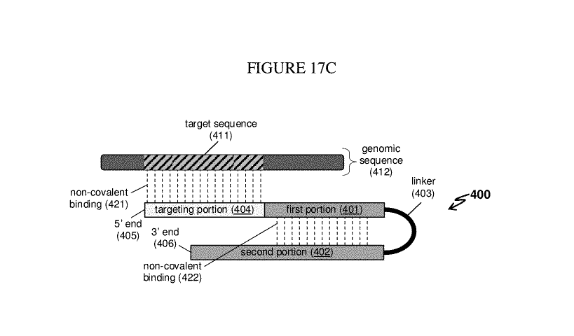

The linker can be any useful linker (e.g., including one or more transcribable elements, such as a nucleotide or a nucleic acid, or including one or more chemical linkers). Further, the linker can be derived from a fragment of any useful tracrRNA sequence (e.g., any described herein). The first and second portions can interact in any useful manner For example, the first portion can have a sequence portion that is sufficiently complementary to a sequence portion of the second portion, thereby facilitating duplex formation or non-covalent bonding between the first and second portion. In another example, the second portion can include a first sequence portion that is sufficiently complementary to a second sequence portion, thereby facilitating hairpin formation within the second portion. Further CRISPR components are described in FIG. 17A-17C.

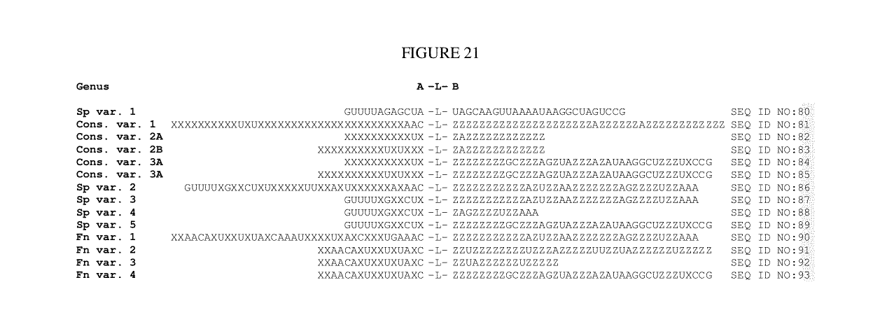

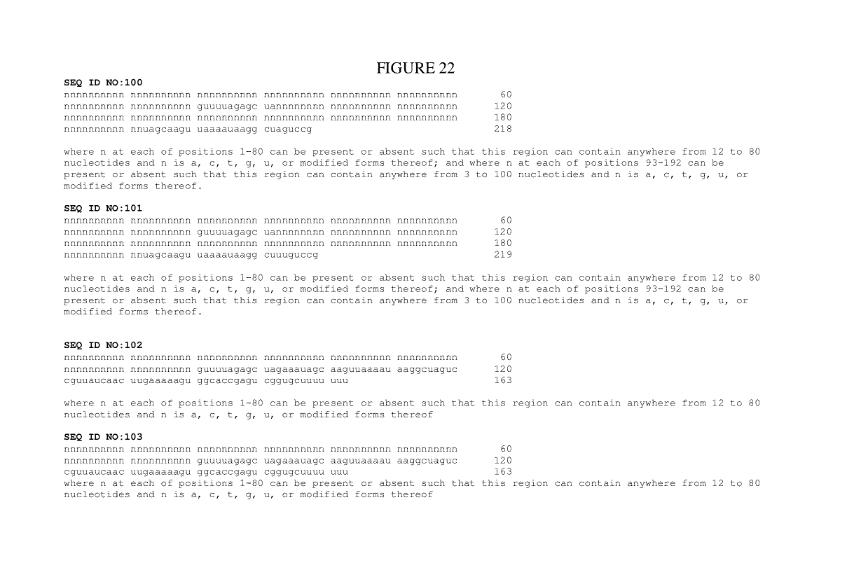

In another embodiment, the guiding component has a structure of A-L-B, in which A includes a first portion (e.g., any one of SEQ ID NOs:20-32, or a fragment thereof), L is a linker (e.g., a covalent bond, a nucleic acid sequence, a fragment of any one of SEQ ID NOs:40-54 and 60-65, or any other useful linker), and B is a second portion (e.g., any one of SEQ ID NOs:40-54 and 60-65, or a fragment thereof) (FIG. 21). In yet another embodiment, the guiding component is a sequence that exhibits at least 50%, 60%, 70%, 80%, 90%, 95%, or 99% of sequence complementarity to any one SEQ ID NOs:100-103, or a fragment thereof (FIG. 22).

FIG. 10C shows delivery of a CRISPR component (e.g., as a plasmid) by employing a silica carrier. The CRISPR components can be provided in any useful form (e.g., a vector for in vivo expression, a phage, a plasmid, etc.). In some embodiments, the CRISPR component includes ds plasmid DNA, which is modified to express RNA and/or a protein. In other embodiments, the CRISPR component is supercoiled and/or packaged (e.g., within a complex, such as those containing histones, lipids (e.g., lipoplexes), proteins (e.g., cationic proteins), cationic carrier, nanoparticles (e.g., gold or metal nanoparticles), etc.), which may be optionally modified with a nuclear localization sequence (e.g., a peptide sequence incorporated or otherwise crosslinked into histone proteins, which comprise the histone-packaged supercoiled plasmid DNA). Other exemplary histone proteins include H1, H2A, H2B, H3 and H4, e.g., in a ratio of 1:2:2:2:2. Exemplary nuclear localization sequences include H₂N-GNQSSNFGPMKGGNFGGRSSGPYGGGGQYFAKPRNQGGYGGC-COOH (SEQ ID NO:9), RRMKWKK (SEQ ID NO:10), PKKKRKV (SEQ ID NO:11), and KR[PAATKKAGQA]KKKK (SEQ ID NO:12), the NLS of nucleoplasmin, a prototypical bipartite signal comprising two clusters of basic amino acids, separated by a spacer of about 10 amino acids, as well as any described in LaCasse E C et al., Nucleic Acids Res. 1995 May 25; 23(10):1647-56; Weis K, Trends Biochem. Sci. 1998 May; 23(5):185-9; and Cokol M et al., EMBO Rep. 2000 Nov. 15; 1(5): 411-5, each of which is incorporated herein by reference in its entirety.

The CRISPR component can include any useful promoter sequence(s), expression control sequence(s) that controls and regulates the transcription and translation of another DNA sequence, and signal sequence(s) that encodes a signal peptide. The promoter sequence can include a DNA regulatory region capable of binding RNA polymerase in a cell and initiating transcription of a downstream (3′ direction) coding sequence. For purposes of defining the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation, as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgamo sequences in addition to the −10 and −35 consensus sequences.

In addition, the CRISPR components can be formed from any useful combination of one or more nucleic acids (or a polymer of nucleic acids, such as a polynucleotide). Exemplary nucleic acids or polynucleotides of the disclosure include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids, chimeras, or modified forms thereof. Exemplary modifications include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present disclosure may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

Toxicity of CRISPR components, to the host, can be minimized in any useful manner. For instance, toxicity can result from protocells or carriers due to expression of Cas9 products or immune responses. Specifically, the lifetime of CRISPR components in the cell can be controlled by adding features that are stabilized or destabilized with cellular proteases, by inducing expression only under a microbial or viral promoter, and by using guiding components with modified backbones (e.g., 2-OMe) to minimize immune recognition.

Resistance to CRISPR components can be minimized Any single antibiotic or antiviral countermeasure is prone to the development of resistance, so pathogens will likely mutate around individual guiding component targets. However, we will prevent the development of resistance by targeting orthogonal mechanisms via multiplexed guiding components in combination with current antivirals/antimicrobials.

Off-target mutations or genetic modification can be minimized. For instance, bioinformatic guiding component design programs can be used to determine minimal effective CRISPR component doses. If needed, the nickase version of Cas9 can be employed. More specifically, Cas9 targeted against a virus will not enter the nucleus of the host cell, phage-delivered CRISPR components will not be expressed in mammalian cells, plasmids that encode antibacterial CRISPR components will be under a bacterial promoter, and host-directed therapies will only bind host DNA, not induce cleavage. Together, these methods should reduce if not eliminate off-target effects.

The CRISPR component can be employed to target any useful nucleic acid sequence (e.g., present in the host's genomic sequence and/or the pathogen's genomic sequence). In one instance, the target sequence can include a sequence present in the host's genomic sequence in order, e.g., activate, inactive, or modify expression of factor or proteins within the host's cellular machinery. For instance, the target sequence can bind to one or more genomic sequences for an immunostimulatory protein that, upon expression, would enhance the immune response by the host to an infection. Pathogens are known to down-regulate proteins that would otherwise assist in recognizing non-self protein motifs. Thus, in another instance, the target sequence can bind to one or more regulator proteins and enhance their transcription and expression. In yet another instance, one or more polypeptides may be up-regulated, as compared to the normal basal rate, and such up-regulation may be modified by the presence of the pathogen. Accordingly, the target sequence can be employed to bind to one or more up-regulated polypeptides in order to inactivate or repress transcription/expression of those polypeptides.

Exemplary target sequence (e.g., in a host or subject) includes, without limitation, a nucleic acid sequence encoding an immunostimulatory protein, a cluster of differentiation protein, a cell surface protein, a pathogen receptor protein (e.g., a pathogen recognition receptor, such as TLR9), a glycoprotein (e.g., granulocyte-colony stimulating factor), a cytokine (e.g., interferon or transforming growth factor beta (TGF-beta)), a pattern recognition receptor protein, a hormone (e.g., a prostaglandin), or a helicase enzyme.

In yet another instance, the target sequence can be employed to activate, inhibit, and/or modify a target sequence (e.g., associated with the presence of a pathogen, a tumor, etc.). For instance, the target sequence can be configured to activate one or more target sequences encoding proteins that promote programmed cell death or apoptosis (e.g., of the pathogen or of particular tissue types, such as metastatic growths, tumors, lesions, etc.). For instance, the target sequence can be configured to inactivate or modify one or more target sequences encoding proteins that are suppressed by the pathogen. Exemplary target sequence (e.g., in a pathogen) includes, without limitation, a nucleic acid sequence encoding a virulence factor (e.g., a lipase, a protease, a nuclease (e.g., a DNAse or an RNase), a hemolysin, a hyaluronidase, an immunoglobulin protease, an endotoxin, or an exotoxin), a cell surface protein (e.g., an adhesion), an envelope protein (e.g., a phospholipid, a lipopolysaccharide, a lipoprotein, or a polysaccharide), a glycoprotein, a polysaccharide protein, a transmembrane protein (e.g., an invasin), or a regulatory protein.

The CRISPR component can be employed to activate the target sequence (e.g., the Cas polypeptide can include one or more transcriptional activation domains, which upon binding of the Cas polypeptide to the target sequence, results in enhanced transcription and/or expression of the target sequence), inactivate the target sequence (e.g., the Cas polypeptide can bind to the target sequence, thereby inhibiting expression of one or more proteins encoded by the target sequence; the Cas polypeptide can introduce double-stranded or single-stranded breaks in the target sequence, thereby inactivating the gene; or the Cas polypeptide can include one or more transcriptional repressor domains, which upon binding of the Cas polypeptide to the target sequence, results in reduced transcription and/or expression of the target sequence), and/or modify the target sequence (e.g., the Cas polypeptide can cleave the target sequence of the pathogen and optionally inserts a further nucleic acid sequence).

Any useful transcriptional activation domains can be employed (e.g., VP64, VP16, HIV TAT, or a p65 subunit of nuclear factor KB). In particular, such activation domains are useful when employed with a deactivated or modified form of the Cas polypeptide with minimized cleavage activity. In this way, specific recruitment of the Cas polypeptide to the target sequence is enabled by the interacting portion of the target component, and transcriptional activity is controlled by the activation domains.

Further, any useful transcriptional repressor domains can be employed (e.g., a Krüppel-associated box domain, a SID domain, an Engrailed repression domain (EnR), or a SID4X domain). In particular, such repressor domains can be employed with a deactivated or modified form of the Cas polypeptide with minimized cleavage activity or with an active Cas polypeptide with retained endonuclease activity.

A guiding component may be selected to target any target sequence. In some embodiments, the target sequence is a sequence within a genome of a host (e.g., a host cell) or a pathogen (e.g., a pathogen cell). In some embodiments, the guiding component is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guiding component is less than about 75, 50, 45, 40, 35, 30, 25, 20, 15, 12, or fewer nucleotides in length. The ability of a guiding component to direct sequence-specific binding of a CRISPR complex to a target sequence may be assessed by any suitable assay. For example, the components of a CRISPR system sufficient to form a CRISPR complex, including the guiding component to be tested, may be provided to a host cell having the corresponding target sequence, such as by transfection with vectors encoding the components of the CRISPR sequence, followed by an assessment of preferential cleavage within the target sequence, such as by Surveyor assay. Similarly, cleavage of a target sequence may be evaluated in a test tube by providing the target sequence, components of a CRISPR complex, including the guiding component to be tested and a control guiding component different from the test guiding component, and comparing binding or rate of cleavage at the target sequence between the test and control guiding component reactions. Other assays are possible, and will occur to those skilled in the art.

Surface Properties of the Delivery Platform

The surface properties of the protocell or carrier can be optimized in any useful manner For instance, the lipid bilayer can include appropriate targeting and endosomolytic ligands to promote their cell-specific binding and internalization by various types of immortalized (e.g., Vero, THP-1, A549, and/or HepG2) and primary (e.g., alveolar macrophages and epithelial cells, hepatocytes) host cells, followed by their endosomal escape and cytosolic dispersion within host cells.

Any useful ligand can be employed. The type and density of targeting ligands can be optimized to enhance uptake by the target. Exemplary ligands include a peptide that binds to ephrin B2, which we identified using phage display, to target Vero cells; Fcγ to target THP-1 cells and primary alveolar macrophages; the ‘GE11’ peptide (see, e.g., Li Z et al., FASEB J 2006; 19: 1978-85) to target A549 cells and primary alveolar epithelial cells; the ‘SP94’ peptide (see, e.g., Lo A et al., Molec. Cancer Therap. 2008; 7:579-89) to target HepG2 cells and primary hepatocytes; human complement C3, which binds to receptors on macrophages and dendritic cells; or the ‘H5WYG’ peptide, which ruptures the membranes of acidic intracellular vesicles via the ‘proton sponge’ mechanism (see, e.g., Moore N M et al., J. Gene. Med. 2008 10: 1134-49).

Other ligands include a peptide (e.g., a peptide zip code or a cell penetrating peptide), an endosomolytic peptide, an antibody (including fragments thereof), affibodies, a carbohydrate, an aptamer, a cluster of differentiation (CD) protein, or a self-associated molecular pattern (SAMP) (e.g., as described in Lambris J D et al., Nat. Rev. Microbiol. 2008; 6(2):132; and Poon I K H, Cell Death Differ. 2010; 17:381-97, each of which is incorporated herein by reference in its entirety). Exemplary CD proteins include CD47 OMIM Entry No. 601028, a marker of self that allows RBC to avoid phagocytosis), CD59 (OMIM Entry No. 107271, a marker that prevents lysis by complement), C1 inhibitor (C1INH, OMIM Entry No. 606860, a marker that suppresses activation of the host's complement system), CD200 (OMIM Entry No. 155970, an immunosuppressive factor), CD55 (OMIM Entry No. 125240, a marker that inhibits the complement cascade), CD46 (OMIM Entry No. 120920, a marker that inhibits the complement cascade), and CD31 (OMIM Entry No. 173445, an adhesion regulator and a negative regulator of platelet-collagen interactions). Each recited OMIM Entry is incorporated herein by reference in its entirety.

Any other useful ligand can be employed, such as those identified by the ‘BRASIL’ (Biopanning and Rapid Analysis of Selective Interactive Ligands) method (see, e.g., Giordano R J et al., Nat. Med. 2001; 7:1249-53; Giordano R J et al., Proc. Natl Acad. Sci. USA 2010; 107(11):5112-7; and Kolonin M G et al., Cancer Res. 2006; 66:34-40) to identify novel targeting peptides and single-chain variable fragments (scFvs) via phage display (see, e.g., Giordano R J et al., Chem. Biol. 2005; 12:1075-83; Giordano R J et al., Proc. Natl Acad. Sci. USA 2010; 107(11):5112-7; Kolonin M G et al., Cancer Res. 2006; 66:34-40; Tonelli R R et al., PLoS Negl. Dis. 2010; 4:e864; Lionakis M S et al., Infect. Immun. 2005; 73:7747-58; and Barbu E M et al., PLoS Pathog. 2010; 6:e1000726).

The composition of the lipid layer can include one or more components that facilitate ligand orientation, maximize cellular interaction, provide lipid stability, and/or confer enhanced cellular entry. In one instance, to ensure that targeting ligands are properly oriented on the NanoCRISPR surface, the SLB composition can include DOPC with 30 wt % cholesterol and 5-10 wt % of 1,2-dioleoyl-sn-glycero-3-phosphoethanolamine (DOPE), to which we will conjugate peptides or scFvs with C-terminal cysteine residues using a commercially-available, heterobifunctional amine-to-sulfhydryl crosslinker (SM(PEG)₂₄). The minimum density of targeting ligands necessary can be determined to maximize specific interactions between NanoCRISPRs and model host cells using flow cytometry or surface plasmon resonance to quantify thermodynamic (e.g., dissociation constants) and kinetic (on and off rate constants) binding constants. In another instance, the lipid bilayer includes a phase-separated lipid bilayer.

Biological Packages and Cargos

The delivery platform can include any useful biological package or cargo, including CRISPR components, as well as other cargos (e.g., either associated with the nanoparticle core or the supported lipid bilayer). Biological packages or cargos can include a variety of molecules, including peptides, proteins, aptamers, and antibodies. For instance, combinatorial screens can be performed to identify synergistic effects between CRISPR-based countermeasures or CRISPR components in combination with other agents (e.g., small molecule drugs, such as antimicrobials and/or antivirals).

Exemplary biological packages and/or cargos include an acidic, basic, and hydrophobic drug (e.g., antiviral agents, antibiotic agents, etc.); a protein (e.g., antibodies, carbohydrates, etc.); a nucleic acid (e.g., DNA, RNA, small interfering RNA (siRNA), minicircle DNA (mcDNA) vectors, e.g., that encode small hairpin RNA (shRNA), complementary DNA (cDNA), naked DNA, and plasmid DNA, as well as chimeras, single-stranded forms, duplex forms, and multiplex forms thereof); a diagnostic/contrast agent, like quantum dots, iron oxide nanoparticles, gadolinium, and indium-111; a small molecule; a drug, a pro-drug, a vitamin, an antibody, a protein, a hormone, a growth factor, a cytokine, a steroid, an anticancer agent, a fungicide, an antimicrobial, an antibiotic, etc.; a morphogen; a toxin, e.g., a bacterial protein toxin; a peptide, e.g., an antimicrobial peptide; an antigen; an antibody; a detection agent (e.g., a particle, such as a conductive particle, a microparticle, a nanoparticle, a quantum dot, a latex bead, a colloidal particle, a magnetic particle, a fluorescent particle, etc.; or a dye, such as a fluorescent dye, a luminescent dye, a chemiluminescent dye, a colorimetric dye, a radioactive agent, an electroactive detection agent, etc.); a label (e.g., a quantum dot, a nanoparticle, a microparticle, a barcode, a fluorescent label, a colorimetric label, a radio label (e.g., an RF label or barcode), avidin, biotin, a tag, a dye, a marker, an electroactive label, an electrocatalytic label, and/or an enzyme that can optionally include one or more linking agents and/or one or more dyes); a capture agent (e.g., such as a protein that binds to or detects one or more markers (e.g., an antibody or an enzyme), a globulin protein (e.g., bovine serum albumin), a nanoparticle, a microparticle, a sandwich assay reagent, a catalyst (e.g., that reacts with one or more markers), and/or an enzyme (e.g., that reacts with one or more markers, such as any described herein)); as well as combinations thereof.

Uses

The delivery platform can be employed in any useful manner The present delivery platform can be adapted to recognize the target and, if needed, deliver the one or more cargos to treat that target. Exemplary targets include a cell, a pathogen, an organ (e.g., dermis, vasculature, lymphoid tissue, liver, lung, spleen, kidneys, heart, brain, bone, muscle, etc.), a cellular target (e.g., targets of the subject, such as a human subject, including host tissue, host cytoplasm, host nucleus, etc., in any useful cell, such as e.g., hepatocytes, alveolar epithelial cells, and innate immune cells, etc.); as well as targets for exogenous cells and organisms, such as extracellular and/or intracellular components of a pathogen, e.g., bacteria), a molecular target (e.g., within the subject or the exogenous cell/organism, such as pathogen DNA, host DNA, pathogen RNA, pathogen proteins, surface proteins or carbohydrates of any subject or exogenous cell), etc.

In one instance, the delivery platform is employed to target a host (e.g., a subject), a pathogen, or both (e.g., thereby treating the subject and/or the target). Exemplary pathogens include a bacterium, such as Bacillus (e.g., B. anthracia), Enterobacteriaceae (e.g., Salmonella, Escherichia coli, Yersinia pestis, Klebsiella, and Shigella), Yersinia (e.g., Y. pestis or Y. enterocolitica), Staphylococcus (e.g., S. aureus), Streptococcus, Gonorrheae, Enterococcus (e.g., E. faecalis), Listeria (e.g., L. monocytogenes), Brucella (e.g., B. abortus, B. melitensis, or B. suis), Vibrio (e.g., V. cholerae), Corynebacterium diphtheria, Pseudomonas (e.g., P. pseudomallei or P. aeruginosa), Burkholderia (e.g., B. mallei or B. pseudomallei), Shigella (e.g., S. dysenteriae), Rickettsia (e.g., R. rickettsii, R. prowazekii, or R. typhi), Francisella tularensis, Chlamydia psittaci, Coxiella burnetii, Mycoplasma (e.g., M. mycoides), etc.; mycotoxins, mold spores, or bacterial spores such as Clostridium botulinum and C. perfringens; a virus, including DNA or RNA viruses, such as Adenoviridae (e.g., adenovirus), Arenaviridae (e.g., Machupo virus), Bunyaviridae (e.g., Hantavirus or Rift Valley fever virus), Coronaviridae, Orthomyxoviridae (e.g., influenza viruses), Filoviridae (e.g., Ebola virus and Marburg virus), Flaviviridae (e.g., Japanese encephalitis virus, hepatitis C virus, and Yellow fever virus), Hepadnaviridae (e.g., hepatitis B virus), Herpesviridae (e.g., herpes simplex viruses, herpesvirus, cytomegalovirus, Epstein-Barr virus, or varicella zoster viruses), Papillomaviridae (e.g., papilloma viruses), Papovaviridae (e.g., papilloma viruses), Paramyxoviridae (e.g., respiratory syncytial virus, measles virus, mumps virus, or parainfluenza virus), Parvoviridae, Picornaviridae (e.g., polioviruses and hepatitis A virus), Polyomaviridae, Poxviridae (e.g., variola viruses or vaccinia virus), Reoviridae (e.g., rotaviruses), Retroviridae (e.g., human T cell lymphotropic viruses (HTLV) and human immunodeficiency viruses (HIV)), Rhabdoviridae (e.g., rabies virus), and Togaviridae (e.g., encephalitis viruses, yellow fever virus, and rubella virus)); a protozoon, such as Cryptosporidium parvum, Encephalitozoa, Plasmodium, Toxoplasma gondii, Acanthamoeba, Entamoeba histolytica, Giardia lamblia, Trichomonas vaginalis, Leishmania, or Trypanosoma (e.g., T brucei and T Cruzi); a helminth, such as cestodes (tapeworms), trematodes (flukes), or nematodes (roundworms, e.g., Ascaris lumbricoides, Trichuris trichiura, Necator americanus, or Ancylostoma duodenale); a parasite (e.g., any protozoa or helminths described herein); or a fungus, such as Aspergilli, Candidae, Coccidioides immitis, and Cryptococci. Other pathogens include a multi-drug resistant (MDR) pathogen, such as MDR forms of any pathogen described herein. Additional pathogens are described in Cello J et al., Science 2002; 297:1016-8; Gibson D G et al., Science 2010; 329:52-6; Jackson R J et al., J. Virol. 2001; 75:1205-10; Russell C A et al., Science 2012; 336:1541-7; Tumpey T M et al., Science 2005; 310:77-80; and Weber N D et al., Virology 2014; 454-455c:353-61, each of which is incorporated herein by reference in its entirety.

The delivery platforms of the disclosure can be employed to treat any useful disease that would benefit from genetic knock-out of a known protein. For instance, the platform can be employed to treat a subject from a disease correlated with the presence of that known protein (e.g., a known protein expressed within the subject or within a pathogen infecting that subject). Other diseases include a genetic disorder (e.g., Huntington's disease, hemophilia, sickle cell anemia, metabolic disorders, etc.), in which expression of a known protein is correlated with the disease or its symptoms.

Formulations

The present delivery platform can be formulated in any useful manner For instance, the formulation can be optimized for subcutaneous (SC), intranasal (IN), aerosol, intravenous (IV), intramuscular (IM), intraperitoneal (IP), oral, topical, transdermal, or retro-orbital delivery. Any useful dosages can be employed within the formulations. Exemplary dosages include, e.g., 200 mg/kg.

In particular embodiments, the formulation is optimized for inhalational administration. Inhalational administration of antimicrobial agents has been shown to treat numerous respiratory infections as effectively as IV-injected drugs (see, e.g., Ong H et al., Pharm. Res. 2012; 29:3335-46). Furthermore, formulating nanoparticle-based therapeutics and vaccines as dry powders rather than liquid droplets has been shown to enhance shelf-life in the absence of cold-chain and enable more favorable lung deposition (see, e.g., Kunda N et al., Pharm. Res. 2013; 30:325-41; and Sou T et al., Trends Biotechnol. 2011; 29:191-8). To formulate NanoCRISPRs as inhalable dry powders, the formulation can include spray dried particles with a lung-compatible excipient (e.g., L-leucine). In addition, the aerodynamic diameter, fine-particle fraction, polydispersity index, hygroscopicity, and surface charge of resulting powders can be optimized to maximize deep lung delivery and deposition (see, e.g., McBride A et al., Mol. Pharm. 2013; 10:3574-81; Muttil P et al., Pharm. Res. 2009; 26:2401-16; Muttil P et al., Eur. J. Pharm. Sci. 2007; 32:140-50; Muttil P et al., AAPS J. 2010; 12:330-7; and Muttil P et al., AAPS J. 2010; 12:699-707). Administration can include delivery with an insufflator, which minimizes oropharynx loss. Optionally, Fcγ and the GE11 peptides can be employed to promote uptake of NanoCRISPRs by alveolar macrophages and epithelial cells, respectively.

### EXEMPLARY EMBODIMENTS

Embodiment 1: A carrier comprising a porous nanoparticle loaded with a CRISPR component, wherein the CRISPR component comprises:

i. (a) a guiding component configured to bind to a target sequence or (b) a nucleic acid that encodes a guiding component configured to bind to a target sequence; or

ii. (a) an nuclease or (b) a nucleic acid encoding a nuclease, wherein the nuclease is configured to interact with the target sequence after the guiding component binds to the target sequence.
Embodiment 2: The carrier of embodiment 1, wherein the nanoparticle comprises silica or metal oxide.
Embodiment 3: A carrier comprising:

a biological package, and

a silica shell that encapsulates the biological package.
Embodiment 4: The carrier of embodiment 3, wherein the biological package has a dimension greater than about 20 nm.
Embodiment 5: The carrier of embodiment 3 or 4, wherein the silica shell is porous.
Embodiment 6: The carrier of embodiments 3 or 4, wherein the silica shell is non-porous.
Embodiment 7: The carrier of any one of embodiments 3-6, wherein the silica shell has a thickness of less than about 4 nm.
Embodiment 8: The carrier of any one of embodiments 3-7, wherein the biological package comprises a nucleic acid and/or a polypeptide.
Embodiment 9: The carrier of any one of embodiments 3-8, wherein the biological package comprises a nucleic acid selected from the group consisting of RNA, DNA, and DNA/RNA hybrids.
Embodiment 10: The carrier of any one of embodiments 5-9, wherein the biological package comprises a CRISPR component, wherein the CRISPR component comprises:

i. (a) a guiding component configured to bind to a target sequence or (b) a nucleic acid that encodes a guiding component configured to bind to a target sequence; or

ii. (a) an nuclease or (b) a nucleic acid encoding a nuclease, wherein the nuclease is configured to interact with the target sequence after the guiding component binds to the target sequence.
Embodiment 11: The carrier of any one of embodiments 1, 2, and 10, wherein the CRISPR component comprises:

i. (a) a guiding component configured to bind to a target sequence or (b) a nucleic acid that encodes a guiding component configured to bind to a target sequence; and

ii. (a) an nuclease or (b) a nucleic acid encoding a nuclease, wherein the nuclease is configured to interact with the target sequence after the guiding component binds to the target sequence.
Embodiment 12: The carrier of any one of embodiments 1, 2, 10, and 11, wherein the CRISPR component further comprises a double stranded plasmid DNA.
Embodiment 13: The carrier of embodiment 12, wherein the double stranded plasmid DNA encodes a gene of interest.
Embodiment 14: The carrier of embodiment 12, wherein the double stranded plasmid DNA encodes an siRNA, an shRNA, or an mRNA.
Embodiment 15: The carrier of any one of embodiments 1-14, wherein the guiding component comprises:

a targeting portion comprising a nucleic acid sequence configured to bind to the target sequence; and

an interacting portion comprising a nucleic acid sequence configured to interact with the nuclease.
Embodiment 16: The carrier of any one of embodiments 1-15, wherein the nuclease is a Cas protein or a modified form thereof.
Embodiment 17: The carrier of any one of embodiments 1-16, wherein the carrier further comprises an anticancer agent, an antibacterial agent, or an antiviral agent.
Embodiment 18: The carrier of any one of embodiments 1-17, wherein the carrier further comprises a targeting species that targets a specific cell attached to the surface of the carrier.
Embodiment 19: The carrier of any one of embodiments 1-18, wherein the carrier further comprises a fusogenic peptide attached to the surface of the carrier.
Embodiment 20: A protocell comprising a core surrounded by a lipid bilayer, wherein the core is a carrier according to any one of embodiments 1-19.
Embodiment 21: The protocell of embodiment 20, wherein the lipid layer comprises cholesterol.
Embodiment 22: The protocell of embodiments 20 or 21, wherein the protocell further comprises a targeting species that targets a specific cell attached to the lipid bilayer.
Embodiment 23: The protocell of any one of embodiments 20-22, further comprising a fusogenic peptide attached to the lipid bilayer.
Embodiment 24: A composition comprising a plurality of carriers according to any one of embodiments 1-19, wherein said carriers have a mean diameter of from about 25 nm to about 300 nm.
Embodiment 25: A composition comprising a plurality of protocells according to any one of embodiments 20-23, wherein said protocells have a mean diameter of from about 25 nm to about 300 nm.
Embodiment 26: A composition comprising a plurality of carriers according to any one of embodiments 1-19, wherein said carriers are monodisperse in size distribution.
Embodiment 27: A composition comprising a plurality of protocells according to any one of embodiment 20-23, wherein said protocells are monodisperse in size distribution.
Embodiment 28: The composition according to embodiment 27, wherein the protocells have a mean diameter of from about 25 nm to about 300 nm.
Embodiment 29: A pharmaceutical composition comprising an effective amount of:

a plurality of carriers according to any one of embodiments 1-19;

a plurality of protocells according the any one of embodiments 20-23; or

a composition according to any one of embodiments 24-28;

and a pharmaceutically acceptable carrier, additive or excipient.
Embodiment 30: The pharmaceutical composition of embodiment 29, further comprising a drug which is not disposed within the carrier or protocell.
Embodiment 31: The pharmaceutical composition of embodiment 30, wherein the drug is an anticancer agent, an antiviral agent, or an antibacterial agent.
Embodiment 32: The pharmaceutical composition of any one of embodiments 29-31 in a parenteral dosage form.
Embodiment 33: The pharmaceutical composition of any one of embodiments 29-31 in an oral dosage form.
Embodiment 34: The pharmaceutical composition of any one of embodiments 29-31 in an inhalable dosage form.
Embodiment 35: A method of treating cancer, a bacterial infection, or a viral infection in a patient comprising administering to said patient an effective amount of a pharmaceutical composition of any one of embodiments 29-34 to the patient.
Embodiment 36: A method of treating cancer in a patient comprising administering to a patient an effective amount of a pharmaceutical composition of any one of embodiments 29-34.

The invention is described further in the following non-limiting examples.

### Example 1

Two cell lines were investigated for the delivery of CAS9 and reporter plasmids using mesoporous silica nanoparticles (MSNPs). The reporter plasmid acts as the guide strand, and encodes for GFP. GFP expression services as a CRISPR readout, as the reporter plasmid can only be expressed if CAS9 is also delivered and functioning.

Fluorescence microscopy was used to see GFP expression on human embryonic kidney (HEK 293) and human cervical cancer (HeLa cells, while flow cytometry was used to validate the presence of GFP on HeLas. A control with only nanoparticles was used to establish a gate. LipofectAmine® 2000, a standard transfection agent, was used on both cell lines for comparison of delivery effectiveness.

### Purpose:

To evaluate MSNP/DOTAP complex “protocell” ability to deliver CRISPR plasmid to HeLa cells.

### Methodology:

Load guide strand encoding for the reporter green fluorescent protein gene and CAS 9 complex onto torus-shaped mesoporous silica nanoparticle, 8 nm pore mesoporous silica nanoparticle, and 18 nm pore mesoporous silica nanoparticle. Envelope complex with 1,2-dioleoyl-3-trimethylammonium-propane (DOTAP). After protocell formation, transfect CRISPR plasmids at a 20 μg/mL concentration Image at 48 hrs, perform flow cytometry.

### Procedure:

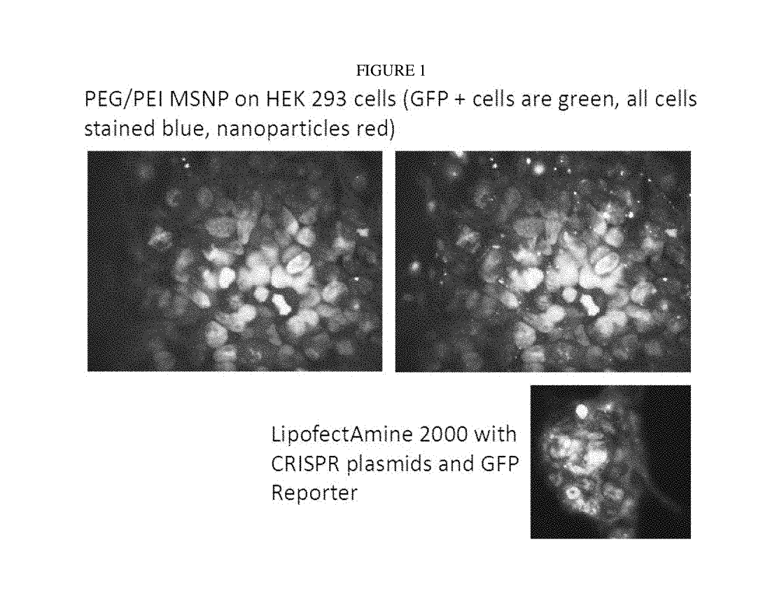

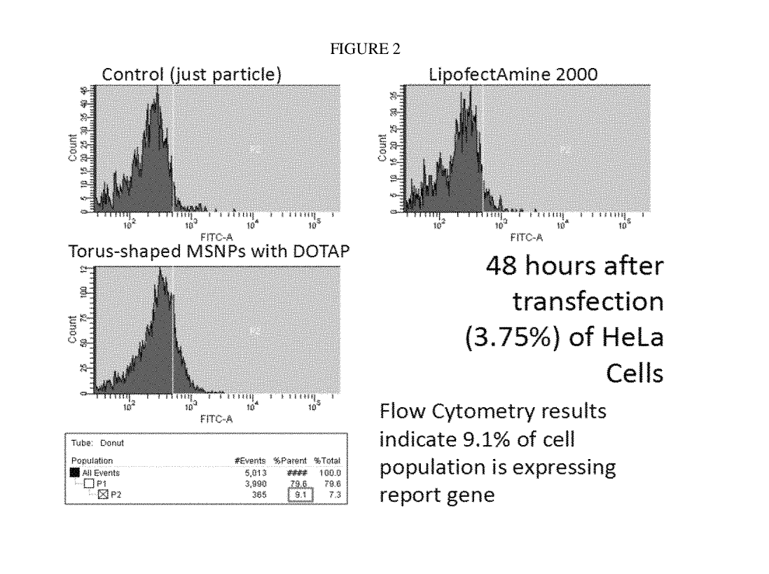

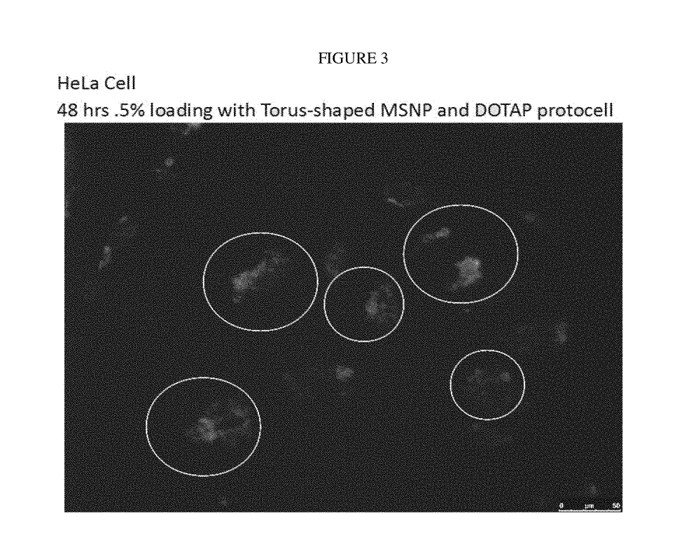

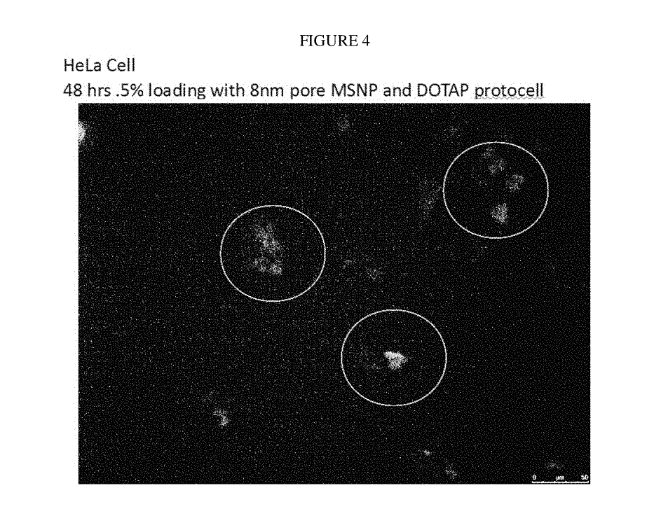

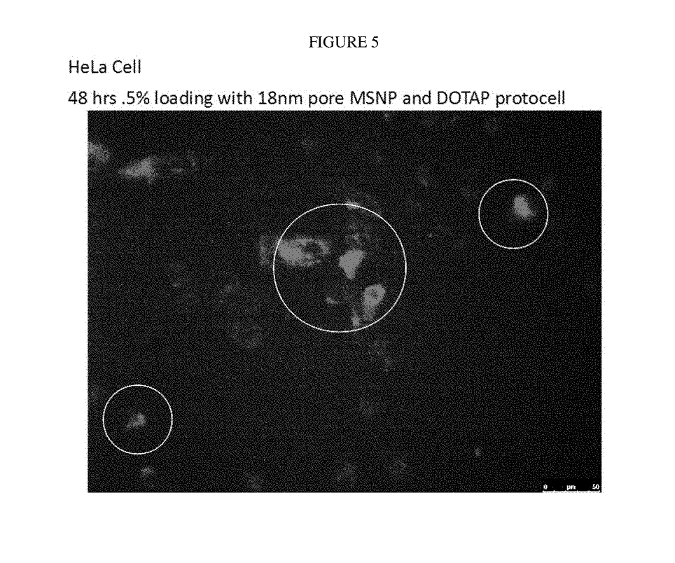

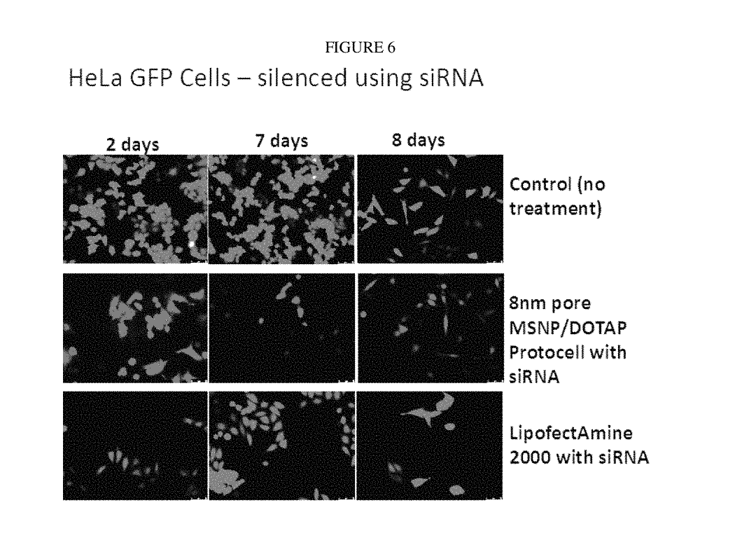

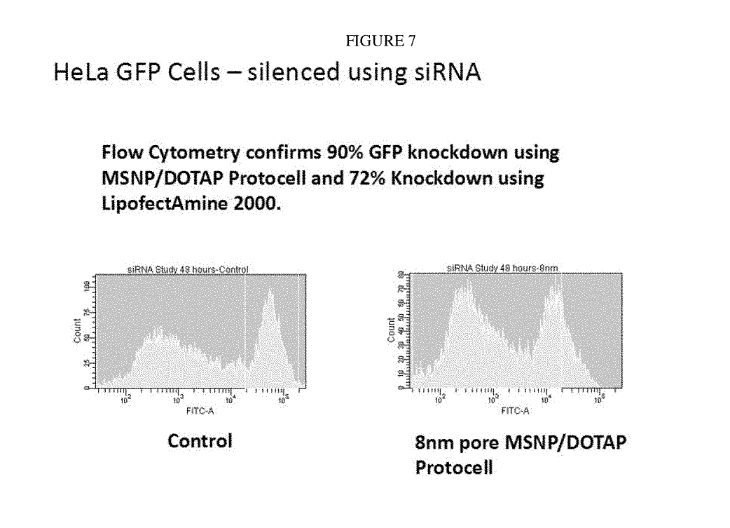

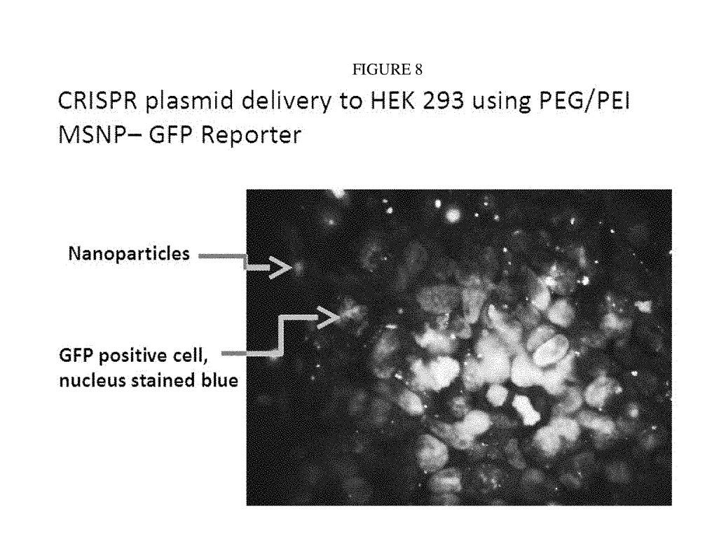

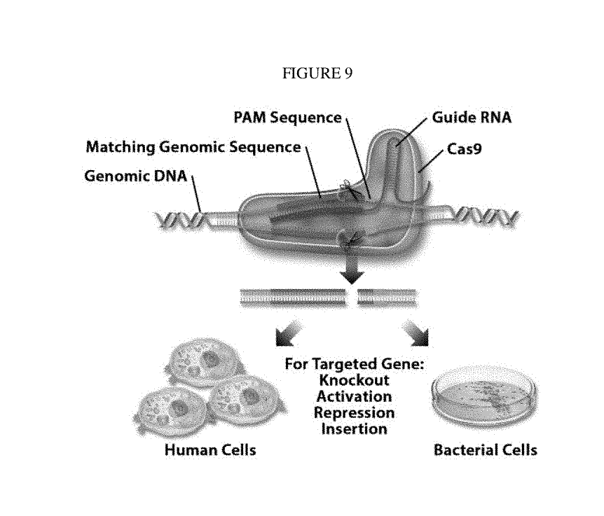

Plate 20000 HeLa cells/well on 48 well plate using 10% FBS in DMEM medium. Incubate at 37° C. and 5% CO₂ for 24 hours.
Prepare DOTAP:
   Vacuum 5 mg of DOTAP
   Rehydrate with 1 mL PBS
   Sonicate until clear (˜1 min.)
   Extrude with 19 mm 0.1 um membranes
Prepare Protocells (for 1 well transfection)
   Mix 0.188 μg of each plasmid together
   Add 5 μg specified nanoparticle
   Incubate 15 min.
   Add 25 μg DOTAP
   Incubate 15 min.
   Spin down @ 20,000 rpm for 10 min.
   Remove supernatant
   Wash with 10 μL PBS
   Add 10 μL PBS, resuspend in sonicator
Transfect cells with 10 μL of prepared solution in well
   The results of experiments performed with protocells according to the present disclosure are set forth in attached FIGS. 1-9.

### Example 2

### Overview of an Exemplary NanoCRISPR Platform

Two urgent problems currently threaten national and global biosecurity: (1) the accelerating emergence of highly virulent, transmissible, drug-resistant pathogens and (2) globally-available, low-cost tools for creating and re-engineering organisms, which greatly increase the odds of accidental or intentional manufacture and release of deadly pathogens. The hallmark of these biothreats is genetic novelty that evolves naturally or is introduced deliberately to enhance virulence and multi-drug resistance, rendering existing countermeasures ineffective.

Treating, managing, and diagnosing nfectious diseases remain a prevailing challenge. To solve this challenge, we herein describe ‘NanoCRISPR,’ a rapid, cost-effective, universal approach to identifying and delivering potent new medical countermeasures against emerging and engineered biological threats. We used CRISPR, a recent, revolutionary discovery having the ability to edit target genes in a highly controlled manner, to develop novel pathogen- and host-directed countermeasures. Then, we packaged CRISPR components within state-of-the-art particle delivery platforms that we have developed. These particle-based delivery platforms provide a flexible platform, in which various particle properties can be modulated to optimize any useful purpose, such as targeted delivery to specific organs, uptake promotion by pathogen-infected cells, and controlled release within appropriate intracellular locations in order to achieve targeted cleavage of pathogen DNA or targeted disruption of pathogen-host interactions. Such properties include size and surface modifications, as well as others described herein. Details are described herein.

### Introduction

There is a need for an approach that rapidly generates medical countermeasures against emerging and engineered pathogens. Emerging and engineered pathogens pose a constant and pressing threat. The natural emergence of highly virulent and transmissible human pathogens is accelerating, due to escalating population densities, increased international trade and travel, and a growing number of human-animal interactions that result from, e.g., wet markets and encroached habitats (see, e.g., Jones K E et al., Nature 2008; 451:990-3). Even when obvious threats like HIV and pandemic influenza are discounted, the twenty-first century has seen several alarming incidents of wide-spread, newly-emerged infectious disease, including the spread of West Nile virus across North America and the 2003 outbreak of SARS in Asia, Europe, Canada, and the U.S., which had an estimated impact on global macroeconomics of $30-100 billion.

Today, ongoing Ebola virus outbreaks in West Africa, SARS-like MERS infections in the U.S., and the first cases of Chikungunya in the western hemisphere are making headlines (see, e.g., Feldmann H, N. Engl. J. Med. 2014; 371:1375-8; Kupferschmidt K, Science 2014; 344:457-8; and Van Bortel W et al., Euro. Surveill. 2014; 19:20759). Similarly, multi-drug resistance (MDR) is becoming frighteningly commonplace, and pathogens that are resistant to all known antimicrobials (e.g., ‘nightmare’ bacteria, like carbapenem-resistant Enterobacteriaceae) are plaguing hospitals and long-term care facilities. These developments have placed a tremendous burden upon the public healthcare system and infrastructure. For example, the CDC estimates that each year in the U.S. alone, more than 2 million people develop serious infections caused by drug-resistant bacteria, with more than 23,000 dying as a result, at a cost of $20 billion in healthcare expenses and $35 billion in lost productivity.

In addition to naturally-emerging pathogens, genetically-enhanced bacteria and viruses pose a serious and eminent threat. Many engineered organisms have dual-use potential (e.g., engineered viruses having genetic modifications that would enable aerosol transmission of the influenza A/H5N1 virus from human-to-human) (see, e.g., Cello J et al., Science 2002; 297:1016-8; Gibson D G et al., Science 2010; 329:52-6; Jackson R J et al., J. Virol. 2001; 75:1205-10; Russell C A et al., Science 2012; 336:1541-7; and Tumpey T M et al., Science 2005; 310:77-80). Thus, there is need for more methodologies and medical countermeasures to reduce the threat posed by naturally-occurring and genetically-altered biological pathogens.

### The CRISPR System

Here, we describe a countermeasure based on the use of the CRISPR system. In 2007, a ground-breaking article described the recently-identified function of a genomic locus present in bacteria and archaea, which the authors termed ‘CRISPR’ (Clustered, Regularly-Interspaced, Short Palindromic Repeats) (see, e.g., Barrangou R et al., Science 2007; 315: 1709-12).

CRISPR functions as an adaptive immune system for prokaryotes to combat foreign genetic sequences introduced by plasmids and bacteriophages (FIG. 10A). Short segments of foreign nucleic acids derived from plasmids or phage are stored in the microbial CRISPR locus and are used to direct sequence-specific cleavage of foreign genetic elements upon subsequent exposure or infection. Different types of CRISPR systems exist, and each system requires a different number of components. For example, Type II CRISPR systems require only three elements: Cas9 (an endonuclease) and two RNA sequences (i.e., trans-activating CRISPR RNA (or tracrRNA) and CRISPR RNA (or crRNA)). The RNA sequence(s) guide Cas9-mediated cleavage of foreign nucleic acids at specific sequences via base complementarity. In another example, Type I CRISPR systems require at least three elements: a Cascade protein complex, a nuclease (Cas3), and one RNA sequence (crRNA). In another example, Type III CRISPR systems generally require at least two elements: one RNA sequence (crRNA, which is usually further processed at the 3′ end) and a Csm or Cmr complex.

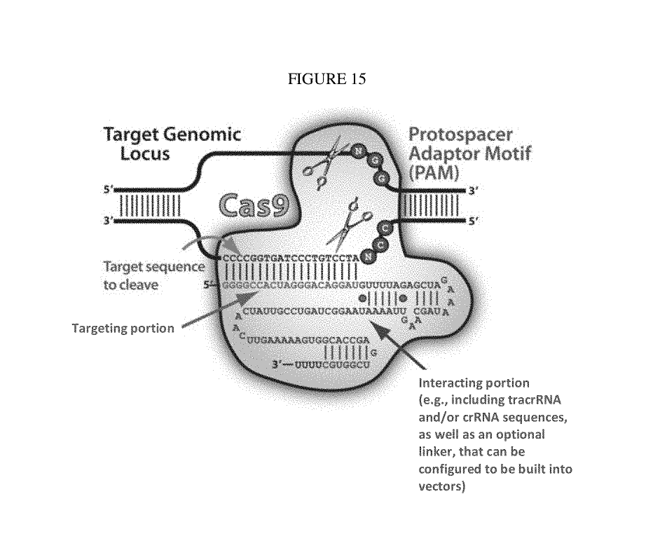

Over the past two years, CRISPR/Cas systems have been used to ‘perform genetic microsurgery’ on mice, rats, bacteria, yeast, plants, and human cells (see, e.g., Mali P et al., Science 2013; 339:823-6; and Zhang F et al., Hum. Mol. Genet. 2014; 23(R1):R40-6). In order to easily manipulate genes using CRISPR, researchers can fuse naturally-occurring tracrRNA and crRNA into a single, synthetic ‘guide RNA’ that directs Cas9 to virtually any desired DNA sequence (see, e.g., FIG. 10B and FIG. 15). The synthetic guide RNA includes at least three different portions: a first portion including the tracrRNA sequence, a second portion including the crRNA sequence, and a third portion including a targeting portion or a genomic specific sequence (gRNA) that binds to a desired genomic target sequence (e.g., genomic target DNA sequence, including a portion or a strand thereof). The chimeric tracrRNA-crRNA sequence facilitates binding and recruitment of the endonuclease (e.g., Cas9), and the sgRNA sequence provides site-specificity to the target nucleic acid, thereby allowing Cas9 to selectively introduce site-specific breaks in the target.

These advances have dramatically increased the rate, efficiency, and flexibility with which prokaryotic and eukaryotic genomes can be altered for purposes ranging from basic research to development of therapeutics to manufacture of biofuels. For biodefense or therapeutic applications, CRISPR technology promises to be the foundation for a nimble, flexible capacity to produce medical countermeasures rapidly in the face of any attack or threat via design of guiding components (e.g., guide RNAs) (this can be accomplished rapidly once the genome of target pathogen has been sequenced) that, upon complexation with a Cas enzyme (e.g., Cas9) and intracellular delivery to an infected host cell, cleave target DNA sequences and inhibit pathogen infection.

While there are a number of other currently-available techniques (e.g., RNA interference) that accomplish the same end, CRISPR-based approaches have longer-lasting effects at lower concentrations and are easier to execute for researchers without extensive training in molecular biology. CRISPR/Cas technologies, therefore, have broad-reaching applications in basic R&D, as well as the manufacture of biofuels with increased productivity and the discovery of novel therapeutics that more effectively treat numerous diseases, including cancer, genetic diseases, infectious disease, autoimmune disorders, and traumatic brain injury.

For biodefense applications, CRISPR can produce medical countermeasures rapidly in the face of any attack or threat' via design of guiding components (something that can be rapidly accomplished once the genome of target pathogen has been sequenced) that, upon complexation with Cas and intracellular delivery to an infected host cell, cleave target DNA sequences and inhibit pathogen infection. Additionally, synthetic CRISPR/Cas systems have sufficient selectivity for target DNA sequences to enable development of both pathogen-directed and/or host-directed countermeasures; this dual-pronged approach promises to kill target pathogens and interrupt critical pathogen-host interactions, thereby dramatically reducing the likelihood that pathogens will evolve resistance. Accordingly, in some embodiments, the present disclosure relates to delivery platforms that can effectively and specifically activate the CRISPR system to immobilize target pathogens.

### Delivery is Critical to Realizing the Potential of CRISPR-Based Medical Countermeasures

In vivo applications of CRISPR require a highly efficacious delivery platform. An example of an ex vivo treatment that forecasts the future of CRISPR-based therapeutics is an HIV adaptive immunotherapy developed by Sangamo BioSciences that is currently in phase II human trials (see, e.g., Manjunath N et al., Viruses 2013; 5(11):2748-66). CRISPR technology is being used in a similar fashion to edit genes responsible for Huntington's disease, hemophilia, sickle cell anemia, and many other devastating genetic disorders. CRISPR also has the potential to cure, not just treat, persistent infections caused by HIV, hepatitis B virus, human papillomavirus, herpes simplex virus, varicella-zoster virus (the causative agents of shingles), and many other viruses that affect millions of people worldwide (see, e.g., Weber N D et al., Virology 2014; 454-455c:353-61). However, in order for the potential of CRISPR-based therapeutics to be realized for these and other diseases, a novel delivery platform capable of encapsulating high concentrations of CRISPR components, delivering them to target organs, tissues, and/or cells in vivo, and releasing them in a controllable fashion, all without causing hypersensitivity or toxicity, must first be developed.

Several viral systems, including adenoviruses, adeno-associated viruses (AAVs), and lentiviruses, have been developed for delivery of nucleic acids and have had some recent successes in the clinic (see, e.g., Mingozzi F et al., Blood 2013; 122(1):23-36). However, these systems have several insurmountable limitations that prevent them from being used to package and deliver CRISPR components. Cas9 expression systems are typically 4-8 kilobase pairs (kbp) in length, making AAV an unsuitable vector, as it is only able to package cassettes <4.2-kbp in length. Additionally, an estimated 70% of the human population has pre-existing anti-AAV antibodies that effectively block AAV-based treatments. In contrast, adenoviruses can accommodate transgenes up to 30-kbp in size; extreme caution is required when using adenoviral vectors, however, as high doses can induce deleterious immune responses, leading to vector toxicity and, in the case of one gene therapy patient, fatality. Lentiviruses present serious safety concerns as well, since they can integrate a significant amount of viral RNA into the host genome and, therefore, have a high oncogenic potential; furthermore, although integrase-deficient lentiviral vectors exist, these vectors are incompatible with DNA cleavage enzymes (see, e.g., Holkers M et al., Nucleic Acids Res. 2013; 41(5):e63).

Non-viral vectors, including liposomes and polymeric nanoparticles, have been developed for delivery of nucleic acids and address the safety concerns posed by viral vectors. These nanoparticle delivery platforms suffer from several limitations, however, including low capacities, uncontrollable release profiles, and complex, specialized synthesis procedures that must be re-adapted for each new cargo molecule, leading to drug- and disease-specific ‘one-off’ approaches (see, e.g., Peer D et al., Nat. Nanotechnol. 2007; 2(12):751-60).

Furthermore, most nanoparticle delivery platforms have highly interdependent properties, whereby changing one property, such as loading efficiency, affects numerous other properties, such as size, charge, and stability. To address these limitations, we propose a flexible, modular platform for highly efficacious delivery of pathogen-directed and host-directed CRISPR components to organs and cells infected with a viral or bacterial pathogen.

Differentiating features of our approach include: (1) employing CRISPR in place of transient genetic knock-down strategies to reliably and controllably ablate expression of target genes; (2) using lipid coated silica (LCS) technologies (e.g., protocells or silica carriers) to develop a safer, more effective CRISPR delivery platform than current, potentially hazardous lentivirus-based vectors; (3) decoupling the challenge of creating an effective therapeutic from the challenge of creating a therapeutic that, itself, has appropriate adsorption, distribution, metabolism, and excretion; (4) employing CRISPR to solve molecular targeting challenges and leveraging features of our LCS technology to solve macroscopic delivery problems; and (5) using an iterative cycle of predictive modeling, simulation, and experimentation to greatly accelerate the design of efficacious NanoCRISPRs. The synergistic combination of these features will allow us to achieve simultaneous delivery of multiple CRISPR constructs that target multiple different genes in pathogens or host cells in order to dramatically reduce the likelihood of the pathogen developing resistance and to rapidly and completely eliminate diverse pathogens.

### NanoCRISPR as a Countermeasure for Pathogens

The NanoCRISPR platform can be adapted as a countermeasure, which can be rapidly prototyped to combat pathogens (e.g., Category A and B pathogens, including smallpox and related orthopoxviruses, hemorrhagic fever viruses, and various bacterial pathogens). The present platform can be designed to focus on the following model pathogens: (1) Rift Valley Fever virus (RVFV), a model RNA virus responsible for several human and livestock epidemics since the 1970s with a hepatic and systemic tropism upon subcutaneous exposure; (2) vaccinia virus (VacV), a model DNA virus that is related to smallpox and has a tropism for the lung upon intranasal exposure and the skin upon intradermal exposure; and (3) Burkholderia pseudomallei (Bp), a model intracellular bacterium that is currently classified as a Category B threat and, upon aerosol exposure, has a tropism for the lung.

The NanoCRISPR platform can be designed to specifically target and effectively inhibit such pathogens. For instance, the platform can be modulated to provide in vivo distribution and delivery that match the tropism of these pathogens. Strategies can be developed to promote targeted uptake of NanoCRISPRs by host cells (e.g., hepatocytes, alveolar epithelial cells, etc.) and for enhancing penetration of CRISPR components into intracellular Bp.

In particular embodiments, the NanoCRISPR platforms allows for the following: (1) use of CRISPR technology in place of transient genetic down-regulation strategies to reliably ablate expression of target genes for controlled periods of time; (2) use of the LCS delivery technology, which has already been demonstrated safe and effective in various animal models, to enable the first in vivo demonstrations of CRISPR-based medical countermeasures; and (3) use of a single NanoCRISPR delivery platform to simultaneously deliver a plurality of CRISPR components that target a plurality of different genes in either the target pathogen or the target host cell, which can greatly improve the probability of eliminating the pathogen, even if individual genes develop natural or man-made resistance.

### Example 3

### Using a Silica Carrier as the NanoCRISPR Delivery Platform

Antimicrobials constitute a first line treatment for bacterial infections. In order to be safe and effective, antimicrobials must (1) be amenable to formulation as an oral tablet, an inhalable solution or powder, or an injectable liquid; (2) be readily absorbed upon administration; (3) accumulate at site(s) of infection while avoiding kidney and liver-mediated clearance; (4) act efficiently and selectively on a molecular mechanism crucial to the viability or virulence of the target pathogen; and (5) be excreted without causing adverse side effects. Many small molecule antimicrobials are effective in vitro but fail in vivo due to low solubility, poor adsorption, high first-pass metabolism, and/or rapid clearance; small molecule antibiotics also ablate normal flora and can have deleterious effects on the host at high doses or upon prolonged exposure.

In contrast, protein and nucleic acid-based antimicrobials can be designed to maximize killing of a target pathogen while minimizing off-target effects on host cells or normal flora; they are far less stable in complex biological fluids (e.g., blood) than small molecule antimicrobials, however, and are typically too large and highly charged to penetrate host and bacterial cell membranes. Therefore, protein and nucleic acid-based countermeasures, especially those that require multiple components, must be packaged within a delivery platform that improves their stability, concentrates them at sites of infection, and promotes their uptake by infected cells or pathogens.

To this end, we propose to couple two powerful technologies: (1) the recently-discovered ‘CRISPR’ technology, which enables rapid modification of specific DNA sequences for tailorable periods of time and (2) an LCS delivery platform, which has high loading capacities physicochemically disparate medical countermeasures, high colloidal stability in blood, tailorable release rates that are triggered by intracellular conditions, high biocompatibility, and tailorable biodistribution and biodegradation. The resulting ‘NanoCRISPR’ technology using a silica carrier (FIG. 10C and FIG. 11C) or a protocell (FIG. 12B and FIG. 13) promises to have the flexibility and scalability needed to rapidly, safely, and completely suppress an outbreak caused by an emerging, drug-resistant, or genetically-enhanced pathogen.

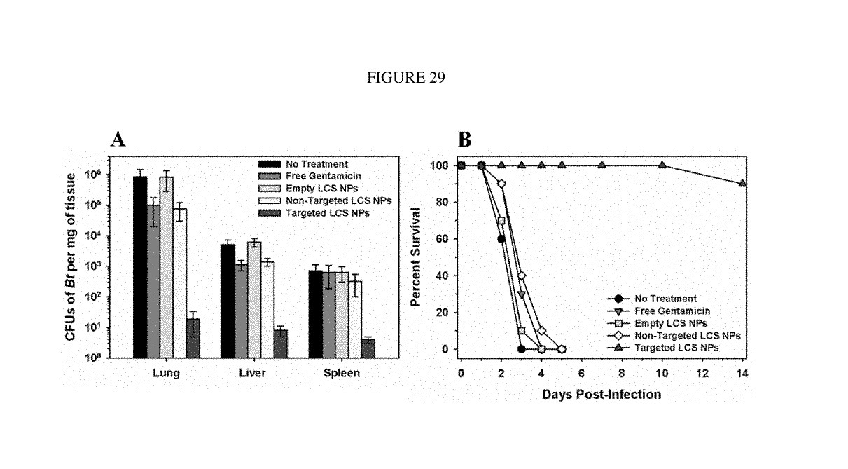

LCS particles possess a unique combination of advantages that enable them to dramatically improve the in vivo efficacy of antibiotics. The synergistic combination of advantages afforded by the silica-based particles and SLB components of LCS particles yields numerous unique features that are not typically possessed by other nanoparticle delivery platforms, including: (1) large loading capacities for physicochemically disparate therapeutics, (2) a high degree of stability in blood, (3) tailorable biodistribution, (4) highly selective internalization by target cells, (5) pH-triggered, intracellular release of encapsulated drugs, (6) tailorable release rates, (7) efficient cytoplasmic dispersion of encapsulated therapeutics, and (8) high biocompatibility and biodegradability. These features allow LCS particles to dramatically improve the in vivo efficacy of gentamicin in BALB/c mice intranasally infected with a fatal dose of gentamicin-resistant Bp (FIG. 29). Furthermore, using a combination of LCS particles that remain in circulation or accumulate in the lung, lymph nodes, spleen, and liver, we can achieve 100% survival when gentamicin-loaded LCS particles are administered up to 96 hours before or 48 hours after infection (FIG. 45), a result that is especially impressive given that all untreated animals became moribund within 72 hours of infection. These data, along with our ability to control biodistribution, to achieve targeted, intracellular delivery, and to mitigate immunogenicity, indicate that we will be able to develop a phage-based therapy that safely and effectively treats acute and chronic respiratory melioidosis

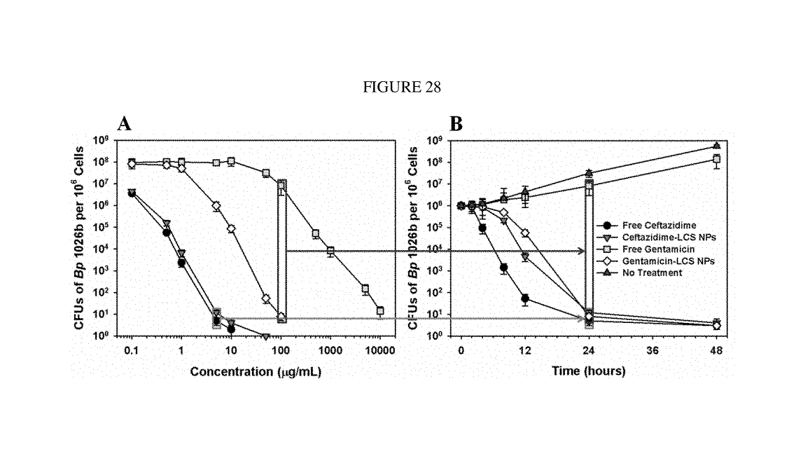

As demonstrated by FIG. 28, LCS particles loaded with gentamicin and targeted to Bp host cells dramatically improve the in vitro efficacy of gentamicin in THP-1 cells infected with Bp 1026b. Since endosomal escape of LCS particles-encapsulated antibiotics is critical to maximize efficacy, the SLBs of LCS particles used in these experiments were further modified with peptides (e.g., R8 in El-Sayed A et al., J. Biol. Chem. 2008; 283(34):23450-61; and HSWYG in Moore N M et al., J. Gene Med. 2008; 10(10):1134-49) that rupture the membranes of acidic intracellular vesicles via the ‘proton sponge’ mechanism.

We have shown that LCS particles, when loaded with gentamicin and targeted to Bp-infected organs, protect 100% of mice from lethal challenge with Bp and dramatically reduce the number of Bp CFUs in relevant organs (2 CFUs in the lungs, 0 CFUs in the liver and spleen), all without causing any signs of toxicity; in comparison, all mice treated with free (i.e., unencapsulated) gentamicin died within 4 days of infection and had overwhelming (10³-10⁵ CFUs/organ) bacterial burdens in their lungs, livers, and spleens. These data, along with our ability to control biodistribution and to achieve targeted, intracellular delivery indicate that we will be able to develop CRISPR-based therapies that safely and effectively treat infections caused by viral (e.g., an Ebola virus) or bacterial (e.g., B. pseudomallei) pathogens.

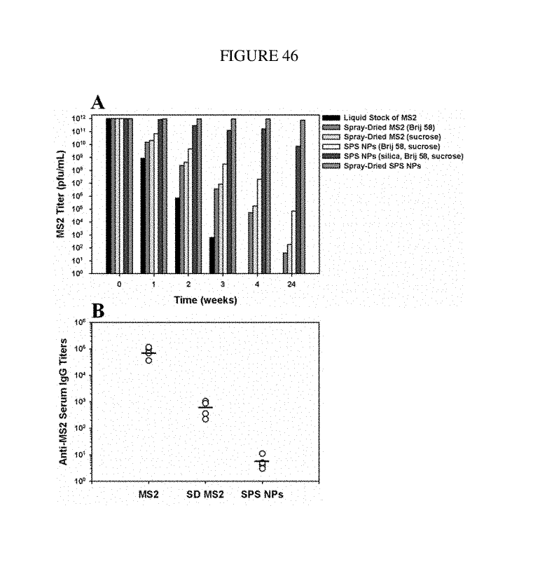

Furthermore, spray-drying of silica carriers stabilized MS2 phage, thereby allowing for long term storage (FIG. 46A) and maintained effectiveness (FIG. 46B). Taken together, silica carriers provide a viable delivery vehicle for any useful biological package (e.g., any described herein).

### Example 4

### Using a Protocell as the NanoCRISPR Delivery Platform

Here, we also describe a NanoCRISPR delivery platform, which couples CRISPR technology with a nanoparticle delivery platform (or a protocell) (FIG. 12B). In order to rapidly generate safer, more effective medical countermeasures, however, the challenges associated with developing therapeutics that target pathogens at the molecular scale must be decoupled from the challenge of creating an effective delivery platform. The NanoCRISPR platform will accomplish this feat by using CRISPR technology to solve molecular targeting challenges and by leveraging features of the protocell technology to solve macroscopic delivery problems.

Using this delivery platform, generic strategies can be developed to accommodate use with any desired target. For instance, such strategies can be employed to rapidly design CRISPR-based countermeasures against emerging or engineered pathogens by creating prototype countermeasures that target model viruses and bacteria and then testing them for in vitro efficacy and biocompatibility. CRISPR components are incorporated into mesoporous silica nanoparticles (MSNPs) and/or encased within a supported lipid bilayer (SLB) that can be modified to promote organ- and cell-specific targeting and release (FIG. 13C).

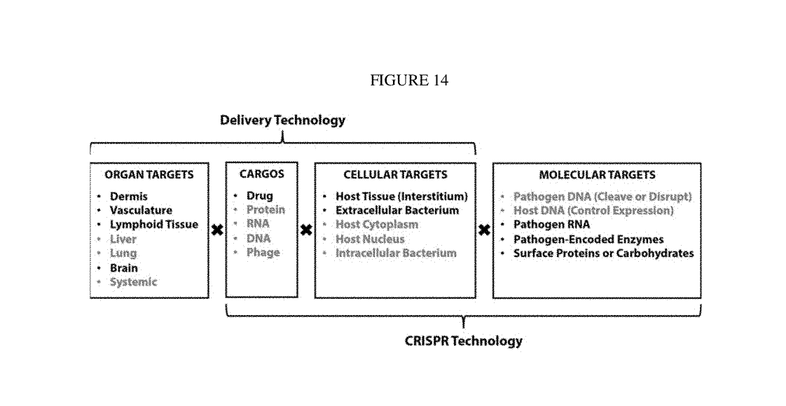

MSNPs encased in SLBs, which we term ‘protocells’, are a revolutionary nanoparticle delivery platform because properties of the MSNP core and SLB shell can be independently modulated to tailor loading and release of physicochemically disparate countermeasures, as well as time-dependent biodistribution and biodegradation. To maximize efficacy of NanoCRISPRs, their biodistributions are engineered based on the tropism of the target pathogen, such as by modifying their surfaces to promote rapid uptake by host cells and tailoring their release rates to facilitate accumulation of CRISPR components in desired intracellular locations. Finally, after optimizing specificity and biodistribution profiles, strategies for facilitating transport of CRISPR components are tested in model bacteria. An exemplary method for implementing this platform strategy is shown in FIG. 14.

### Example 5

### Reproducible and Controlled Production of Protocells and Carriers

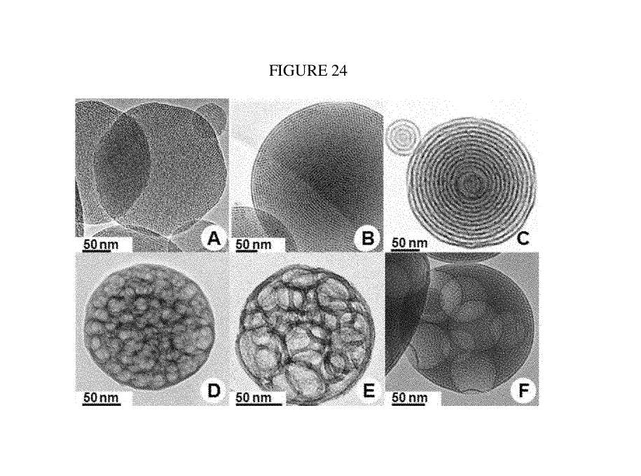

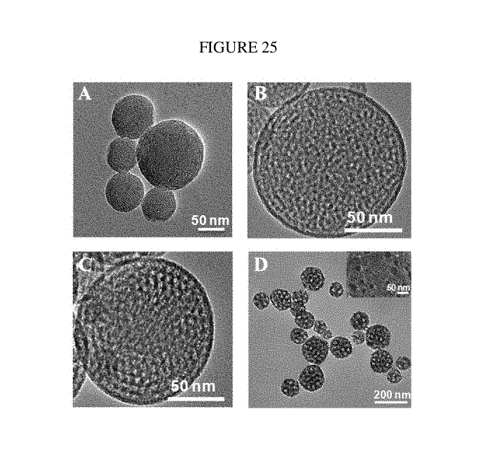

Mesoporous silica nanoparticles (MSNPs) with reproducible properties can be synthesized in a scalable fashion via aerosol-assisted evaporation-induced self-assembly. Aerosol-assisted evaporation-induced self-assembly (EISA)(see, e.g., Lu Y F et al., Nature 1999; 398(6724):223-6 and Brinker C J et al., Adv. Mater. 1999; 11(7):579-85) is a robust, scalable process to synthesize spherical, well-ordered oxide nano- and microparticles with a variety of pore geometries and sizes (FIG. 24 and FIG. 25).

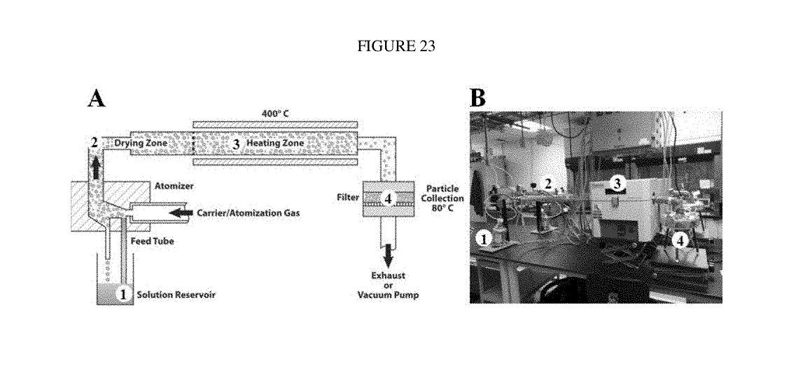

In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor (FIG. 23). Solvent evaporation drives a radially-directed self-assembly process to form particles with systematically variable pores sizes (2 to 50 nm), pore geometries (hexagonal, cubic, lamellar, cellular), and surface areas (100 to >1200 m²/g).

Although spray-drying has been previously used to stabilize phage and adapt them for inhalational administration (see, e.g., Matinkhoo S et al., J. Pharm. Sci. 2011; 100(12):5197-205), aerosol-assisted EISA has several advantages over traditional spray-drying techniques that allow us to precisely control particle size and stability, while maximizing yield and minimizing cost. FIG. 46A shows that carriers (e.g., single phage-in-silica nanoparticles or “SPS NPs”) formed via aerosol-assisted EISA (55 nm mean diameter; one phage per NP, on average) are more stable than spray-dried phage (2.2 μm mean diameter; 42 phage per microparticle, on average).

FIG. 46A also demonstrates the importance of including silica in SPS NP formulations; a model phage (MS2) is ˜16 times more stable upon formulation as SPS NPs that contain silica than upon formulation as SPS NPs that do not contain silica. Furthermore, the silica component of SPS NPs will allow us to precisely control size and release rates, which, in turn, should enable us to tailor biodistribution, maximize phage concentrations at sites of Bp infection, and minimize anti-phage immune responses. As can be seen, SPS NPs dramatically reduced anti-phage antibody responses (FIG. 46B), as compared to liquid stock of MS2 or spray-dried MS2 phage.

Aerosol-assisted EISA, additionally, produces particles compatible with a variety of post-synthesis processing procedures, enabling the hydrodynamic size to be varied from 20 nm to >10 μm and the pore walls to be modified with a wide range of functional moieties that facilitate high capacity loading of physicochemically disparate diagnostic and/or therapeutic molecules. Importantly, aerosol-assisted EISA produces MSNPs that can be easily dispersed in a variety of aqueous and organic solvents without any appreciable aggregation, which enables us to load drugs that have high and low solubility in water.

These particles are also easily encapsulated within anionic, cationic, and zwitterionic supported lipid bilayers (SLBs) via simple liposome fusion. In contrast, particles generated using solution-based techniques aggregate when the pH or ionic strength of their suspension media changes (see, e.g., Liong M et al., J. Mater. Chem. 2009; 19(35):6251-7), typically require complex strategies involving toxic solvents to form SLBs, and have maximum loading capacities of 1-5 wt %, which our MSNPs exceed by an order of magnitude (see, e.g., Cauda V et al., Nano Lett. 2010; 10(7):2484-92; Schloβbauer A et al., Adv. Healthc. Mater. 2012; 1(3):316-20; and Clemens D L et al., Antimicrob. Agents Chemother. 2012; 56(5): 2535-45).

Optimization of pore size and chemistry enables high capacity loading of physicochemically disparate antibiotics, while optimization of silica framework condensation results in tailorable release rates. Despite recent improvements in loading efficiencies and serum stabilities, state-of-the-art liposomes, multilamellar vesicles, and polymeric nanoparticles still suffer from several limitations, including complex processing techniques that are highly sensitive to pH, temperature, ionic strength, presence of organic solvents, lipid or polymer size and composition, and physicochemical properties of the cargo molecule, all of which impact the resulting nanoparticle's size, stability, entrapment efficiency, and release rate (see, e.g., Conley J et al. Antimicrob. Agents Chemother. 1997; 41(6):1288-92; Couvreur P et al., Pharm. Res. 2006; 23(7):1417-50; Morilla M et al., “Intracellular Bacteria and Protozoa” In Intracellular Delivery, ed. A Prokop, pp. 745-811: Springer Netherlands (2011); and Wong J P et al., J. Controlled Release 2003; 92(3):265-73).

In contrast, particles formed via aerosol-assisted EISA have an extremely high surface area (>1200 m²/g), which enables high concentrations of various therapeutic and diagnostic agents to be adsorbed within the pores of the NP by simple immersion in a solution of the cargo(s) of interest. Furthermore, since aerosol-assisted EISA yields particles that are compatible with a range of post-synthesis modifications, the naturally negatively-charged pore walls can be modified with a variety of functional moieties, enabling facile encapsulation of physicochemically disparate molecules, including acidic, basic, and hydrophobic drugs, proteins, small interfering RNA, DNA oligonucleotides, plasmids, and diagnostic/contrast agents like quantum dots, iron oxide nanoparticles, gadolinium, and indium-111.

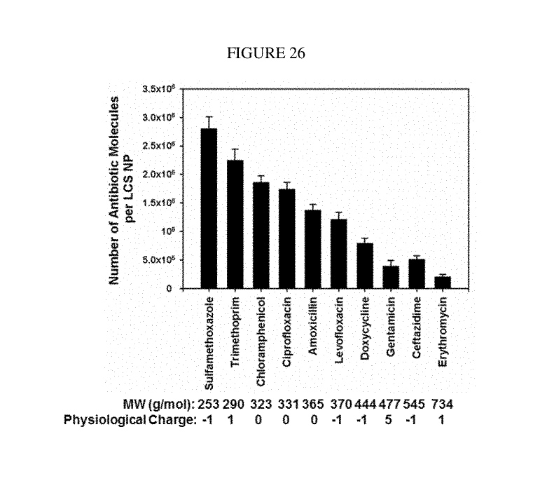

As demonstrated in FIG. 26, particles formed via aerosol-assisted EISA can be loaded with 200,000 to 2,800,000 antibiotic molecules per particle, depending on the molecular weight and net charge of the drug. It is important to note that these capacities are 10-fold higher than other MSNP-based delivery platforms (see, e.g., Clemens D L et al., Antimicrob. Agents Chemother. 2012; 56(5):2535-45) and 100 to 1000-fold higher than similarly-sized liposomes and polymeric nanoparticles (see, e.g., Couvreur P et al., Pharm. Res. 2006; 23(7):1417-50; Morilla M et al., “Intracellular Bacteria and Protozoa” In Intracellular Delivery, ed. A Prokop, pp. 745-811: Springer Netherlands (2011); and Wong J P et al., J. Controlled Release 2003; 92(3):265-73).

It is also important to note that the particles herein (e.g., protocells or carries) can be loaded with complex combinations of physicochemically disparate antibacterials (e.g., three small molecule drugs, an antimicrobial peptide, and a phage), a capability other nanoparticle delivery platforms typically do not possess. We are able to achieve high loading capacities for acidic, basic, and hydrophobic drugs, as well as small molecules and macromolecules by altering the solvent used to dissolve the drug prior to loading and by modulating the pore size and chemistry of the particles. Unlike MSNPs formed using solution-based techniques, particles formed via aerosol-assisted EISA are compatible with all aqueous and organic solvents, which ensures that the maximum concentration of drug loaded within the pore network is essentially equivalent to the drug's maximum solubility in its ideal solvent. Furthermore, since particles formed via aerosol-assisted EISA remain stable upon post-synthesis processing, the pore chemistry can be precisely altered by, e.g., soaking naturally negatively-charged particles in amine-containing silanes (e.g., (3-aminopropyl) triethoxysilane, or APTES), in order to maximize electrostatic interactions between pore walls and cargo molecules.

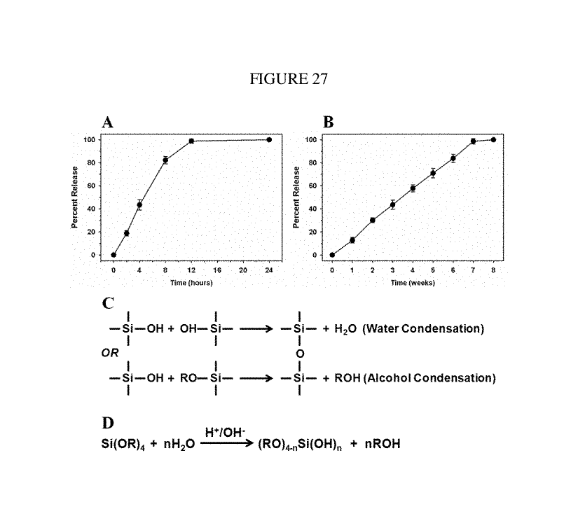

Another unique feature of the delivery platforms herein is that the rate at which encapsulated drugs are released can be precisely modulated by varying the degree of silica framework condensation and, therefore, the rate of its dissolution via hydrolysis under physiological conditions. As shown in FIG. 27, silica (SiO₂) forms via condensation and dissolves via hydrolysis. Therefore, particles with a low degree of silica condensation have fewer Si—O—Si bonds, hydrolyze more rapidly at physiological pH, and release 100% of encapsulated antibiotics within 12 hours.

In contrast, particles with a high degree of silica condensation hydrolyze slowly at physiological pH and can, therefore, release ˜2% of antibiotics (4,000-56,000 antibiotic molecules per particle, based on the loading capacities shown in FIG. 26) per day for 2 months. We can tailor the degree of silica condensation between these extremes by employing different methods to remove structure-directing surfactants from pores (e.g., thermal calcination, which maximizes the number of Si—O—Si bonds vs. extraction via acidified ethanol, which favors the formation of Si—OH bonds over Si—O—Si bonds) and by adding various concentrations of amine or methyl-containing silanes to the precursor solution in order to replace a controllable fraction of Si—O—Si bonds with Si—R—NH₂ or Si—R—CH3 bonds, where R=hydrocarbons of various lengths.

Fusion of liposomes to antibiotic-loaded particles created a coherent SLB that enhances colloidal stability and enables pH-triggered release. Liposomes and multilamellar vesicles have poor intrinsic chemical stability, especially in the presence of serum, which decreases the effective concentration of drug that reaches target cells and increases the potential for systemic toxicity. In contrast, lipid bilayers supported on particles have a high degree of stability in neutral-pH buffers, serum-containing simulated body fluids, and whole blood, regardless of the melting temperature (Tₘ, which controls whether lipids are in a fluid or non-fluid state at physiological temperature) of lipids used to form the SLB.

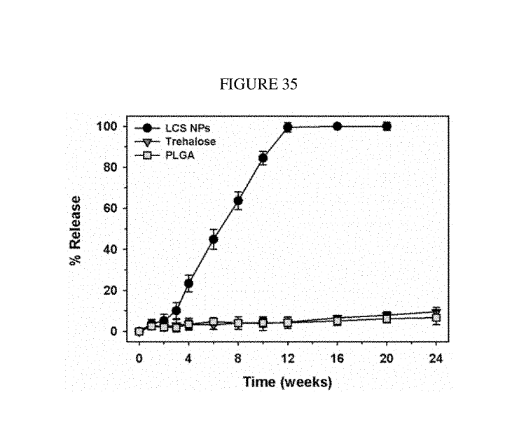

Specifically, we have demonstrated that LCS particles with SLBs composed of the zwitterionic, fluid lipid, 1,2-dioleoyl-sn-glycerol-3-phosphocholine (DOPC) have a high degree of colloidal stability (FIG. 34) in the absence of polyethylene glycol (PEG), which is significant given the FDA's increasing concerns about hypersensitivity reactions induced by PEGylated therapeutics and nanoparticles. LCS particles also have longer room-temperature shelf-lives than liposomes or polymeric nanoparticles, the duration of which can be enhanced by spray-drying them in the presence of excipients that protect the lipid shell from drying and thermal stresses and prevent particle aggregation upon re-suspension (FIG. 35).

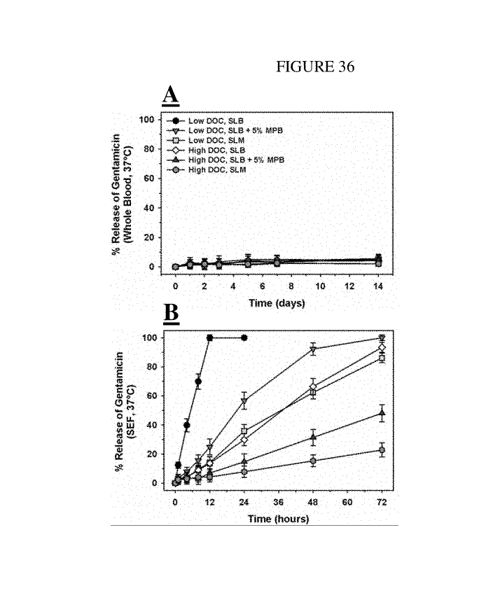

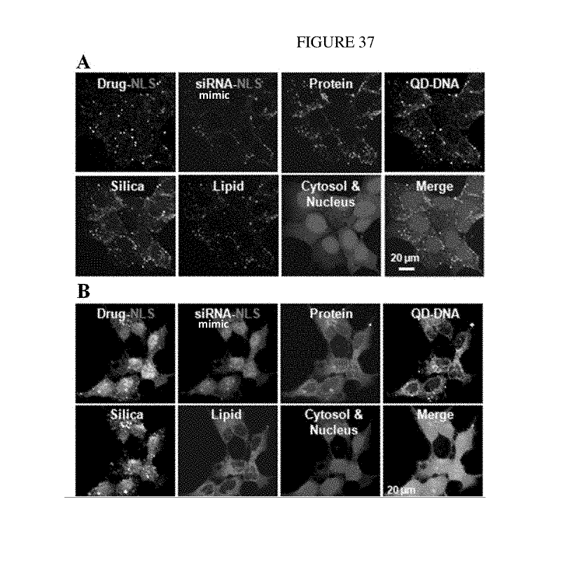

Importantly, LCS particles can be engineered to stably retain encapsulated antibiotics when dispersed in blood (FIG. 36A) but release antibiotics when exposed to conditions that simulate the interior volume of acidic intracellular vesicles, such as endosomes, lysosomes, and phagosomes (FIG. 36B). We have demonstrated that acidic environments destabilize the lipid shell, which exposes the particle core and stimulates its dissolution at a rate dictated by the core's degree of silica condensation. Therefore, by controlling the stability of the lipid shell and the rate at which the particle core dissolves, we can eliminate unwanted leakage of antibiotics in the blood and precisely tailor their intracellular release rates upon uptake of LCS particles by target cells.

### Example 6

### Targeted Delivery Employing the NanoCRISPR Platform

Effective penetration of the NanoCRISPR delivery platform can be promoted in several orthogonal ways. First, the SLB can be optimized with targeting ligands to appropriately bind the target. Second, cell-penetrating peptides can be employed (e.g., associated with the supported lipid bilayer) to facilitate entry. Third, the nanoparticle core can be modified to include a cell penetrating material (e.g., a cell-permeabilizing metal organic framework). Fourth, the LCS delivery platform can be combined with phage technology. All of these strategies can be employed and investigated, in parallel, to provide an effective countermeasure.

Modifying the SLB with targeting ligands promotes efficient uptake of antibiotic-loaded LCS particles by model host cells, which enables efficient killing of intracellular bacteria. In order to inhibit the intracellular replication of bacteria, nanoparticle delivery platforms must be efficiently internalized by host cells, escape intracellular vesicles, and release encapsulated antibacterials in the host cell's cytoplasm. A number of factors govern cellular uptake and processing of nanoparticles, including their size, shape, surface charge, and degree of hydrophobicity (see, e.g., Peer D et al., Nat. Nanotechnol. 2007; 2(12):751-60). Additionally, a variety of molecules, including peptides, proteins, antibodies, and aptamers, can be employed to trigger active uptake by a plethora of target cells.

We have previously shown that incorporation of targeting and endosomolytic peptides that trigger endocytosis and endosomal escape on the LCS particle SLB enables cell-specific delivery and cytoplasmic dispersion of encapsulated cargos. As importantly, we have shown that SLB fluidity can be tuned to enable exquisite (sub-nanomolar) specific affinities for target cells at extremely low targeting ligand densities (˜6 targeting peptides per LCS particle) and that SLB charge can be modulated to reduce non-specific interactions, resulting in LCS particles that are internalized by target cells 1,000 to 10,000-times more efficiently than non-target cells.

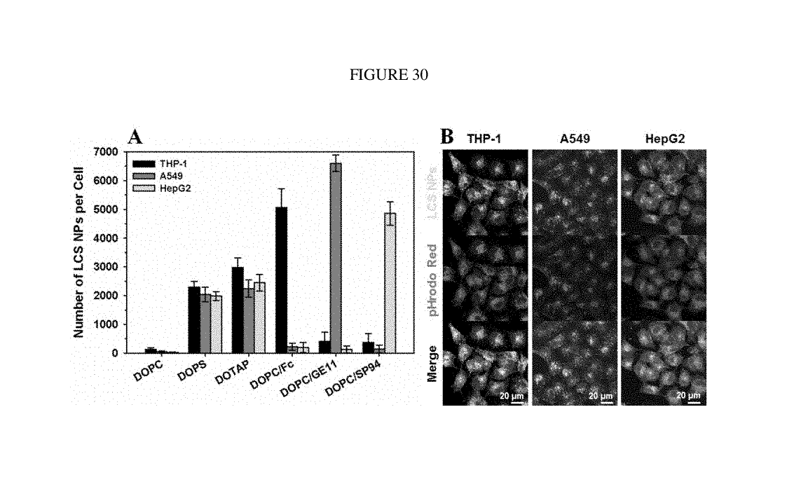

Although originally reported for targeted delivery of chemotherapeutics to cancer, we have utilized the targeting specificity of LCS particles to deliver various antibiotics to host cells in which Bp replicates in vitro. For example, we have shown that modifying DOPC LCS particles with proteins or peptides that target macrophages, alveolar epithelial cells, and hepatocytes triggers a 40 to 200-fold increase in their selective binding and internalization by these cells (FIG. 30). In contrast, LCS particles with SLBs composed of the anionic lipid, 1,2-dioleoyl-sn-glycero-3-phospho-L-serine (DOPS) or the cationic lipid, 1,2-dioleoyl-3-trimethylammonium-propane (DOTAP) were non-specifically internalized by all cell types, which demonstrates an important point: although numerous researchers use cationic lipids and polymers to coat their NP delivery platforms, the resulting non-specific uptake reduces the effective drug concentration that reaches target cells and tissues (see, e.g., Clemens D L et al., Antimicrob. Agents Chemother. 2012; 56(5):2535-45). In some instances, the LCS particles described herein can be employed to encapsulate and deliver physicochemically disparate cargos or agents (e.g., disparate antibacterials, including small molecule antibiotics in combination with peptide, protein, nucleic acid, and/or phage-based bactericidal agents).

### Example 7

### Design of the Silica Carrier Platform

In some instances, the biological packages are sufficiently large (e.g., having a dimension greater than about 20 nm), such that deposition within a pore can be difficult. In one non-limiting instance, phage DNA having more than about 10 kpb can have a compacted dimension of about 40 nm. To accomplish effective delivery of such biological packages, the nucleic acid and/or protein can be delivery by way of a silica carrier, in which a thin shell is deposited around the package. The shell can be formed from biocompatible, biodegradable amorphous silica with or without pores.

Therefore, we will adapt our aerosol-assisted EISA process to coat plasmids and phage with amorphous silica shells of varying thicknesses. To do so, we will combine plasmids (5-5000 ng/mL) or phage (10⁶-10⁹ pfu/mL) with a biocompatible silica precursor solution comprised of a water-soluble silica precursor (e.g., tetraethyl orthosilicate [TEOS]), a biocompatible, USP-grade surfactant (e.g., Pluronic® F68, Pluronic® F127, Brij® 58), a plasmid/phage-stabilizing excipient (e.g., sucrose, mannitol, trehalose, polyvinylpyrrolidone, see, e.g., U.S. Pat. No. 6,077,543; Razavi Rohani S S et al., Int'l J. Pharmaceutics 2014; 465(1-2):464-78); and Vehring R, Pharm. Res. 2008; 25(5):999-1022), and a minute amount of HCl to catalyze condensation of silica precursor molecules into silica (see, e.g., Brinker C J, J. Non-crystall. Solids 1988; 100(1-3):31-50)

We will use a double syringe pump and a small-volume mixer to combine plasmids/phage with the silica precursor solution immediately before they are aerosolized using an ultrasonic spray head, an ultrasonic vibrating nebulizer, or a pressurized aerosol generator; resulting liquid droplets will then be fed into a custom-built, laminar-flow reactor using an inert carrier gas (e.g., N₂, which avoids oxidation of plasmids, phage, and excipients) at the inlet and a weak vacuum at the outlet. Droplets will then pass through multiple heating zones with precisely-controlled temperatures that will drive evaporation-induced self-assembly and condensation of amorphous silica shells around plasmids or phage.

To control biodistribution, uptake by the pathogen, and cytoplasmic release of encapsulated phage, we can modulate various properties of the silica carrier, including hydrodynamic size, surface modification with pH-sensitive lipids and targeting ligands, and route of administration. Any useful formulation may be employed. For instance, since bacterial burden and necrotizing lesions are highest in the lung upon exposure to aerosolized Bp, we will spray-dry SPS NPs with lung-compatible excipients to yield inhalable dry powders; we will then vary the type of excipient and the aerodynamic diameter of the powder to increase phage shelf-life in the absence of cold chain and to maximize alveolar deposition of SPS NPs. Inhalable SPS NPs promise to effectively treat Bp infections that are largely localized in the lung and will likely prove to be efficacious for pre-exposure and urgent post-exposure prophylaxis.

### Example 8

### Design of the Protocell Platform

We have developed scalable strategies for synthesizing highly porous nanomaterials with reproducible properties, thereby providing a way to design the core (e.g., a mesoporous nanoparticle core) of the protocell platform. In this way, the physicochemical properties of the MSNP and SLB can be designed to adapt protocells and related nanoparticle delivery platforms for a wide variety of applications. Here, we describe exemplary design rules to adapt protocells for high capacity loading and controlled release of various countermeasures. We conducted in vitro experiments, and data show that protocells are able to selectively deliver small molecule and nucleic acid-based antivirals to mammalian cells infected with a BSL-2 pseudotype of Nipah virus. Finally, we performed in vivo experiments, which prove that protocells have tailorable biodistributions. These data showed the lack of gross or histopathological toxicity; the presence of ready in vivo degradation and excretion; and the lack of IgG or IgM induction responses, which are indicative of an inflammatory response. These data were observed even when the protocells were modified with high densities of targeting peptides. Additional details follow.

The core of the protocells (e.g., MSNPs) can be prepared with reproducible properties that can be synthesized in a scalable fashion via aerosol-assisted evaporation-induced self-assembly. Aerosol-assisted evaporation-induced self-assembly (EISA) (see, e.g., Lu YF et al., Nature 1999; 398:223-6) is a robust, scalable process that can be employed to synthesize spherical, well-ordered oxide nano- and microparticles with a variety of pore geometries and sizes. In the aerosol-assisted EISA process, a dilute solution of a metal salt or metal alkoxide is dissolved in an alcohol/water solvent along with an amphiphilic structure-directing surfactant or block co-polymer; the resulting solution is then aerosolized with a carrier gas and introduced into a laminar flow reactor. Solvent evaporation drives a radially-directed self-assembly process to form particles with systematically variable pores sizes (e.g., nanopores, such as those having a size of about 2 nm to 50 nm), pore geometries (e.g., hexagonal, cubic, lamellar, etc.), and surface areas (e.g., 100 to >1,200 m²/g).

Aerosol-assisted EISA, additionally, produces particles compatible with a variety of post-synthesis processing procedures, enabling the hydrodynamic size to be varied from 20 nm to more than 10 μm. Further, pore walls can be modified with a wide range of functional moieties that facilitate high capacity loading of physicochemically disparate diagnostic and/or therapeutic molecules.

Various parameters of the core can be optimized in an independent manner For instance, optimization of pore size enabled high capacity loading of physicochemically disparate countermeasures, while optimization of silica framework condensation resulted in tailorable release rates. Despite recent improvements in encapsulation efficiencies and serum stabilities, state-of-the-art liposomes, multilamellar vesicles, and polymeric nanoparticles still suffer from several limitations, including complex processing techniques that are highly sensitive to any number of parameters, e.g., pH, temperature, ionic strength, presence of organic solvents, lipid or polymer size and composition, and physicochemical properties of the cargo molecule. All of these parameters impact the resulting nanoparticle's size, stability, entrapment efficiency, and release rate in a non-straightforward manner (see, e.g., Conley J et al., Antimicrob. Agents Chemother. 1997; 41:1288-92; Couvreur P et al., Pharm. Res. 2006; 23:1417-50; Morilla M et al., “Intracellular Bacteria and Protozoa,” In Intracellular Delivery, ed. A Prokop, 2011, pp. 745-811: Springer, Netherlands; and Wong J P et al., J. Controlled Release 2003; 92:265-73). In contrast, MSNPs formed via aerosol-assisted EISA have an extremely high surface area (e.g., more than about 1200 m²/g), which enables high concentrations of various therapeutic and diagnostic agents to be adsorbed within the core by simple immersion in a solution of the cargo(s) of interest.

In particular, for CRISPR components, MSNPs can be synthesized with pores large enough to accommodate Cas9/gRNA components and/or complexes (e.g., any herein). In addition, the MSNPs can be designed to accommodate any other useful cargo, such as entrapped DNA vectors and, if necessary, cell-permeabilizing metal organic frameworks (MOFs) and Bp phage within MSNPs as they are being formed via aerosol-assisted EISA.

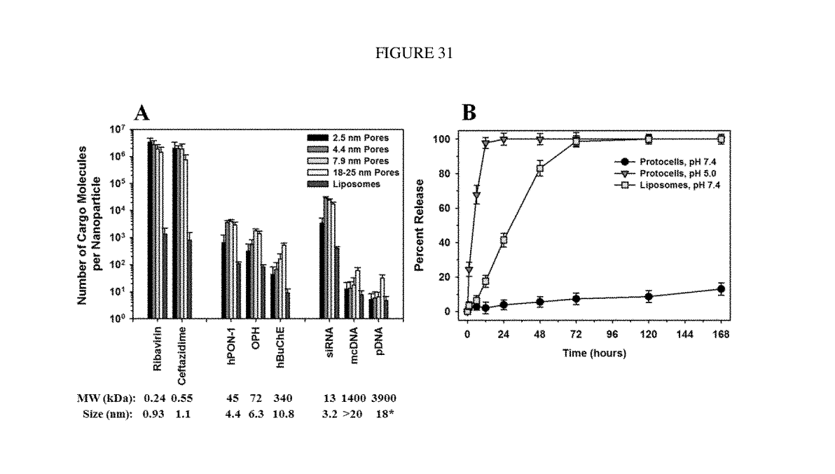

We have previously demonstrated that the loading capacities of MSNPs for various proteins and nucleic acids are maximized when the pore size is slightly larger than the mean hydrodynamic size of the cargo molecule (FIG. 31A). Therefore, in one non-limiting embodiment, MSNPs with pore sizes ranging from 8 nm to 20 nm can be used for encapsulation and delivery of Cas9/gRNA complexes, which have a molecular weight of ˜165 kDa.

Furthermore, since aerosol-assisted EISA yielded MSNPs that are compatible with a range of post-synthesis modifications, the naturally negatively-charged pore walls can be modified with a variety of functional moieties, enabling facile encapsulation of physicochemically disparate molecules, including acidic, basic, and hydrophobic drugs; proteins; small interfering RNA (siRNA); minicircle DNA (mcDNA) vectors that encode small hairpin RNA (shRNA); plasmids (pDNA); and diagnostic/contrast agents like quantum dots, iron oxide nanoparticles, gadolinium, and indium-111 (see, e.g., Ashley C E et al., ACS Nano 2012; 6:2174-88; and Ashley C E et al., Nat. Mater. 2011; 10:389-97).

For instance, NanoCRISPR delivery platforms can include one or more useful surface modifications that promote specific binding and entry of the target. In one instance, NanoCRISPRs can be modified with targeting ligands and endosomolytic ligands to facilitate internalization by model host cells or pathogen cells, as well as endosomal escape and cytosolic dispersion. If needed, BRASIL-based phage display can be employed to identify superior targeting ligands.

As demonstrated by FIG. 31A, MSNPs formed via aerosol-assisted EISA can be loaded with high concentrations of small molecule, protein, and nucleic acid-based countermeasures, and loading capacity is maximized when the pore size is slightly larger than the hydrodynamic size of the cargo molecule. It is important to note that the capacities shown in FIG. 31A are 10-fold higher than other MSNP-based delivery platforms (see, e.g., Clemens D L et al., Antimicrob. Agents Chemother. 2012; 56:2535-45), as well as 100- to 1000-fold higher than similarly-sized liposomes and polymeric nanoparticles (see, e.g., Couvreur P et al., Pharm. Res. 2006; 23:1417-50; Morilla M et al., “Intracellular Bacteria and Protozoa,” In Intracellular Delivery, ed. A Prokop, 2011, pp. 745-811: Springer, Netherlands; and Wong J P et al., J. Controlled Release 2003; 92:265-73). It is also important to note that the MSNPs herein can be loaded with complex combinations of physicochemically disparate countermeasures, a capability other nanoparticle delivery platforms typically do not possess.

Another unique feature of the MSNPs herein is that the rate at which encapsulated drug is released can be precisely modulated by varying the degree of silica framework condensation and, therefore, the rate of its dissolution via hydrolysis under physiological conditions (see, e.g., Ashley C E et al., Nat. Mater. 2011; 10:389-97). The core can be formed from any useful material, such as silica (SiO₂), which forms via condensation and dissolves via hydrolysis. Therefore, MSNPs with a low degree of silica condensation have fewer Si—O—Si bonds, hydrolyze more rapidly at physiological pH, and released 100% of encapsulated drug within 12 hours. In contrast, MSNPs with a high degree of silica condensation hydrolyze slowly at physiological pH and, therefore, released ˜2% of encapsulated drug per day for two months. We can tailor the degree of silica condensation between these extremes by employing different methods to remove structure-directing surfactants from pores (e.g., thermal calcination, which maximizes the number of Si—O—Si bonds versus extraction via acidified ethanol, which favors the formation of Si—OH bonds over Si—O—Si bonds) and by adding various concentrations of amine-containing silanes to the precursor solution in order to replace a controllable fraction of Si—O—Si bonds with Si—R—NH₂ bonds, where R=hydrocarbons of various lengths (e.g., where R is an optionally substituted alkyl, aryl, alkaryl, etc.).

The protocell platform also includes a supported lipid bilayer (SLB). Fusion of liposomes to countermeasure-loaded MSNPs created a coherent SLB that enabled pH-triggered release and provides a biocompatible interface for display of targeting and endosomolytic moieties. Liposomes and multilamellar vesicles have poor intrinsic chemical stability, especially in the presence of serum, which decreases the effective concentration of drug that reaches target cells and increases the potential for systemic toxicity (see, e.g., Couvreur P et al., Pharm. Res. 2006; 23:1417-50; and Morilla M et al., “Intracellular Bacteria and Protozoa,” In Intracellular Delivery, ed. A Prokop, 2011, pp. 745-811: Springer, Netherlands). In contrast, lipid bilayers supported on MSNPs have a high degree of stability in neutral-pH buffers, serum-containing simulated body fluids, and whole blood, regardless of the melting temperature (Tₘ, which controls whether lipids are in a fluid or non-fluid state at physiological temperature) of lipids used to form the SLB (see, e.g., Ashley C E et al., Nat. Mater. 2011; 10:389-97).

Specifically, we have demonstrated that protocells with SLBs composed of the zwitterionic, fluid lipid, 1,2-dioleoyl-sn-glycerol-3-phosphocholine (DOPC) retain small molecule drugs, such as ribavirin, for up to four weeks when incubated in whole blood or a serum-containing simulated body fluid at 37° C. (FIG. 31B). Although protocells are highly stable under neutral pH conditions, the SLB can be selectively destabilized under conditions that simulate the interior volume of intracellular vesicles (e.g., endosomes, lysosomes, and/or macropinosomes), which become acidified via the action of proton pumps. Specifically, DOPC SLBs are destabilized at pH 5.0, which exposed the MSNP core and stimulated its dissolution at a rate dictated by core's degree of silica condensation. Thus, DOPC protocells with MSNPs cores that have a low degree of condensation are, therefore, able to retain ribavirin at pH 7.4 but rapidly release it at pH 5.0 (FIG. 31B).

In order to inhibit the intracellular replication of pathogens, nanoparticle delivery platforms must be efficiently internalized by host cells, escape intracellular vesicles, and release encapsulated countermeasures in the cytosol of host cells. A number of factors govern cellular uptake and processing of nanoparticles, including their size, shape, surface charge, and degree of hydrophobicity (see, e.g., Peer D et al., Nat. Nanotechnol. 2007; 2:751-60).

Additionally, a variety of molecules, including peptides, proteins, aptamers, and antibodies, can be employed to trigger active uptake by a plethora of specific cells. We have previously shown that incorporation of targeting and endosomolytic peptides that trigger endocytosis and endosomal escape on the protocell SLB enables cell-specific delivery and cytosolic dispersion of encapsulated cargos (see, e.g., Ashley C E et al., Nat. Mater. 2011; 10:389-97). As importantly, we have shown that SLB fluidity can be tuned to enable exquisite (sub-nanomolar) specific affinities for target cells at extremely low targeting ligand densities (˜6 targeting peptides per protocell) and that SLB charge can be modulated to reduce non-specific interactions, resulting in protocells that are internalized by target cells 10,000-times more efficiently than non-target cells. Accordingly, the protocell platform can be designed to accommodate and deliver CRISPR component(s) in an effective and targeted manner.

### Example 9

### Biodistribution of the LCS Delivery Platform

For effective in vivo use, any therapeutic agent should be biocompatible. In addition, for targeted therapies, biodistribution should be controlled. Generally, these two characteristics can be difficult to control in an independent manner The platforms herein can be tuned to possess the appropriate biocompatibility and biodistribution based on the associated cargo(s) and/or target (e.g., a subject, such as a human subject; or a pathogen).

Generally, LCS particles are biocompatible, biodegradable, and non-immunogenic. We have evaluated the biocompatibility, biodegradability, and immunogenicity of LCS particles after repeat intraperitoneal (IP) or subcutaneous (SC) injections in Balb/c and C57B1/6 mice. Balb/c mice injected IP with 200 mg/kg doses of DOPC LCS particles three times each week for four weeks showed no signs of gross or histopathological toxicity. Furthermore, we have demonstrated that intact and partially-degraded particles, as well as silicic acid and other byproducts of silica hydrolysis are excreted in the urine and feces of mice at rates that are determined by the dose, route of administration, and biodistribution, observations that are supported by studies performed previously (see, e.g., Lu J et al., Small 2010; 6:1794-805). Finally, we have shown that LCS particles loaded with a therapeutic protein and modified with a high density (˜10 wt % or ˜5000 peptides/LCS particle) of a targeting peptide induced neither IgG nor IgM responses upon SC immunization of C57B1/6 mice at a total dose of 1000 mg/kg.

The biodistribution of LCS particles was controlled by tuning their hydrodynamic size and surface modification with targeting ligands. Since liposomes and multilamellar vesicles are the most similar nanoparticle delivery platforms to LCS particles, the performance of LCS particles were benchmarked against the performance of lipid-based nanoparticles. We found that liposomes and multilamellar vesicles, despite being more elastic that LCS particles, can have biodistribution profiles that are largely governed by their overall size and size distributions, an observation that holds true for LCS particles as well. The sizes of liposomes and multilamellar vesicles are, however, difficult to control and subject to slight variations in lipid content, buffer pH and ionic strength, and chemical properties of cargo molecules (see, e.g., Sommerman E F, “Factors influencing the biodistribution of liposomal systems,” Ph.D. dissertation thesis, Dept. of Biochemistry and Molecular Biology, University of British Columbia, 1986, 163 pp.; Comiskey S J et al., Biochemistry 1990; 29:3626-31; and Moon M H et al., J. Chromatogr. A 1998; 813:91-100). In contrast, the diameter of LCS particles was governed by the size of the MSNP core or, in part, by the thickness of the silica shell, which, as we have described herein, is easy to precisely modulate.

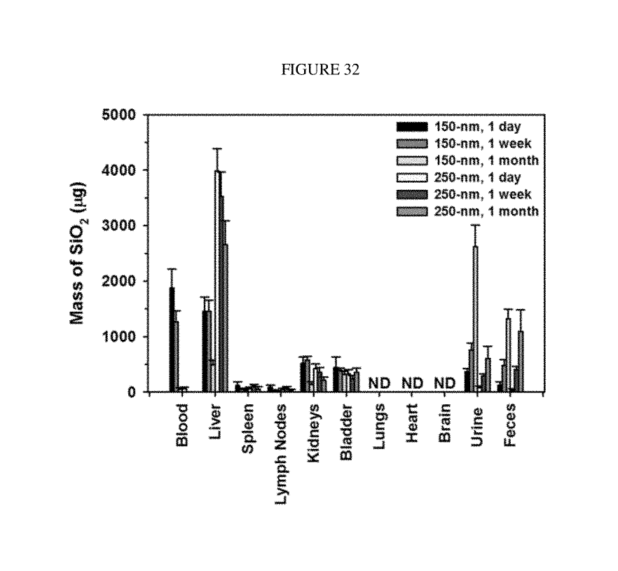

As demonstrated by FIG. 32, the hydrodynamic size of LCS particles dramatically affected their bulk biodistributions: LCS particles (having a diameter of about 250 nm) accumulated in the liver within one hour of injection, while smaller LCS particles (diameter of about 150 nm) remained in circulation for up to two weeks.

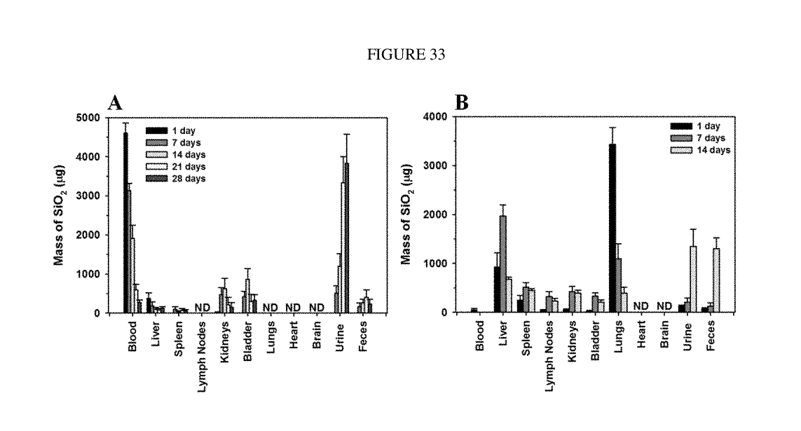

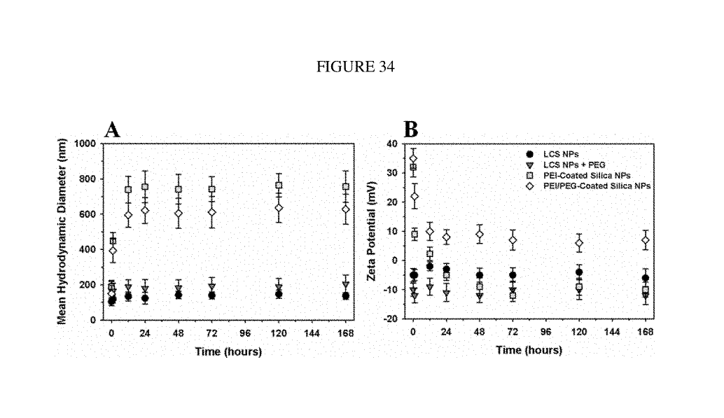

Size-dependent biodistribution can be altered, however, by modifying the surface of DOPC LCS particles with various types of targeting ligands. For example, modifying 150 nm LCS particles with CD47, a molecule expressed by erythrocytes that innate immune cells recognize as ‘self’ (see, e.g., Oldenborg P A et al., Science 2000; 288:2051-4), substantially enhanced their circulation half-life (FIG. 33A). In contrast, modifying 150 nm LCS particles with a proprietary antibody that targets alveolar epithelial cells causes them to rapidly accumulate in the lung (FIG. 33B). Our ability to engineer LCS particles for high capacity, cell-specific delivery of physicochemically disparate medical countermeasures, as well as our ability to achieve both systemic circulation and targeted accumulation within specific organs demonstrates that LCS particles are an excellent platform on which to base NanoCRISPRs.

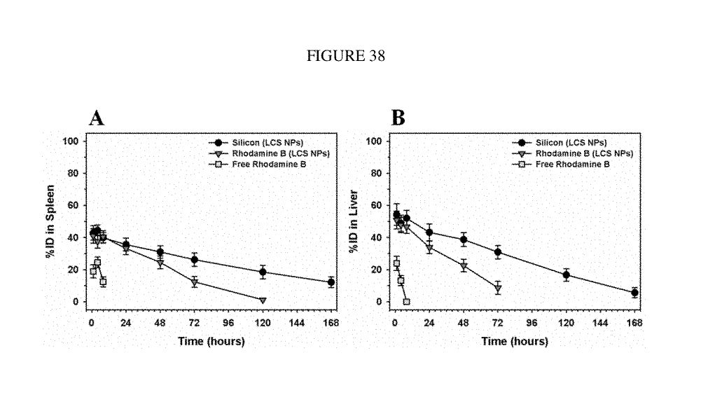

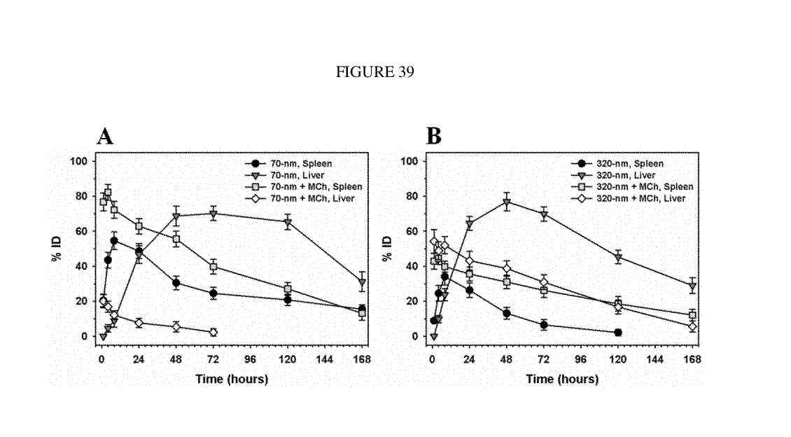

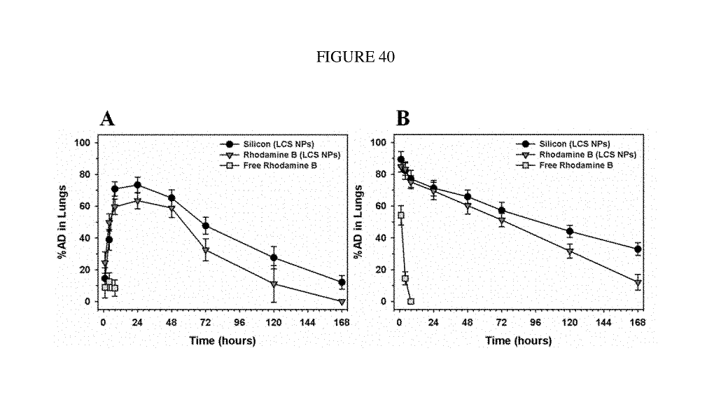

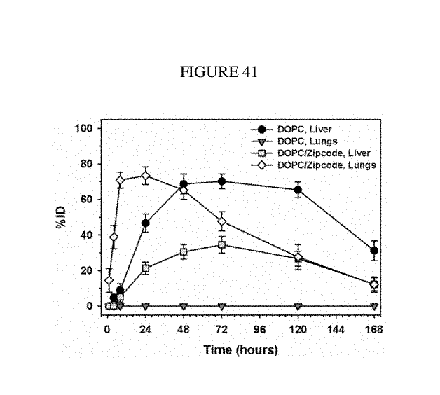

The biodistributions of LCS particles can be controlled by tuning their hydrodynamic diameters, by modifying their surfaces with proteins or peptides that increase circulation times or promote organ-specific accumulation, and by administering them to rodents via parental and non-parental routes. LCS particles that are 320 nm in diameter and modified with Fcγ rapidly accumulate in the lymph nodes, spleen, and liver upon IV injection (FIG. 38 and FIG. 39). LCS particles that are 70 nm in diameter also accumulated in the liver and spleen upon IV injection, but their biodistribution can be shifted to favor the lungs by modifying their surfaces with a peptide ‘zip-code’ that binds to lung vasculature (FIG. 40A and FIG. 41).

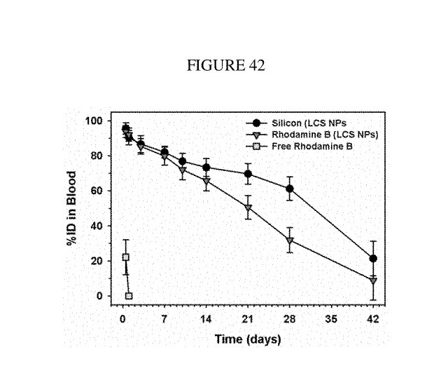

Lung accumulation of LCS particles can also be achieved by delivering them as aerosols; LCS particles that are >100 nm in diameter remain in the lung for up to 7 days (FIG. 40B), while LCS particles that are <100 nm in diameter enter circulation within 8 hours of administration. Finally, LCS particles that are 70 nm in diameter can be engineered to remain in circulation for up to 6 weeks by modifying their surfaces with CD47 (FIG. 42), a protein expressed by erythrocytes that innate immune cells recognize as ‘self’ (see, e.g., Oldenborg P A et al., Science 2000; 288(5473):2051-4). These data demonstrate that LCS particles can be engineered to rapidly accumulate in organs that many viral and bacterial pathogens infect.

Further biodistribution studies can be conducted. A reduced-order model of the circulatory system can be developed based on a network model of the vascular system that includes various organs (e.g., liver, kidneys, lungs) and innate immune cells (e.g., macrophages) (see, e.g., Scianna M et al., J. Theor. Biol. 2013; 333:174-209). For instance multiscale modeling can be employed, as well as ex ovo avian embryo and mouse models, to design, test, and identify NanoCRISPR properties that promote systemic circulation or accumulation in the target organ (e.g., lung, liver, etc.). Exemplary properties include the influence of nanoparticle size, shape, surface charge, surface charge density, and surface modifications on real-time dynamics in the blood. Such modeling can account for dose-dependent biodistribution. If needed, biodistribution can be modified by employing target-specific ligands (e.g., an antibody, a cluster of differentiation (CD) protein, a ligand, a peptide zipcode, etc.) that avoid non-specific interactions, while also avoiding entrapment in the liver and other organs.

### Example 10

### Biocompatibility and Biodegradation of the LCS Delivery Platform

Several reasons support our assertion that the amorphous silica that form the cores or shells of LCS particles have low toxicity profiles in vivo: (1) amorphous (i.e., non-crystalline) silica is accepted as ‘Generally Recognized As Safe’ (GRAS) by the U.S. FDA; (2) recently, solid, dye-doped silica nanoparticles received approval from the FDA for targeted molecular imaging of cancer (see, e.g., He Q et al., Small 2009; 5(23):2722-9; and Chen X et al., Acc. Chem. Res. 2011; 44(10):841); (3) compared to solid silica nanoparticles, MSNPs exhibit reduced toxicity and hemolytic activity since their surface porosity decreases the contact area between surface silanol moieties and cell membranes (see, e.g., Tarn D et al., Acc. Chem. Res. 2013; 46(3):792-801; Zhang H et al., J. Am. Chem. Soc. 2012; 134(38):15790-804; and Zhao Y et al., ACS Nano 2011; 5(2):1366-75); (4) the high internal surface area (>1000 m²/g) and ultra-thinness of the pore walls (<2 nm) enable MSNPs to dissolve, and soluble silica (e.g., silicic acid, Si(OH)₄) has extremely low toxicity (see, e.g., He Q et al., Small 2009; 5(23):2722-9; and Lin Y S et al., J. Am. Chem. Soc. 2010; 132(13):4834-42); and (5) in the case of LCS particles, the SLB further reduces interactions between surface silanol moieties and cell membranes and confers immunological behavior comparable to liposomes.

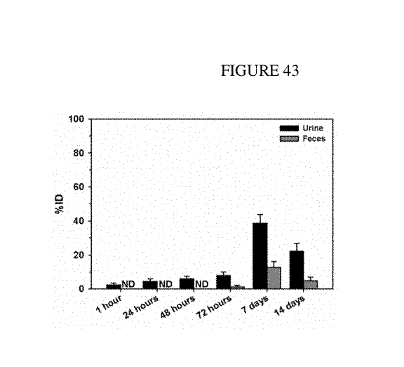

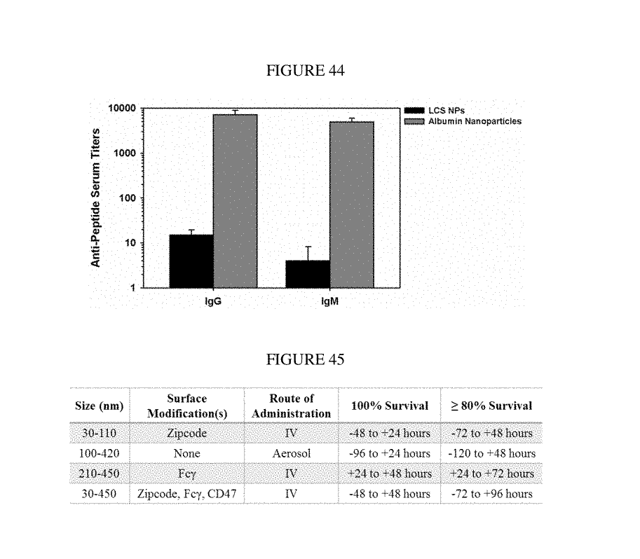

To confirm these observations, we have evaluated the biocompatibility, biodegradability, and immunogenicity of LCS particles after repeat IV or intraperitoneal (IP) injections in mice; BALB/c mice injected IV or IP with large (100 mg/kg) doses of DOPC LCS particles three times each week for 4 weeks showed no signs of gross or histopathological toxicity. Furthermore, we have demonstrated that intact and partially-degraded MSNPs, as well as silicic acid and other byproducts of silica hydrolysis are excreted in the urine and feces of mice at rates that are determined by the dose, route of administration, and biodistribution (FIG. 43). We have shown that LCS particles modified with a high density (˜10 wt % or ˜5000 peptides per particle) of a targeting peptide induce neither IgG nor IgM responses upon SC immunization of C57B1/6 mice at a total dose of 1000 mg/kg (FIG. 44).

### Example 11

### Dual Pathogen Targeting

Any useful pathogens can be targeted using the compositions and methods herein. For instance, one pathogen can be (1) Ebola virus (EBOV), a high viral target; and (2) Burkholderia pseudomallei (Bp), a highly drug-resistant intracellular bacterium. The compositions herein can be configured to target both a virus and a bacterium. To effect this dual targeting approach, the composition can include (1) EBOV-directed countermeasures, comprised of plasmids that encode Cas9 and guide RNAs (gRNAs) that target EBOV RNA within infected host cells; (2) Bp-directed countermeasures, composed of bacteriophages that infect Bp and encode Cas9 and gRNAs that target the Bp genes essential for viability or virulence; and (3) host-directed countermeasures, comprised of plasmids that encode a catalytically inactive variant of Cas9 and gRNAs that temporarily activate or inhibit host genes involved with critical host-pathogen interactions, such as pathogen entry into host cells.

The aerosol-assisted evaporation-induced self-assembly (EISA) process can be employed to encapsulate plasmids and phage within thin layers of mesoporous silica in order to protect them from degradation in the blood, control their rates of intracellular release, and eliminate hypersensitivity reactions associated with intravenous (IV) injection of uncoated plasmids and phage. Then, the silica carriers can further treated (e.g., with targeting ligands) to their surfaces in order to enhance their colloidal stability in blood, reduce their interaction with serum proteins and non-target cells, and promote their accumulation in organs and cells that EBOV or Bp infect.

EBOV primarily infects mononuclear phagocytes, fibroblastic reticular cells, and microvasculature endothelial cells (see, e.g., Sullivan N et al., J. Virol. 2003; 77(18):9733-7), while Bp infects alveolar macrophages and epithelial cells upon respiratory exposure, as well as hepatocytes during later stages of infection (see, e.g., Bast A et al., Front Microbiol. 2012; 10(2):277; and Jones A et al., Infect. Immun. 1996; 64(3):782-90). To enable selective binding, rapid internalization, and cytosolic delivery of NanoCRISPR-encapsulated plasmids and phage, we will use conjugation chemistries to modify the surfaces of lipid and polymer-coated NanoCRISPRs with protein and peptide ligands known to trigger receptor-mediated endocytosis (e.g., E18 peptide (see, e.g., Wu S C et al., Virus Res. 2001; 76(1):59-69), GE11 peptide (see, e.g., Li Z et al., FASEB J. 2005; 19(14):1978-85), SP94 peptide (see, e.g., Lo A et al., Molec. Cancer Therapeut. 2008; 7(3):579-89), mannosylated cholesterol, DEC-205 scFv) and endosomal escape (H5WYG peptide, see, e.g., Moore N et al., J. Gene Med. 2008; 10(10):1134-49) of nanoparticles in cells that are commonly-used to as model EBOV and/or Bp host cells (e.g., Vero, A549, HepG2, THP-1, primary human, monocytes, etc.).

### Example 12

### Design of CRISPR Components

As described herein, the specificity of the CRISPR system depends on the sequence of the guide nucleic acid (e.g., a guide RNA or gRNA). The gRNA can be designed to target a host cell and/or a pathogen cell. For some target, it may be efficacious to target both the host and pathogen cells or, alternatively, only the host or pathogen cells need to be targeted. For instance, the Rift Valley Fever virus (RVFV) is a model RNA virus with a hepatic and systemic tropism, such that host-directed gRNAs for particular lung or epithelial cells may be useful. In another instance, vaccinia virus (VacV, a model DNA virus) and Burkholderia pseudomallei (Bp, a model intracellular bacterium) have particularized tropism (i.e., VacC for the lung, and Bp for the lung), such that host-directed gRNAs for targeting the host's lung cells and the pathogen cells can be combined.

Sequence specificity of guiding components for the target (e.g., pathogen or host) can be determined in any useful manner In addition, additional new target sequences can be identified. For instance, unbiased genome-wide screen can be used to identify novel guiding components that inhibit a pathogen infection by targeting host-pathogen interactions.

CRISPR systems are adaptable immune mechanisms used by many bacteria to protect themselves from foreign nucleic acids introduced by bacteriophages and plasmids (see, e.g., Barrangou R et al., Science 2007; 315:1709-12; and Wiedenheft B et al., Nature 2012; 482:331-8). The Type II CRISPR system from Streptococcus pyogenes has two components: the Cas9 nuclease and guiding component that consists of a crRNA fused to a fixed tracrRNA (FIG. 15) (see, e.g., Mali P et al., Science 2013; 339:823-6). Twenty nucleotides at the 5′ end of the guiding component direct Cas9 to a specific site within target DNA using standard RNA-DNA complementarity. These target sites must be immediately 5′ of a PAM sequence that matches the canonical form, 5′-NGG. Using this system, Cas9 can be directed to cleave any pathogen DNA sequence by designing the first 20 nucleotides of the guiding component to be complementary to the target DNA sequence and contain an adjacent NGG motif.

Since Cas9 is only able to target DNA sequences, the aforementioned approach is unable to directly cleave the genomes of RNA viruses. Catalytically-inactive or ‘dead’ Cas9 (dCas9) bearing mutations that inhibit DNA cleavage can, however, still be recruited by gRNAs to specifically bind target DNA sites (see, e.g., Jinek M et al., Science 2012; 337:816-21). In some instances, dCas9 is a Cas9 catalytic site mutant (e.g., by introducing D10A and H840A mutations to cas9 on pCas9, where Cas9 has the genomic sequence of NCBI Ref. Seq. NC_002737.1 or a protein sequence of UniProtKB/Swiss-Prot: Q99ZW2.1, each sequence being incorporated herein by reference in its entirety). In one instance, Cas9 has a sequence of SEQ ID NO:110, and dCas9 has a sequence of SEQ ID NO:111. Additional Cas protein sequences are provided in SEQ ID NOs:112-117 (FIG. 16A-16H).

Targeting dCas9 to gene promoters has been shown to repress gene expression in both Escherichia coli and human cells (see, e.g., Bikard D et al., Nucleic Acids Res. 2013; 41:7429-37; and Qi L S et al., Cell 2013; 152:1173-83). Additionally, dCas9 fused to a transcriptional activation domain (e.g., VP64 or the p65 subunit of nuclear factor κB) or a transcriptional repression domain (e.g., Krüppel-associated box domain) has been shown to regulate the expression of endogenous genes in human and murine cells (see, e.g., Cheng A W et al., Cell. Res. 2103; 23:1163-71; and Maeder M L et al., Nat. Methods 2013; 10:977-9). Therefore, the present delivery platform can a CRISPR/Cas9 system, as well as adapted or mutated forms thereof (e.g., a CRISPR/dCas9 system), as a host-directed countermeasure that regulates endogenous gene expression in order to disrupt critical pathogen-host interactions or activate host defenses, thereby indirectly inhibiting pathogen infection.

In particular embodiments, NanoCRISPRs can be designed to possess antiviral activity against VacV, a poxvirus that will serve as a model DNA virus. The poxvirus family is comprised of several human pathogens, including monkeypox and smallpox (see, e.g., Cann J A et al., J. Comp. Pathol. 2013; 148:6-21). Due to their high infectivity, their ability to induce devastating disease, the ease with which they can be produced, their high degree of stability, and their potential for aerosolization, smallpox and related poxviruses are classified as Category A priority pathogens. Although smallpox was eradicated through vaccination with VacV, cases of human monkeypox in Africa and vaccinia-like poxvirus infections in South America are on the rise (see, e.g., Damasco C R et al., Virology 2000; 277:439-49; Quixabeira-Santos J C et al.,Emerg. Infect. Dis. 2011; 17:726-9; and Rimoin A W et al., Proc. Natl Acad. Sci. USA 2010; 107:16262-7). For these reasons, we will employ VacV as a model, BSL-2 poxvirus to demonstrate the feasibility of using CRISPR-based strategies to prevent or treat infections caused by virulent poxviruses.

VacV and other poxviruses are large, double-stranded DNA viruses that replicate exclusively in the cytoplasm of infected cells. Cytoplasmic replication requires that these viruses encode RNA and DNA polymerases for viral transcription and genome replication, respectively. We will begin by designing anti-VacV guiding component that target a reporter GFP gene and conserved regions of the viral polymerases. Host-directed guiding component can also be designed to target genes (e.g., Cullin3 ubiquitin ligase, nuclear pore genes, heat shock factor 1, etc.) that have been previously reported to inhibit VacV infection (see, e.g., Filone C M et al., PLoS Pathog. 2014; 10: e1003904; Mercer J et al., Cell Rep. 2012; 2:1036-47; and Sivan G et al., Proc. Natl Acad. Sci. USA 2013; 110:3519-24). Guiding components can be synthesized as oligonucleotides and cloned into a plasmid that encodes both Cas9 and guiding components.

Additional design features, such as placing Cas9 expression under a VacV promoter, can be included to minimize cytotoxicity. In vitro activity of CRISPR-based antivirals can be measured by assessing their influence on GFP expression, cytopathic effect (CPE), and extracellular virus titers. The GFP-producing VacV strain, Western Reserve (WR), is highly cytolytic to cultured cells due to its vigorous replication and virion production; therefore, effective CRISPRs should increase cell viability and decrease both GFP expression and virus titers. We will use similar protocols to those reported in our previous studies (see, e.g., Harmon B et al., J. Virol. 2012; 86:12954-70) to test the dose-response of single, pooled (multiple guiding components that target one gene), and multiplex (single guiding components that target multiple genes; multiple guiding components that target multiple genes) formats in immortalized cell lines (e.g., Vero and A549) and primary alveolar epithelial cells infected with GFP-producing VacV WR. In parallel, we will quantify cell viability using AlamarBlue assays in order to determine selectivity indices, which we define as the ratio of the IC50 value for cell viability to the IC50 value for viral replication. We will use the antiviral cocktail that exhibits the highest selectivity index.

Antiviral NanoCRISPRs can be designed to RVFV, a mosquito-borne, zoonotic, Category A priority pathogen, as a model RNA virus (see, e.g., Hartley D M et al., Emerg. Infect. Dis. 2011; 17:e1; and Mandell R B et al., Hum. Vaccin. 2010; 6:597-601). RVFV infection in humans typically causes an acute febrile illness but can also lead to more severe symptoms, such as retinal vasculitis, encephalitis, and fatal hepatitis with hemorrhagic fever. RVFV is considered a select agent with bioterrorism and agroterrorism potential. There are currently no FDA-approved antivirals for treating infections caused by this pathogen.

We have characterized virus-host interactions for RVFV and have identified multiple host genes whose suppression results in inhibition of RVFV infection. We verified the role of ˜80 genes in RVFV entry and replication using a variety of cellular perturbations, including those induced by small molecule inhibitors, siRNAs, and over-expression of mutant proteins (see, e.g., Harmon B et al., J. Virol. 2012; 86:12954-70). Here, we will target caveolin-1, dynamin-2, and other genes from our genome-wide RNAi screen using dCas9 with and without repressor domains. In addition, we can assess inhibition of GFP-producing RVFV infection using a technique similar to the VacV approach described herein (Vero, HepG2, and primary hepatocytes will be used as model host cells). The CRISPR/dCas9 system can be employed with transcriptional activation domains that target such genes as protein kinase R to inhibit RVFV replication in cells pre-treated with IFN.

NanoCRISPRs can also be designed to possess antimicrobial activity. In particular, Burkholderia pseudomallei can be employed as a model intracellular bacterium. Bp causes the life-threatening disease, melioidosis, in humans and animals and is considered a biodefense threat because of its ability to cause high morbidity and mortality in humans through respiratory inoculation, its broad host range, the ease with which it can be obtained from the environment, its natural resistance to most classes of antibiotics, and the fact that there is currently no approved vaccine. Upon aerosol inoculation, Bp infects many different cell types, including macrophages, neutrophils, and alveolar epithelial cells (see, e.g., Laws TR et al., Microb. Pathog. 2011; 51:471-5; and Harley V S et al., Microbios. 1998; 96:71-93). Once inside host cells, Bp escapes entry vacuoles and replicates within the cytoplasm (see, e.g., Jones A L et al., Infect. Immun. 1996; 64:782-90). Cell-to-cell spreading is accomplished through host cell lysis, as well as actin-based motility (see, e.g., Chan Y Y et al., J. Bacteriol. 2005; 187:4707-19; and Stevens M P et al., Mol. Microbiol. 2005; 56:40-53).

CRISPR-mediated gene disruption in Bp can be tested by targeting genes encoding fluorescent reporter proteins (e.g., GFP). Fully-virulent Bp 1026b can be employed in initial studies, as it is the most thoroughly characterized strain with regard to genome sequence and pathogenesis in cell and animal models of infection (see, e.g., Van Zandt K E et al., Front. Cell Infect. Microbiol. 2012; 2:120). When possible, we will the attenuated Bp strain, Bp82, as it is excluded from Select Agent regulations and can be used under BSL-2 containment (see, e.g., Propst K L et al., Infect. Immun. 2010; 78:3136-43). Bp82 is indistinguishable from 1026b when grown in media replete with adenine and thiamine but is wholly avirulent in multiple animal models of infection (see, e.g., Propst K L et al., Infect. Immun. 2010; 78:3136-43). Reporter genes, as well as the cas9 gene, will be integrated into the genomes of 1026b and Bp82 using standard methods, and gRNAs that target reporter genes will be introduced in RNA or pDNA form via electroporation. Reporter expression will be assessed by fluorescence microscopy and flow cytometry.

Upon establishing a protocol for CRISPR-mediated gene disruption in Bp, we will develop pathogen-directed CRISPR countermeasures by targeting endogenous genes required for viability or virulence (Table 1). Five different guiding components will be used, individually and in combination, to direct disruption of each gene, and the effects will be assessed through enumeration of viable cells (i.e., colony forming units, or CFUs) following transformation (viability) and infection of host cells (virulence).

TABLE 1
Bp genes and their corresponding functions that we can target with guiding
components to effectively kill Bp 1026b in infected host cells. Genes that
are essential for Bp viability are marked with (*), and the remaining genes
are that which are essential for Bp virulence.

| Bp Gene | Function |
|---|---|
| purM, N* | Purine Biosynthesis |
| asd; hisF; ilvI; leuB; trpG; serC* | Amino Acid Biosynthesis |
| pabB* | Folate Biosynthesis |
| aroB, C* | Aromatic Compound Biosynthesis |
| lipB* | Protein Lipoylation |
| mviN | Peptidoglycan Biosynthesis |
| wbi gene cluster (e.g. wbiA) | LPS O-antigen (type II O-PS) |
| | Biosynthesis |
| wcb operon (e.g. wcbB) | Capsule (type I O-PS) Biosynthesis |
| BPSS0417-0429 (e.g. BPS0421) & BPSS1825- | Polysaccharide (type III & IV O-PS) |
| 1832 (e.g. BPSS1833) gene clusters | Biosynthesis |
| type IV pilus operon (e.g. pilA); autotransporter | Adhesin |
| proteins (e.g. bpaC) | |
| fliC | Flagella |
| gspD-N gene cluster (e.g. gspD) | Type II Secretion System |
| bipB-D; bopA, B, E; BSPSS1539 | Type III Secretion System |
| BPSS1496-1511 gene cluster (e.g. BPSS1509) | Type VI Secretion System |
| bpmI3; bpmR3; bpmR5; pmlI; pmlR1 | Quorum Sensing |
| amrAB-oprA operon | Efflux Pump |
| plc-3 | Phospholipase C |

For host-directed CRISPR countermeasures, we will alter mammalian cell host gene expression in ways that promote productive innate immune responses, enabling the prevention or eradication of Bp infection. Treatment with IFNγ or synthetic ligands that activate the pathogen recognition receptor, TLR9, have been shown to potentiate adaptive immune responses that prevent or eradicate Bp infection in vitro and in vivo (see, e.g., Puangpetch A et al., Clin. Vaccine Immunol. 2012; 19:675-83; Tan K S et al., J. Clin. Invest. 2012; 122:2289-300; Easton A et al., J. Infect. Dis. 2011; 204:636-44; and Judy B M et al., PLoS ONE 2012; 7:e34176). Other studies have shown that treatment with rapamycin (induces autophagy), COX-2 inhibitors (down-regulate the prostaglandin E2 signaling pathway), or granulocyte colony stimulating factor (G-CSF, stimulates neutrophil production and activity) can prevent or eradicate Bp infection in vitro and in vivo as well (see, e.g., Cheng A C et al., Clin. Infect. Dis. 2004; 38:32-7; and Cheng A C et al.,Clin. Infect. Dis. 2007; 45:308-14).

To promote innate immune defense, we will use CRISPR to induce expression of the TLR9, IFNG, and CSF3 (G-CSF) genes and repress expression of the PTGS2 (COX-2) gene in the host cell. Constructs that direct production of promoter-targeting guiding component and the dCas9-activator/repressor fusion protein will be introduced into host cells via lentivirus transduction, and their success in promoting productive host defenses will be ascertained through enumeration of Bp CFUs following infection. Individually effective guiding components will be tested in combination, accomplishing simultaneous induction and repression of independent genes through use of orthogonal dCas9 fusion proteins (see, e.g., Esvelt K M et al., Nat. Methods 2013; 10:1116-21).

In addition to altering regulation of pre-identified host defense genes, we can carry out unbiased, genome-wide screens for activating and/or repressing CRISPR constructs that promote defense against infection in vitro. A library of constructs encoding guiding components that target the promoters of all genes in the human genome can be generated through microarray-mediated oligonucleotide synthesis (see, e.g., Wang T et al., Science 2014; 343:80-4; and Shalem O et al., Science 2014; 343:84-7). For instance, five constructs per gene can be synthesized, in which each encoding a guiding component that is designed to independently target the 300 base pairs upstream of the gene's transcriptional start site with minimal off-target effects (see, e.g., Wang T et al., Science 2014; 343:80-4; Shalem O et al., Science 2014; 343:84-7; Fu Y et al., Nat. Biotechnol. 2013; 31:822-6; Hsu P D et al., Nat. Biotechnol. 2013; 31:827-32; Mali P et al., Science 2013; 339:823-6; Qi L S et al., Cell 2013; 152:1173-83; and Nishimasu H et al., Cell 2014; 156:935-49).

The pooled constructs can be incorporated into a vector that directs production of the guiding component, the dCas9-activator/repressor fusion protein, and a fluorescent protein that indicates maintenance of the vector following its introduction into host cells via lentivirus transduction. CRISPR-expressing host cells (THP-1, A549, HepG2) can be infected with our pathogens of interest (e.g., VacV WR, RVFV MP-12, and/or Bp 1026b), and those that survive infection will be recovered for identification of their defense-promoting guiding component constructs via PCR amplification and Next-Generation Sequencing (NGS) (see, e.g., Wang T et al., Science 2014; 343:80-4; and Shalem O et al., Science 2014; 343:84-7). Multiple rounds of screening may be required for sufficient enrichment of survival-conferring guiding component constructs. Top-hit guiding component constructs will be re-tested, pairing them with dCas9-activator/repressor fusion proteins of varying regulatory strength, and using RNA-Seq analysis for simultaneous assessment of both on-target and off-target effects (see, e.g., Qi L S et al., Cell 2013; 152:1173-83; and Gilbert L A et al., Cell 2013; 154:442-51).

CRISPR components can be further modified to facilitate transport into host nuclei or intracellular bacteria. For instance, DNA vectors can be modified with nuclear localization sequences to promote accumulation of CRISPR components in the nuclei of host cells. DNA vectors that encode host- and virus-directed guiding components must be transported into the nuclei of host cells to maximize transcription. We have previously shown that modifying plasmids up to 6000-bp in size with nuclear localization sequences (NLSs) promotes their accumulation within the nuclei of mammalian cells, which enables nearly 100% transfection of dividing and non-dividing cells. Therefore, highly efficient azide-to-alkyne ‘click’ reactions can be employed (see, e.g., Gogoi K et al., Nucleic Acids Res. 2007; 35: e139) to conjugate DNA vectors with peptide-based NLSs derived from any useful protein. Agarose gel electrophoresis will be used to determine the average number of NLS molecules conjugated to each DNA vector; we have previously determined that a 20:1 ratio promotes sufficient accumulation of DNA vectors in mammalian cell nuclei. NLS-modified DNA vectors can then be loaded into nanoparticle delivery platforms as described herein.

Other click-chemistry linkers include the use of one or more chemically co- reactive pairs to provide a spacer that can be transcribed or reverse transcribed. In particular, reactions suitable for chemically co-reactive pairs are preferred candidates for the cyclization process (Kolb et al., Angew. Chem. Int. Ed. 2001; 40:2004-21; and Van der Eycken et al, QSAR Comb. Sci. 2007; 26:1115-326). Exemplary chemically co-reactive pairs are a pair including an optionally substituted alkynyl group and an optionally substituted azido group to form a triazole spacer via a Huisgen 1,3-dipolar cyclo addition reaction; an optionally substituted diene having a 47r electron system (e.g., an optionally substituted 1,3-unsaturated compound, such as optionally substituted 1,3-butadiene, 1-methoxy-3- trimethylsilyloxy-1,3-butadiene, cyclopentadiene, cyclohexadiene, or furan) and an optionally substituted dienophile or an optionally substituted heterodienophile having a 27(electron system (e.g., an optionally substituted alkenyl group or an optionally substituted alkynyl group) to form a cycloalkenyl spacer via a Diels-Alder reaction; a nucleophile (e.g., an optionally substituted amine or an optionally substituted thiol) with a strained heterocyclyl electrophile (e.g., optionally substituted epoxide, aziridine, aziridinium ion, or episulfonium ion) to form a heteroalkyl spacer via a ring opening reaction; a phosphorothioate group with an iodo group, such as in a splinted ligation of an oligonucleotide containing 5′-iodo-dT with a 3 ‘-phosphorothioate oligonucleotide; and an aldehyde group and an amino group, such as a reaction of a 3’-aldehyde-modified oligonucleotide, which can optionally be obtained by oxidizing a commercially available 3′-glyceryl -modified oligonucleotide, with 5 ‘-amino oligonucleotide (i.e., in a reductive amination reaction) or a 5’-hydrazido oligonucleotide.

Exemplary proteins include the SV40 T large antigen (¹²⁶PKKKRKV¹³², SEQ ID NO:11), the heterogeneous nuclear ribonucleoprotein (hnRNP) A1 (²⁶⁸NQSSNFGPMKGGNFGGRSSG PYGGGGQYFAKPRNQGGY³⁰⁵, SEQ ID NO:129), the HIV-1 viral protein, Vpr (⁵²DTWTGVEALIRILQQLLFIHFRIGCRHSRIGIIQQRRTRNGA⁹³, SEQ ID NO:130), and/or the adenoviral Ad3 fiber protein (¹AKRARLSTSFNPVYPYEDES²⁰, SEQ ID NO:131) (see, e.g., Cartier R et al., Gene Therap. 2002; 9:157-67).

Traditional antibiotics (e.g., penicillin) are able to passively diffuse into target bacteria. In contrast, polypeptide and nucleic acid-based antibiotics must be actively introduced into bacteria using one of a variety of transformation techniques, making intracellular delivery of proteins and nucleic acids to pathogenic bacteria within a mammalian host a formidable challenge.

To overcome this challenge, we will investigate three strategies that we believe will enable effective transport of CRISPR components to the cytoplasm of Bp cells in infected cells and animals: (1) modifying guiding component and DNA vectors with cell-penetrating peptides (CPPs) and antimicrobial peptides (AMPs) known to transiently disrupt the cell wall and membranes of Gram-negative bacteria; (2) developing and co-delivering metal organic frameworks (MOFs) that permeabilize Gram-negative bacteria by reacting with phospholipids in the outer and inner membranes; and (3) genetically engineering Bp phage to express Cas9 and guiding components specific for Bp genes that are essential for the bacterium's viability and virulence.

Numerous lytic and non-lytic CPPs and AMPs have been developed that penetrate or permeabilize the membranes of Gram-negative and Gram-positive bacteria via multiple mechanisms (see, e.g., 2010, Handbook of Cell-Penetrating Peptides, Second Edition: CRC Press). To identify the CPP or AMP that maximizes transport of Bp-directed Cas9/guiding component complexes and DNA vectors into Bp cells, we will employ a high-throughput, flow cytometry-based method that has been previously reported in the literature (see, e.g., Benincasa M et al., Antimicrob. Agents Chemother. 2009; 53: 3501-4).

Briefly, CPPs and AMPs that are pre-labeled with the fluorophore, 4,4-difluoro-4-bora-3a,4a-diaza-s-indacene (BODIPY), will be purchased and conjugated in low (10:1), medium (100:1), and high (1000:1) densities to Cas9 via the amine-to-carboxylic acid crosslinker, 1-ethyl-3-[3-dimethylaminopropyl[carbodiimide hydrochloride (EDC), and to DNA vectors using the click chemistry-based technique referenced above. Bp82 cells will then be incubated with various concentrations of BODIPY-labeled conjugates for 1 hour at 37° C., treated with 1 mg/mL of Trypan Blue for 10 minutes at room temperature to quench extracellular BODIPY fluorescence, and analyzed via flow cytometry. The conjugates that result in the highest mean fluorescence intensities will be loaded into nanoparticle delivery platforms (e.g., as described herein) and tested for efficacy in Bp 1026-infected host cells.

It is unknown whether CPPs or AMPs, all of which are relatively short (<35 amino acids, typically), will enhance penetration of macromolecules into bacteria. If Bp-directed CRISPR components conjugated with CPPs or AMPs fail to efficiently penetrate Bp82 or kill Bp 1026b in infected host cells, we will synthesize MOFs that permeabilize Gram-negative bacteria by dephosphorylating phospholipids in the outer and inner membranes. We have previously demonstrated that silica nanoparticles doped with rare earth oxides (e.g., lanthanum) permeabilize bacteria by reacting with phospholipids in the cell membrane(s). Since resulting phosphate salts (e.g., lanthanum phosphate) are toxic to mammalian cells, however, we will develop MOFs that have the same mechanism of action but have dramatically increased biocompatibility. MOFs are crystalline, nanostructured materials composed of metal ions joined to organic ‘linker’ groups (e.g., optionally substituted bivalent alkyl, alkaryl, or aryl groups, as described herein). MOFs have an unprecedented degree of synthetic flexibility, which should enable us to synthesize a MOF or cocktail of MOFs that can permeabilize bacteria without impacting the viability of host cells.

MOFs also have exquisite thermal and chemical robustness, which can be beneficial for successful integration within nanoparticle delivery platforms. Recent theory and experiments have demonstrated that UiO-, MIL- and pyra-MOFs have high affinities towards phosphate groups (see, e.g., Barea E et al., Chem. Soc. Rev. 2014; 43:5419; Katz M J et al., Angew. Chem. Int. Ed. 2014; 53:497-501; and Montoro C et al., J. Am Chem. Soc. 2011; 133:11888-91). We will expand upon this work to generate MOFs with metal centers and/or functional groups (e.g., —OH, —NH₂, —SO₃H, etc.) that irreversibly bind phosphate moieties in phospholipids without releasing cytotoxic byproducts. We will then optimize the synthesis procedure for each MOF to yield nanoparticles ≦30 nm in diameter; minimizing the size of bacteria-permeabilizing MOFs will be critical to efficiently encapsulate them within nanoparticle delivery platforms. We will use the flow cytometry technique described above to assess penetration of fixed concentrations of BODIPY-labeled, Bp-directed Cas9/guiding component complexes and DNA vectors into Bp82 in the presence of increasing concentrations of MOFs. If MOFs enhance penetration of CRISPR components at concentrations that can reasonably be loaded into nanoparticle delivery platforms, we will test their in vitro efficacy.

In addition, target permeability can be optimized by employing genetically engineered phage, which has a broad host range for Bp (see, e.g., Gatedee J et al., Virology J. 2011; 8:366; Kvitko B et al., BMC Microbiol. 2012; 12:289; and Yordpratum U et al., FEMS Microbiol. Lett. 2011; 314:81-8). Such phages can be used to express Cas9 and a battery of guiding components that target genes essential for Bp viability and virulence (Table 1) (see, e.g., Propst K L et al., Infect. Immun. 2010; 78:3136-43; Atkins T et al., Infect. Immun. 2002; 70:5290-4; Breitbach K et al., Trans. R. Soc. Trop. Med. Hyg. 2008; 102:589-94; Pilatz S et al., Infect. Immun. 2006; 74:3576-86; Silva E B et al., Infect. Immun. 2013; 81:4626-34; Rungpanich U et al., Southeast Asia J. Trop. Med. Public Health 2009; 40:295; Rodrigues F et al., J. Bacteriol. 2006; 188:8178-88; Haque A et al., J. Infect. Dis. 2006; 194:1241-8; Cuccui J et al., Infect. Immun. 2007; 75:1186-95; Norris M H et al., Infect. Immun. 2011; 79:4010-8; Easton A et al., J. Infect. Dis. 2011; 204:636-44; Foulongne V et al., Infect. Immun. 2001; 69:547-50; Ling J M L et al., Canadian J. Microbiol. 2006; 52:831-42; DeShazer D et al., Molec. Microbiol. 1998; 30:1081-100; Wikraiphat C et al., FEMS Immunol. Med. Microbiol. 2009; 56:253-9; Reckseidler S L et al., Infect. Immun. 2001; 69:34-44; Atkins T et al., J. Med. Microbiol. 2002; 51:539-53; Reckseidler-Zenteno S L et al., Infect. Immun. 2005; 73:1106-15; Warawa J M et al., Infect. Immun. 2009; 77:5252-61; Sarkar-Tyson M et al., J. Med. Microbiol. 2007; 56:1005-10; Chua K L et al., Infect. Immun. 2003; 71:1622-9; DeShazer D et al., J. Bacteriol. 1999; 181:4661-4; Stevens M P et al., Molec. Microbiol. 2002; 46:649-59; Stevens M P et al., J. Bacteriol. 2003; 185:4992-6; Ulrich R L et al., Infect. Immun. 2004; 72:1150-4; Stevens M P et al., Microbiol. 2004; 150:2669-76; Suparak S et al., J. Bacteriol. 2005; 187:6556-60; Warawa J et al., FEMS Microbiol. Lett. 2005; 242:101-8; Burtnick M N et al., Infect. Immun. 2008; 76:2991-3000; Burtnick M N et al., Infect. Immun. 2011; 79:1512-25; Ulrich R L et al., J. Med. Microbiol. 2004; 53:1053-64; Valade E et al., J. Bacteriol. 2004; 186:2288-94; Jeddeloh J A et al., Infect. Immun. 2003; 71:584-7; Tuanyok A et al., Infect. Immun. 2006; 74: 5465-76; and Boddey J A et al., Cell. Microbiol. 2007; 9:514-31).

CRISPR systems occur naturally in phage (see, e.g., Seed K D et al., Nature 2013; 494:489-91), and their incorporation into phage can be accomplished using CRISPR itself (see, e.g., Kiro R et al., RNA Biology 2014; 11:42-4) or conventional methods for phage genome editing. Furthermore, phage performance can be optimized using a synthetic biology approach, where expression of Bp-directed guiding component will be placed under the control of a synthetic regulatory circuit engineered into the phage genome. This circuit will feature sequences that encode a transcriptional repressor protein and a Cre-Lox recombination system. The repressor will recognize a high-affinity binding site, which will be inserted within the promoter for the guiding component construct, as well as a slightly lower affinity binding site, which will be inserted in the promoter for the cre gene; binding sites of varying strength will be generated using a screening strategy based on SELEX and NGS (see, e.g., Jolma A et al., Genome Res. 2010; 20:861-73). Due to the difference in binding site affinity, the repressor will wholly prevent expression of guiding component but allow low-level expression of Cre.

Daughter phage can be produced until Cre levels are sufficient to catalyze recombination at the Lox sites, which will delete the repressor gene from the phage genome, halt expression of the repressor, and release the promoter that drives guiding component expression. At this point, Cas9 will be present at high levels due to constitutive expression, so production of guiding component(s) will trigger rapid disruption of the genes that they target, which will, in turn, cause Bp cells to lyse and release daughter phage for another round of infection.

If needed, we can also design and include key regulatory circuit using synthetic biology principles (see, e.g., Randall, A et al., “Design and Connection of Robust Genetic Circuits,” In Methods in Enzymology, pp. 159-8, 2011; Marchisio M A, Methods Mol. Biol. 2012; 813:3-21; and Riccione K A et al., ACS Synth. Biol. 2012; 1:389-402) and genetic components from Bp and/or the BioBricks repository (see, e.g., Smolke C D, Nat. Biotechnol. 2009; 27:1099-102; Sleight SC et al., ACS Synth. Biol. 2013; 2:506-18; and Rokke G et al., Methods Mol. Biol. 2014; 1116:1-24). Genetic circuits and engineered phage will be tested using cultures of Bp82. If engineered phage lyse Bp82, then the selected phage can be encapsulated within nanoparticle delivery platforms.

### Example 13

### Design Principles for Viral Targets

Type II CRISPR systems from such bacteria as Streptococcus pyogenes (Sp) and Francisella novicida (Fn) are comprised of a Cas9 endonuclease and a guiding component, where 20 nucleotides at the 5′ end of the guiding component direct Cas9 to a specific site within a target DNA sequence using RNA-DNA complementarity; targets sites must be immediately 5′ of a DNA sequence, known as the ‘protospacer adjacent motif’ (PAM), with the canonical form, 5′-NGG. Since EBOV has a single-stranded, negative-sense RNA genome, and Cas9/guiding component systems cleave DNA targets, we will explore two options for using CRISPR/Cas systems to directly inhibit EBOV infection: (1) use the SpCas9/guiding component system in combination with a PAM-presenting oligonucleotide (PAMmer), which was recently shown to stimulate site-specific endonucleolytic cleavage of single-stranded RNA targets, in order to cleave EBOV RNA (see, e.g., O'Connell M R et al., Nature 2014; 516(7530):263-66); and (2) use the SpCas9/guiding component or FnCas9/guiding component system to bind to and inactivate EBOV RNA, an approach that was recently shown to inhibit hepatitis C replication in vitro (see, e.g., Price A A et al., Proc. Natl Acad. Sci. USA 2015; 112(19):6164-9).

To design guiding components that target EBOV RNA for cleavage or inactivation, we will analyze the genomes of multiple EBOV strains, including Zaire (the strain responsible for the 2014 West African outbreak), Sudan, and a mouse-adapted strain, to identify regions that are highly conserved and are, therefore, good targets for broad-spectrum CRISPR countermeasures against EBOV. Bioinformatic programs can be employed to design 10 guiding components that target multiple EBOV strains but have little to no complementarity to host (e.g., human, mouse, etc.) genomes. We will synthesize SpCas9/guiding component or FnCas9/guiding component expression vectors for each guiding component sequence where each plasmid encodes a guiding component and either active Cas9 and a PAMmer olignonucleotide or an inactive Cas9; inactive forms of SpCas9 or FnCas9 will be used when the Cas9/guiding component system inactivates but does not cleave EBOV RNA in order to increase safety. In one non-limiting instance, EBOV-directed plasmids can lack nuclear localization sequences since EBOV replicates in the cytosol.

To ensure optimal binding of the Cas enzyme, a synthetically evolved Cas9 variant can be employed that efficiently and specifically cleaves single-stranded RNA molecules in order to increase the efficacy and safety of EBOV-directed CRISPR countermeasures. For instance, directed evolution (e.g., including rational design, random mutation, addition or deletion of amino acids, methylation-based selection, combination of recognition and cleavage domains from different enzymes, combinatorial screening methods, see, e.g., Dorr B M et al., Proc. Natl Acad. Sci. USA 2014; 111(37):13343-8; Gupta R et al., Appl. Microbiol. Biotechnol. 2012; 94(3):583-99; and Oakes B L et al., “Chapter Twenty-Three—Protein Engineering of Cas9 for Enhanced Function,” in Methods in Enzymology, ed. A D Jennifer, J S Erik, pp. 491-511: Academic Press (2014)) techniques can be employed to alter the primary substrate specificity of Cas9 from double-stranded DNA (dsDNA) to single-stranded RNA (ssRNA). Ideally, resulting variant(s) will retain guiding component-directed endonuclease activity but will not require addition of a PAMmer oligonucleotide to cleave EBOV RNA.

### Example 14

### Design Principles for Bacterial Targets

Traditional antibiotics (e.g., penicillin) have low molecular weights (<1500 Da) and are, therefore, able to passively diffuse into target bacteria. In contrast, polypeptide and nucleic acid-based antibiotics must be actively introduced into bacteria using one of a variety of transformation techniques, the most widely-used of which are heat shock and electroporation of competent cells. Most traditional transformation methods are not amenable to in vivo application, however, making intracellular delivery of proteins and nucleic acids to pathogenic bacteria within a mammalian host a formidable challenge that is further complicated by the fact that bacteria do not naturally endocytose macromolecules and have multiple barriers (e.g., an outer membrane, a peptidoglycan-based cell wall, and an inner membrane for Gram-negative bacteria) that hinder artificially-induced passive and active transport processes.

In an initial attempt to overcome this challenge, we modified a plasmid that encoded GFP and was labeled with Cy5 with cell-penetrating peptides (CPPs) or antimicrobial peptides (AMPs) that are known to transiently disrupt the cell wall and membranes of Gram-negative bacteria (see, e.g., 2010, Handbook of Cell-Penetrating Peptides, Second Edition: CRC Press); we then employed a high-throughput, flow cytometry-based method (see, e.g., Benincasa M et al., Antimicrob. Agents Chemother. 2009; 53(8):3501-4) to identify the CPP or AMP that maximized transport of the plasmid into Burkholderia thailandensis (Bt), a closely-related BSL-2 surrogate of Bp. Our results indicate that no currently-available CPP or AMP induces a sufficiently high degree of transformation to facilitate uptake of CRISPR expression vectors by Bp. We, therefore, propose to use bacteriophages that infect Bp with high efficiency and selectivity to introduce CRISPR constructs into the bacterium.

Bacteria have evolved CRISPR/Cas systems to provide sequence-specific protection from foreign nucleic acids, including those introduced by invading phage. In an interesting example of the evolutionary ‘arms race’ between bacteria and phage, it was recently discovered that phage use CRISPR/Cas systems to destroy phage-inhibitory chromosomal islands (PICIs) in the bacterial host in order to restore their ability to replicate (see, e.g., Seed K D et al., Nature Lett. 2013; 494:489-91). Lysogenic Bp phage can be obtained, and the phage-encoded CRISPR loci can be modified to target genes that are critical to Bp viability for destruction by a phage-encoded Cas endonuclease. Web-based programs (e.g., CRISPRFinder) can be employed to locate any and all CRISPR loci and cas genes contained within the genome of each phage, as well as any Bp strain(s) that were isolated from the same soil or water sample. We will then: (1) identify the spacer sequences in each phage-encoded CRISPR loci that have 100% identity to PICIs contained within the Bp genome; (2) use previously-reported techniques (see, e.g., Seed K D et al., PLoS Pathog. 2012; 8(9):e1002917) to introduce point mutations into each spacer sequence; and (3) perform plaque assays to identify the spacer sequences that are essential to maintain phage infectivity. Finally, we will use splicing-by-overlap-extension (SOE) PCR to replace non-essential spacer sequence(s) with guiding component(s) that target Bp genes essential for viability (e.g., purM and purN, which are critical for purine biosynthesis).

Furthermore, rational design principles include designing CRISPR components that bind to a target gene of interest found in different bacterium (e.g., both B. thailandensis and B. pseudomallei for initial in vitro screening). First, rational gene targets can be chose that either include viability genes that promote survivability of the pathogen, as well as virulence genes that promote virulence or propagation of the pathogen. Exemplary virulence genes include those that modulate transcriptional regulatory system of the pathogen (e.g., VirAG in B. pseudomallei), as well as other useful transcriptional effectors, activators, or repressors (e.g., T6SS-1, T3SS-3, TssM, BimA, BopA, and/or Bpe-AB-oprB).

Furthermore, lytic phage (e.g., that target the pathogen of interest) can be identified, and its endogenous CRISPR loci can be determined and employed. Using a synthetic approach, any CRISPR/Cas system (e.g., including an identified CRISPR loci) can be integrated into a CRISPR component. For instance, if a lytic phage is identified with endogenous CRISPR/Cas loci, then target genes can be swapped in.

Although several Bp phage have been previously isolated and characterized (see, e.g., Gatedee J et al., Virol. J. 2011; 8(1):366; Kvitko B et al., BMC Microbiol. 2012; 12(1):289; and Yordpratum U et al., FEMS Microbiol. Lett. 2011; 314(1):81-8), previous studies indicate that phage cocktails with at least one and preferably three phage for each Bp strain of concern (e.g., K96243, MSHR5855, and HPUB10134a) will be necessary to ensure efficacy of the medical countermeasure as a prophylactic or therapeutic. Therefore, we can use basic enrichment, spot test, rapid plate, and induction methods (see, e.g., Raya R R et al., “Isolation of Phage via Induction of Lysogens,” in Bacteriophages, ed. M J Clokie, A Kropinski, pp. 23-32: Humana Press (2009); and Siddiqui A et al., Appl. Microbiol. 1974; 27(1):278-80) to isolate novel Bp phage from soil and water samples; sampling location and time will be dictated by the fact that phage activity is highest when bacterial activity is beginning to wane. We will down-select phage isolates based on their ability to infect a broad range (>50%) of clinical and environmental Bp isolates without infecting other related and unrelated bacteria (e.g., B. multivorans, B. vietnamensis, B. ubonensis, and B. cepacia and Enterococcus, Escherichia, Klebsiella, Pseudomonas, Salmonella, Staphylococcus, and Streptococcus spp.) (see, e.g., Gatedee J et al. Virol. J. 2011; 8(1):366; and Yordpratum U et al., FEMS Microbiol. Lett. 2011; 314(1):81-8). Finally, we will use transmission electron microscopy (TEM), pulsed field gel electrophoresis, and Illumina's Sequencing-by-Synthesis technology to characterize the size and morphology of each phage and the size and sequence of its genome.

Since cocktails of phage (vs. individual phage) will likely be necessary to ensure efficacy against a broad-range of Bp strains, we will test the ability of each lytic and lysogenic phage to individually clear mid-log cultures of several Bp strains, including potentially K96243, MSHR5855, and HPUB10134a, at various MOIs. Models can be employed to identify five phage cocktails that each contains at least one phage for each Bp strain of concern. Resulting Bp-directed NanoCRISPRs will be tested for their ability to reduce or eliminate the growth of Bp in infected immortalized cell lines (e.g., THP-1) and primary human monocytes at various concentrations.

Briefly, host cells can be differentiated via incubation with 100 nM of phorbol myristate acetate (PMA), infected with Bp K96243 at a MOI of 10, and treated with 0.1 μg/mL of gentamicin to kill extracellular bacteria. Infected host cells will be incubated with increasing concentrations of Bp-directed NanoCRISPRs for 24 hours to construct dose-response curves and with a fixed concentration of Bp-directed NanoCRISPRs for 1-48 hours to construct time-response curves. Infected cells will be lysed by vortexing them in the presence of glass beads, and the lysate will be plated on LB agar to enumerate the number of colony-forming units (CFUs) in each sample.

### Example 15

### Design Principles for Bolstering Host Defenses By Inhibiting or Activating Gene Targets that Regulate Pathogen Recognition Pathways

Numerous studies have demonstrated that activating the innate immune system, which is fast-acting and inherently broad-spectrum, provides immediate protection against a wide variety of pathogens. EBOV, Bp, and numerous other highly virulent biothreats are adept at inhibiting innate immune responses, however, which impair the adaptive immune responses that are critical to eliminating the pathogen and protecting against re-infection (see, e.g., Wong G et al., Expert Rev. Clin. Immunol. 2014; 10(6):781-90; and Tan K S et al., J. Immunol. 2010; 184(9):5160-71). We can develop CRISPR constructs that bolster innate immunity and thereby protect host cells against EBOV, Bp, and potentially other pathogens as well. To do so, we will employ CRISPRi/a, a CRISPR/Cas9-based approach that enables specific, consistent, robust, and reversible inhibition (i) or activation (a) of target genes in mammalian cells. In this approach, catalytically inactive Cas9 is fused to a transcriptional inhibitor (Cas9i) or activator (Cas9a) protein domain, enabling inhibition or activation of gene expression upon guiding component-mediated recruitment of Cas9 to its target (see, e.g., Chavez A et al., Nat. Methods 2015; 12(4):326-8).

For preliminary analysis, we have identified four human genes (RP105, Triad3A, NLRX1, and LGP2) that, for the following reasons, are promising targets for CRISPRi-mediated protection against EBOV and Bp: (1) their gene products inhibit pathogen recognition receptor (PRR) pathways that are activated upon both viral and bacterial infection; (2) their gene products are robustly expressed in uninfected host cells; and (3) their inhibition has no measureable effect on the basal activity of the corresponding PRR pathway or other normal cellular functions. We will use bioinformatic programs to design 10 guiding components that target the promoter of each gene and clone them into expression vectors that encode Cas9i and a nuclear localization sequence to promote accumulation of Cas9i/guiding components in the nuclei of host cells. We will then use Lipofectamine® 3000 to introduce each plasmid into immortalized and primary human cells infected with a BSL-2 surrogate of EBOV (trVLPs) or Bp (Bt) and construct dose- and time-response curves to assess in vitro efficacy. The eight plasmids (per pathogen) with the lowest IC₅₀ values will be tested for efficacy and biocompatibility in human and mouse cells infected with EBOV-Zaire or Bp K9624.

To maximize the probability that we will discover efficacious host-directed CRISPR constructs, we will conduct an unbiased, genome-wide screen by: (1) designing 10 guiding component sequences targeting the promoter of each gene in the human genome (˜20,000); (2) cloning the resulting ˜200,000 guiding components into expression vectors that encode Cas9i fused to GFP and Cas9a fused to blue fluorescent protein (BFP); (3) pooling resulting vectors to produce a master library of ˜400,000 CRISPRi/a constructs; (4) introducing them into immortalized human and mouse cell lines via lentiviral transduction; (5) using FACS to isolate cells that express GFP (CRISPRi) or BFP (CRISPRa); and (6) combining GFP- and BFP-positive cells to generate a master population of CRISPRi/a-expressing host cells. We will then infect the master population with a lethal MOI of EBOV-Zaire or Bp K96243, and use PCR and next-generation sequencing (NGS) to identify and enumerate CRISPRi/a constructs in surviving host cells.

We will start by using fluorescently-labeled pathogens in combination with flow cytometry and fluorescence microscopy to determine whether each CRISPRi/a construct enables the host cell to: (1) prevent internalization of the pathogen; (2) clear internalized pathogens; or (3) harbor internalized pathogens without them causing host cell death. We will then use: (1) transcriptomic (e.g., RNA-Seq, qPCR, microarrays) and proteomic (microarrays, ELISAs, Luminex assays) techniques to quantify RNA and protein expression levels for each CRISPRi/a construct and target gene(s); and (2) a pathogen transcript enrichment technique that we recently developed (see, e.g., Bent Z W et al., PLoS ONE 2013; 8(10):e77834) to analyze pathogen expression patterns during infection, which might allow us to infer how CRISPRi/a-enabled host cells defend themselves.

### Example 16

### Combinatorial Treatments with NanoCRISPR

The NanoCRISPR delivery platform can be combined with one or more other agents to maximize efficacy. For instance, combinatorial screens can be performed to identify synergistic effects between CRISPR-based and current medical countermeasures. Efficacious pathogen and host-directed CRISPR guiding component sequences that were identified (e.g., using any methodology herein) can be screened in the presence of known antivirals or antimicrobials for synergistic effects. Identifying optimal anti-pathogen cocktails promises to not only enhance efficacy but also reduce the emergence of drug-resistant pathogens by targeting multiple orthogonal mechanisms.

For combinatorial screening involving matrices of varying concentrations of multiple guiding components and inhibitors, high-throughput screening methods can be employed, which use a robotic liquid handling system, automated microscopy, and automated image processing. Liquid handling systems allow for automated cell seeding, reagent dispensing, and gentle washing, which enable cell-based screens to be conducted in microtiter plate formats. Automated microscopy can be performed using script programs written for a microscope with an automated z-focus and stage.

Screening can include antiviral NanoCRISPR delivery platforms with other antiviral agents (e.g., ST-246 and Cidofovir). For instance, ST-246 is a small synthetic antiviral compound being developed by Siga Technologies to treat pathogenic orthopoxvirus infections in humans (see, e.g., Mucker E M et al., Antimicrob. Agents Chemother. 2013; 57:6246-53). Cidofovir (CDV) is a broad-spectrum antiviral agent that has been approved for clinical use in the treatment of cytomegalovirus retinitis but is also effective against other DNA viruses, including poxviruses (see, e.g., Smee D F et al., Antiviral Res. 2001; 52:55-62). In one instance, ST-246 and CDV in combination with CRISPR-based VacV inhibitors can be screened to find optimal concentrations of cocktails that inhibit infection and prevent resistance.

In another instance, one or more antiviral agents can be screened in combination with a multiplexed RVFV CRISPRs to identify concentrations of cocktails that inhibit infection and reduce both drug resistance and side effects. For instance, the antiviral agent can be ribavirin, a nucleoside-based, anti-metabolite prodrug that exerts a mutagenic effect on RNA viruses by facilitating G-to-A and C-to-U nucleotide transitions (see, e.g., Dietz J et al., J. Virol. 2013; 87:6172-81). It has broad-spectrum activity against RNA viruses and is a component of the FDA-approved treatment for chronic hepatitis C infection. Ribavirin has also been shown to have IC₅₀ values in the low micromolar range for RVFV (see, e.g., Peters C J et al., Antiviral Res. 1986; 6:285-97). Several side effects have been associated with ribavirin treatment, however, including hemolytic anemia, jaundice, tachycardia, and neurological perturbations.

In yet another instance, multiplexed antimicrobial CRISPRs can be screened in combination with various antibiotics and antimicrobial peptides. Individually-effective guiding component can be tested in combination with each other (i.e., thereby facilitating multiplexed gene disruption), as well as in combination with antibiotics (see, e.g., Thibault F M et al., J. Antimicrob. Chemother. 2004; 54:1134-8) and antimicrobial peptides (see, e.g., Wikraiphat C et al., FEMS Immunol. Med. Microbiol. 2009; 56:253-9) to identify concentrations of cocktails that inhibit infection and reduce resistance.

### Example 17

### Dosage and Formulation of NanoCRISPR

The NanoCRISPR delivery platform can be further studied with dosage studies that assess the concentration- and time-dependent efficacy of NanoCRISPRs in pathogen-infected cells. Such efficacy studies can guide further formulations that are efficacious in vitro and in vivo. Minimal effective doses and rising-dose toxicity can be determined using an appropriate animal model (e.g., a murine model upon lethal challenge of the target pathogen). Based on these animal studies, dosages and dosing schedules can be further optimized for primary treatment of the pathogen infection, protection against a lethal challenge, or protection against a secondary, recurrent infection based on the same pathogen.

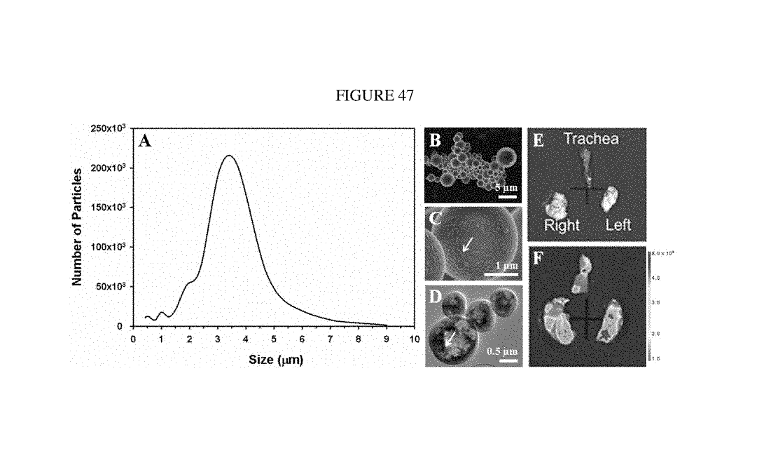

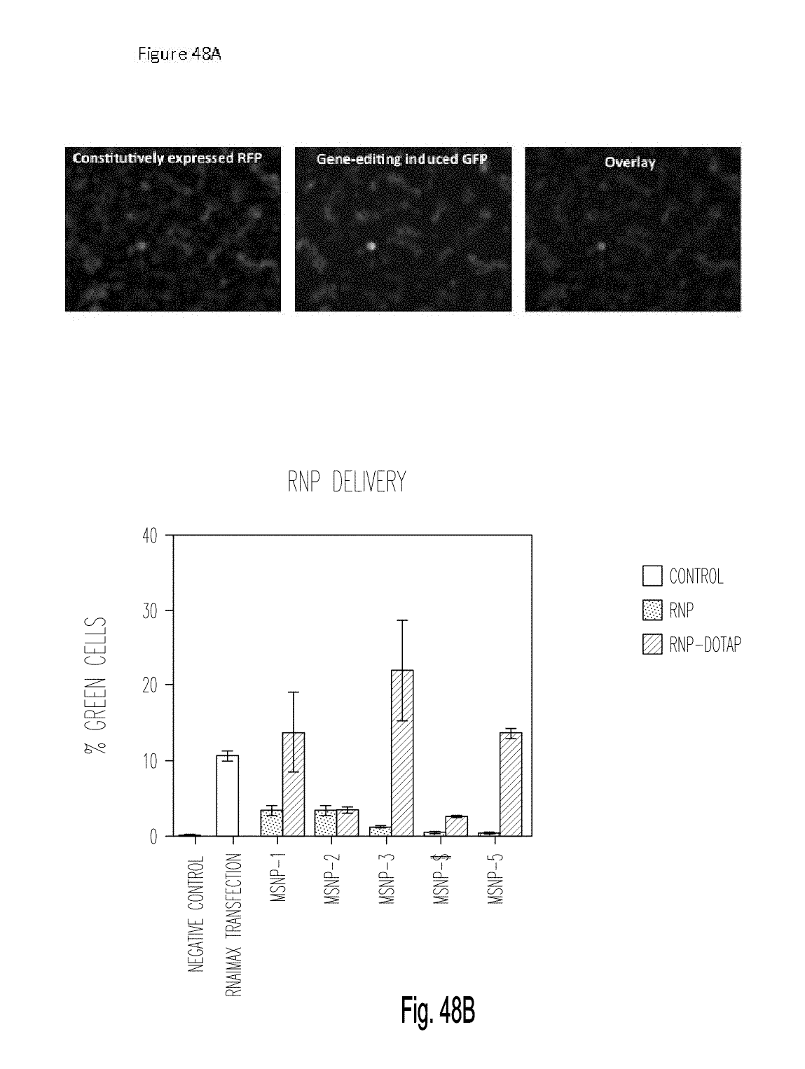

For instance, the delivery platform can be formulated in an inhalable form. Preliminary experiments indicate that we can generate dry powders that contain 45-57 wt % of SPS NPs and 5.3×10⁹ to 2.8×10¹⁰ pfu/mg of phage (FIG. 47). The inhalable dosage form can include a population of MSNPs, protocells, or silica carriers in a powder form (e.g., prepared with the spray-drying method and the like, or by using a carrier, additive, or excipient and isoniazid, urea, or mixtures thereof that can be administered via the lungs) and including an optional propellant (e.g., a liquefied gas propellant, a compressed gas, or the like). Furthermore, the inhalable dosage form can be provided as an inhalant.

### Example 18

### Protocell Delivery of Cas9 Endonuclease and Guide RNA Complexes into Mammalian Cells

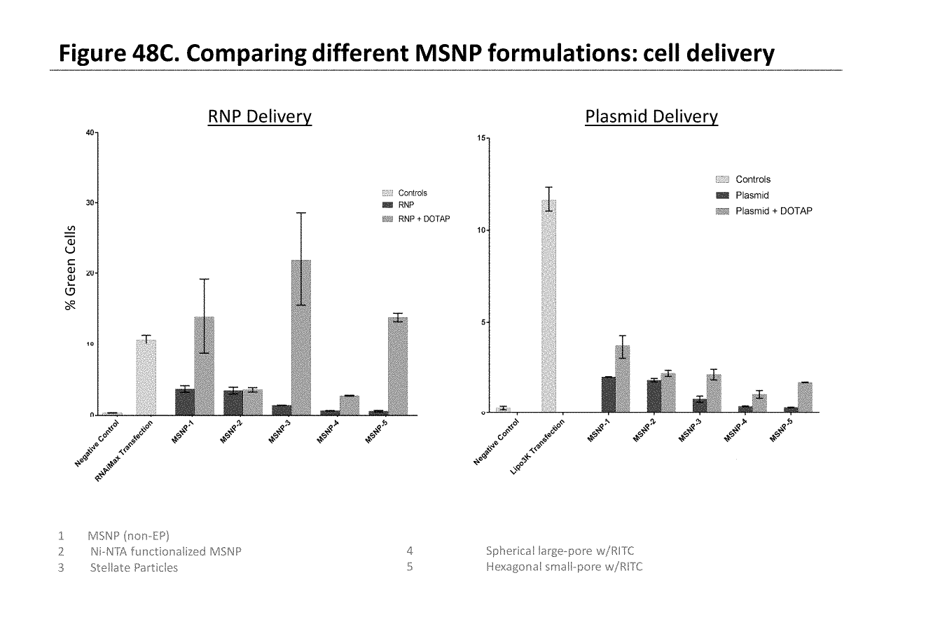

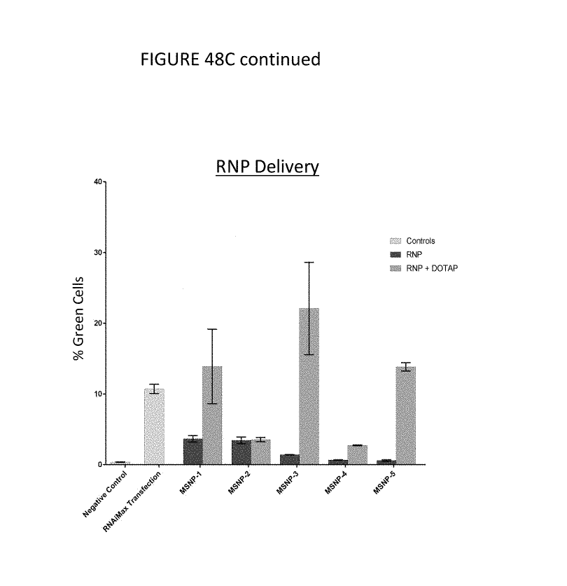

CRISPR technology uses a two-component system to induce genome-editing in target cells. One component is the Cas9 endonuclease and the standard S. pyogenes Cas9 is an about 160 kD protein. The other component is the single guide RNA (gRNA) that is a fusion of the natural dual RNA used to target the Cas9 to a protospacer sequence juxtaposed to a protospacer adjacent motif (PAM). The gRNA is approximately 100 bp in length and forms secondary structures. There are a variety of standard cell biology methods to introduce these two CRISPR components into cells, and one includes use of the Cas9/gRNA ribonucleoprotein (RNP) complex. However, the challenge in the CRISPR field is delivery of these components in vivo. In an effort to develop a CRISPR delivery system for in vivo applications, several mesoporous silica nanoparticle (MSNP)-supported lipid bilayers (SLB) or ‘protocell’ formulation were tested for in vitro editing efficiency using a cell reporter system. The cell reporter was designed to have a fluorescent read-out and express GFP upon frame-shift mutations induced through Cas9/gRNA targeting of host cell genome. The protocell formulations included a variety of MSNP cores loaded with equal amounts of CRISPR RNPs and 100% DOTAP lipid bilayers. As shown in FIG. 48, higher gene editing efficiencies using three protocell formulations as compared to the standard in vitro transfection reagent RNAimax were observed. MSNP-1 is composed of a 200 nm core with 7 nm pores, MSNP-3 is a stellate nanoparticle of 80 nm size and 12 nm pores, and the MSNP-5 is a hexagonal particle of 50 nm with 2.5 nm pores.

Thus, in one embodiment a cationic lipid formulation, e.g., one having DMPC, DSPE-PEG, DOTAP, or any combination thereof, is employed to package a negatively charged cargo, e.g., a negatively charged Cas9/gRNA complex referred to also as the ribonucleoprotein (RNP) complex, into a negatively charged mesoporous silica nanoparticle or other carrier. In one embodiment, the ratio of a combination of DMPC, DSPE-PEG, DOTAP may be about 70-80:2-6:15-25, e.g., 76:4:20

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A carrier comprising:
a biological package; and
a silica shell configured to encapsulate the biological package, wherein the silica shell comprises an outer surface and an inner surface, and wherein the inner surface is disposed to be in proximity to the biological package.

2. The carrier of claim 1, wherein the biological package comprises a dimension greater than about 20 nm.

3. The carrier of claim 1, wherein the silica shell comprises an amorphous silica.

4. The carrier of claim 3, wherein the amorphous silica is porous or non-porous.

5. The carrier of claim 1, wherein the silica shell comprises a thickness of less than about 4 nm.

6. The carrier of claim 1, further comprising:
a supported lipid layer disposed on the outer surface of the silica shell.

7. The carrier of claim 1, wherein the biological package comprises a nucleic acid and/or a polypeptide, and optionally a CRISPR component, the CRISPR component comprising:
(a) a guiding component configured to bind to a target sequence or (b) a nucleic acid that encodes a guiding component configured to bind to a target sequence, and (c) a nuclease or (d) a nucleic acid encoding a nuclease, wherein the nuclease is configured to interact with the target sequence after the guiding component binds to the target sequence.
wherein the guiding component optionally comprises:
a targeting portion comprising a nucleic acid sequence configured to bind to the target sequence; and
an interacting portion comprising a nucleic acid sequence configured to interact with the nuclease;
wherein the interacting portion comprises a structure:
A-L-B
wherein
A comprises a nucleic acid sequence having at least 80% sequence identity to any one of SEQ ID NOs:20-32 and 70 or a complement of any of these, or a fragment thereof;
L is a linker; and
B comprises a nucleic acid sequence having at least 80% sequence identity to any one of SEQ ID NOs:40-54, 60-65, and 71 or a complement of any of these, or a fragment thereof.

8. The carrier of claim 7, wherein the interacting portion comprises a nucleic acid sequence having at least 80% sequence identity to any one of SEQ ID NOs:80-93 and 100-103 or a complement of any of these, or a fragment thereof.

9. The carrier of claim 7, wherein the nuclease comprises a Cas protein comprising an amino acid sequence having at least 80% sequence identity to any one of SEQ ID NOs:110-117, or a fragment thereof or a Cas protein comprising a modification of one of more of D10A, H840A, N854A, and N863A in SEQ ID NO:110 or in an amino acid sequence sufficiently aligned with SEQ ID NO:110.

10. The carrier of claim 1, further comprising a lipid bilayer.

11. The carrier of claim 10, wherein the lipid layer comprises DOPC in combination with DOPE; DOTAP, DOPG, DOPC, or mixtures thereof; DOPG and DOPC; or cholesterol.

12. The carrier of claim 10, wherein lipid layer comprises about 5% by weight DOPE, about 5% by weight PEG, about 30% by weight cholesterol, about 60% by weight DOPC and/or DPPC.

13. The carrier of claim 12, wherein the PEG is conjugated to said DOPE.

14. The carrier of claim 1, further comprising at least one further component selected from the group consisting of a cell targeting species, a fusogenic peptide, double stranded linear DNA, plasmid nucleic acid, a drug, an imaging agent, small interfering RNA, small hairpin RNA, microRNA, or a mixture thereof, wherein one of the further components is optionally conjugated with a nuclear localization sequence.

15. The carrier of claim 14, wherein the targeting peptide comprises an amino acid sequence having at least 80% sequence identity to any one of SEQ ID NOs:126-128 or a fragment thereof or wherein the targeting peptide is a MET binding peptide comprising an amino acid sequence having at least 80% sequence identity to any one of SEQ ID NOs:121-125 or a fragment thereof.

16. The carrier of claim 14, wherein the fusogenic peptide comprises an amino acid sequence having at least 80% sequence identity to any one of SEQ ID NOs:1-6, or a fragment thereof or wherein the nuclear localization sequence comprises an amino acid sequence having at least 80% sequence identity to any one of SEQ ID NOs:9-12.

17. A pharmaceutical composition comprising a population of carriers of claim 1 in an amount effective for effecting a therapeutic effect in combination with a pharmaceutically acceptable carrier, additive or excipient.

18. The composition of claim 17, further comprising a drug which is not disposed as cargo within the carrier and optionally wherein the drug is an anticancer agent, an antiviral agent, or an antibacterial agent.

19. A method of treating cancer, a bacterial infection, or a viral infection in a patient comprising administering to said patient an effective amount of a composition of claim 18 to the patient.

20. The method of claim 19, wherein said cancer is squamous-cell carcinoma, adenocarcinoma, hepatocellular carcinoma, renal cell carcinomas, carcinoma of the bladder, bone, bowel, breast, cervix, colon (colorectal), esophagus, head, kidney, liver (hepatocellular), lung, nasopharyngeal, neck, ovary, testicles, pancreas, prostate, and stomach; a leukemia, Burkitt's lymphoma, Non-Hodgkin's lymphoma, B-cell lymphoma; malignant melanoma; myeloproliferative diseases; Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, Schwannomas, bowel cancer, breast cancer, prostate cancer, cervical cancer, uterine cancer, non-small cell lung cancer, small cell lung cancer, mixed small cell and non-small cell lung cancer, pleural mesothelioma, pleural mesothelioma, testicular cancer, thyroid cancer, and astrocytoma.


SEQUENCE LISTING (131 sequences)

SEQ ID NO: 1 - protein, 8 aa, Artificial Sequence
  A synthetic 8mer polyarginine
RRRRRRRR

SEQ ID NO: 2 - protein, 25 aa, Artificial Sequence
  A synthetic H5WYG Sequence
GLFHAIAHFIHGGWHGLIHGWYGGC

SEQ ID NO: 3 - protein, 30 aa, Artificial Sequence
  A synthetic RALA Sequence
WEARLARALARALARHLARALARALRAGEA

SEQ ID NO: 4 - protein, 30 aa, Artificial Sequence
  A synthetic KALA Sequence
WEAKLAKALAKALAKHLAKALAKALKAGEA

SEQ ID NO: 5 - protein, 30 aa, Artificial Sequence
  A synthetic GALA Sequence
WEAALAEALAEALAEHLAEALAEALEALAA

SEQ ID NO: 6 - protein, 23 aa, Artificial Sequence
  A synthetic INF7 Sequence
GLFEAIEGFIENGWEGMIDGWYG

SEQ ID NO: 7 - DNA, 9 bp, Artificial Sequence
  A synthetic target DNA Sequence
gagcatatc

SEQ ID NO: 8 - RNA, 9 nt, Artificial Sequence
  A synthetic target Sequence
gauaugcuc

SEQ ID NO: 9 - protein, 42 aa, Artificial Sequence
  A synthetic NLS sequence
GNQSSNFGPMKGGNFGGRSSGPYGGGGQYFAKPRNQGGYGGC

SEQ ID NO: 10 - protein, 7 aa, Artificial Sequence
  A synthetic NLS sequence
RRMKWKK

SEQ ID NO: 11 - protein, 7 aa, Artificial Sequence
  A synthetic NLS sequence
PKKKRKV

SEQ ID NO: 12 - protein, 16 aa, Artificial Sequence
  A synthetic NLS sequence
KRPAATKKAGQAKKKK

SEQ ID NO: 13-19 - skipped (7 entries)

SEQ ID NO: 20 - RNA, 36 nt, Artificial Sequence
  A synthetic S. pyogenes sequence
guuuuagagcuaugcuguuuugaauggucccaaaac

SEQ ID NO: 21 - RNA, 36 nt, Artificial Sequence
  A synthetic l. innocus sequence
guuuuagagcuauguuauuuugaaugcuaacaaaac

SEQ ID NO: 22 - RNA, 36 nt, Artificial Sequence
  A synthetic S. thermophilus
guuuuagagcuguguuguuucgaaugguuccaaaac

SEQ ID NO: 23 - RNA, 36 nt, Artificial Sequence
  A synthetic S. thermophilus Sequence
guuuuuguacucucaagauuuaaguaacuguacaac

SEQ ID NO: 24 - RNA, 37 nt, Artificial Sequence
  A synthetic F. novicida Sequence
cuaacaguaguuuaccaaauaauucagcaacugaaac

SEQ ID NO: 25 - RNA, 37 nt, Artificial Sequence
  A synthetic W. succinogenes Sequence
gcaacacuuuauagcaaauccgcuuagccugugaaac

SEQ ID NO: 26 - RNA, 38 nt, Artificial Sequence
  A synthetic consen 1st seq A
  misc_feature (1)...(38): n = A,T,C or G
nnnnnnnnnnununnnnnnnnnnnnnnnnnnnnnnaac

SEQ ID NO: 27 - RNA, 12 nt, Artificial Sequence
  A synthetic consen 1st seq B
  misc_feature (1)...(12): n = A,T,C or G
nnnnnnnnnnun

SEQ ID NO: 28 - RNA, 16 nt, Artificial Sequence
  A synthetic consen 1st seq C
  misc_feature (1)...(16): n = A,T,C or G
nnnnnnnnnnununnn

SEQ ID NO: 29 - RNA, 36 nt, Artificial Sequence
  A synthetic consen 1st seq D
  misc_feature (1)...(36): n = A,T,C or G
guuuungnncununnnnnuunnanunnnnnnanaac

SEQ ID NO: 30 - RNA, 12 nt, Artificial Sequence
  A synthetic consen 1st seq E
  misc_feature (1)...(12): n = A,T,C or G
guuuungnncun

SEQ ID NO: 31 - RNA, 37 nt, Artificial Sequence
  A synthetic consen 1st seq F
  misc_feature (1)...(37): n = A,T,C or G
nnaacanunnunuancaaaunnnnunancnnugaaac

SEQ ID NO: 32 - RNA, 16 nt, Artificial Sequence
  A synthetic consen 1st seq G
  misc_feature (1)...(16): n = A,T,C or G
nnaacanunnunuanc

SEQ ID NO: 33-39 - skipped (7 entries)

SEQ ID NO: 40 - RNA, 36 nt, Artificial Sequence
  A synthetic S. pyogenes Sequence
uuguuggaaccauucaaaacagcauagcaaguuaaa

SEQ ID NO: 41 - RNA, 36 nt, Artificial Sequence
  A synthetic l. innocus Sequence
auauuguuaguauucaaaauaacauagcaaguuaaa

SEQ ID NO: 42 - RNA, 36 nt, Artificial Sequence
  A synthetic S. thermophilus Sequence
gguuugaaaccauucgaaacaacacagcgaguuaaa

SEQ ID NO: 43 - RNA, 36 nt, Artificial Sequence
  A synthetic S. thermophilus Sequence
cuuacacaguuacuuaaaucuugcagaagcuacaaa

SEQ ID NO: 44 - RNA, 37 nt, Artificial Sequence
  A synthetic F. novicida Sequence
guuucaguuguuagauuauuugguauguacuuguguu

SEQ ID NO: 45 - RNA, 37 nt, Artificial Sequence
  A synthetic F. novicida Sequence
auuacagagcauuaauuauuugguacauuuauaauuu

SEQ ID NO: 46 - RNA, 37 nt, Artificial Sequence
  A synthetic W. succinogenes Sequence
uuucaaggcaucgaacggauuugcuauaaaguguugc

SEQ ID NO: 47 - RNA, 37 nt, Artificial Sequence
  A synthetic W. succinogenes Sequence
uuuguuaaagcuggaugggauuauuauagaguguugc

SEQ ID NO: 48 - RNA, 41 nt, Artificial Sequence
  A synthetic consen 2nd seq A
  misc_feature (1)...(41): n = A,T,C or G
nnnnnnnnnnnnnnnnnnnnnannnnnnannnnnnnnnnnn

SEQ ID NO: 49 - RNA, 14 nt, Artificial Sequence
  A synthetic consen 2nd seq B
  misc_feature (1)...(14): n = A,T,C or G
nannnnnnnnnnnn

SEQ ID NO: 50 - RNA, 12 nt, Artificial Sequence
  A synthetic consen 2nd seq C
  misc_feature (1)...(12): n = A,T,C or G
nnnnnnnnnnnn

SEQ ID NO: 51 - RNA, 37 nt, Artificial Sequence
  A synthetic consen 2nd seq D
  misc_feature (1)...(37): n = A,T,C or G
nnnnnnnnnnnanunnaannnnnnnagnnnnunnaaa

SEQ ID NO: 52 - RNA, 13 nt, Artificial Sequence
  A synthetic consen 2nd seq E
  misc_feature (1)...(13): n = A,T,C or G
nagnnnnunnaaa

SEQ ID NO: 53 - RNA, 38 nt, Artificial Sequence
  A synthetic consen 2nd seq F
  misc_feature (1)...(38): n = A,T,C or G
nnunnnnnnnnunnnannnnnuunnuannnnnunnnnn

SEQ ID NO: 54 - RNA, 15 nt, Artificial Sequence
  A synthetic consen 2nd seq G
  misc_feature (1)...(15): n = A,T,C or G
nnuannnnnunnnnn

SEQ ID NO: 55-59 - skipped (5 entries)

SEQ ID NO: 60 - RNA, 48 nt, Artificial Sequence
  A synthetic S. pyogenes Sequence
cuaguccguuaucaacuugaaaaaguggcaccgagucggugcuuuuuu

SEQ ID NO: 61 - RNA, 51 nt, Artificial Sequence
  A synthetic l. innocus Sequence
cuuuguccguuaucaacuuuuaauuaaguagcgcuguuucggcgcuuuuuu

SEQ ID NO: 62 - RNA, 49 nt, Artificial Sequence
  A synthetic S. thermophilus Sequence
cuuaguccguacucaacuugaaaagguggcaccgauucgguguuuuuuu

SEQ ID NO: 63 - RNA, 54 nt, Artificial Sequence
  A synthetic S. thermophilus Sequence
cuucaugccgaaaucaacacccugucauuuuauggcaggguguuuucguuauuu

SEQ ID NO: 64 - RNA, 55 nt, Artificial Sequence
  A synthetic consen tracRNA seq A
  misc_feature (1)...(55): n = A,T,C or G
cunnnunccgnnnucaacnnnnunnnannnnnungcnnngnnunnnngnuunuuu

SEQ ID NO: 65 - RNA, 10 nt, Artificial Sequence
  A synthetic consen tracRNA seq B
  misc_feature (1)...(10): n = A,T,C or G
cunnnunccg

SEQ ID NO: 66-69 - skipped (4 entries)

SEQ ID NO: 70 - RNA, 12 nt, Artificial Sequence
  A synthetic crRNA Sequence
guuuuagagcua

SEQ ID NO: 71 - RNA, 26 nt, Artificial Sequence
  A synthetic tracrRNA Sequence
uagcaaguuaaaauaaggcuaguccg

SEQ ID NO: 72-79 - skipped (8 entries)

SEQ ID NO: 80 - RNA, 38 nt, Artificial Sequence
  A synthetic Sp var. 1 Sequence
guuuuagagcuauagcaaguuaaaauaaggcuaguccg

SEQ ID NO: 81 - RNA, 79 nt, Artificial Sequence
  A synthetic cons var 1
  misc_feature (1)...(79): n = A,T,C or G
nnnnnnnnnnununnnnnnnnnnnnnnnnnnnnnnaacnnnnnnnnnnnnnnnnnnnnnannnnnnannnnnnnnnnnn

SEQ ID NO: 82 - RNA, 26 nt, Artificial Sequence
  A synthetic cons var 2A
  misc_feature (1)...(26): n = A,T,C or G
nnnnnnnnnnunnannnnnnnnnnnn

SEQ ID NO: 83 - RNA, 30 nt, Artificial Sequence
  A synthetic cons var 2B
  misc_feature (1)...(30): n = A,T,C or G
nnnnnnnnnnununnnnannnnnnnnnnnn

SEQ ID NO: 84 - RNA, 51 nt, Artificial Sequence
  A synthetic cons var 3A
  misc_feature (1)...(51): n = A,T,C or G
nnnnnnnnnnunnnnnnnnngcnnnagnuannnanauaaggcunnnunccg

SEQ ID NO: 85 - RNA, 55 nt, Artificial Sequence
  A synthetic cons var 3A
  misc_feature (1)...(55): n = A,T,C or G
nnnnnnnnnnununnnnnnnnnnngcnnnagnuannnanauaaggcunnnunccg

SEQ ID NO: 86 - RNA, 73 nt, Artificial Sequence
  A synthetic Sp var. 2 Sequence
  misc_feature (1)...(73): n = A,T,C or G
guuuungnncununnnnnuunnanunnnnnnanaacnnnnnnnnnnnanunnaannnnnnnagnnnnunnaaa

SEQ ID NO: 87 - RNA, 49 nt, Artificial Sequence
  A synthetic Sp var. 3 Sequence
  misc_feature (1)...(49): n = A,T,C or G
guuuungnncunnnnnnnnnnnnanunnaannnnnnnagnnnnunnaaa

SEQ ID NO: 88 - RNA, 25 nt, Artificial Sequence
  A synthetic Sp var. 4 Sequence
  misc_feature (1)...(25): n = A,T,C or G
guuuungnncunnagnnnnunnaaa

SEQ ID NO: 89 - RNA, 51 nt, Artificial Sequence
  A synthetic Sp var. 5 Sequence
  misc_feature (1)...(51): n = A,T,C or G
guuuungnncunnnnnnnnngcnnnagnuannnanauaaggcunnnunccg

SEQ ID NO: 90 - RNA, 74 nt, Artificial Sequence
  A synthetic Fn var. 1 Sequence
  misc_feature (1)...(74): n = A,T,C or G
nnaacanunnunuancaaaunnnnunancnnugaaacnnnnnnnnnnnanunnaannnnnnnagnnnnunnaaa

SEQ ID NO: 91 - RNA, 55 nt, Artificial Sequence
  A synthetic Fn var. 2 Sequence
  misc_feature (1)...(55): n = A,T,C or G
nnaacanunnunuancnnunnnnnnnnunnnannnnnuunnuannnnnnunnnnn

SEQ ID NO: 92 - RNA, 32 nt, Artificial Sequence
  A synthetic Fn var. 3 Sequence
  misc_feature (1)...(32): n = A,T,C or G
nnaacanunnunuancnnuannnnnnunnnnn

SEQ ID NO: 93 - RNA, 55 nt, Artificial Sequence
  A synthetic Fn var. 4 Sequence
  misc_feature (1)...(55): n = A,T,C or G
nnaacanunnunuancnnnnnnnngcnnnagnuannnanauaaggcunnnunccg

SEQ ID NO: 94-99 - skipped (6 entries)

SEQ ID NO: 100 - RNA, 218 nt, Artificial Sequence
  A synthetic Interacting portion Sequence
  misc_feature (1)...(218): n = A,T,C or G
nnnnnnnnnnnnnnnnnnnnnnnnnnnnnnnnnnnnnnnnnnnnnnnnnnnnnnnnnnnnnnnnnnnnnnnnnnnnnnnnguuuuagagcuannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnuagcaaguuaaaauaaggcuaguccg

SEQ ID NO: 101 - RNA, 219 nt, Artificial Sequence
  A synthetic Interacting portion Sequence
  misc_feature (1)...(219): n = A,T,C or G
nnnnnnnnnnnnnnnnnnnnnnnnnnnnnnnnnnnnnnnnnnnnnnnnnnnnnnnnnnnnnnnnnnnnnnnnnnnnnnnnguuuuagagcuannnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnuagcaaguuaaaauaaggcuuuguccg

SEQ ID NO: 102 - RNA, 163 nt, Artificial Sequence
  A synthetic Interacting portion Sequence
  misc_feature (1)...(163): n = A,T,C or G
nnnnnnnnnnnnnnnnnnnnnnnnnnnnnnnnnnnnnnnnnnnnnnnnnnnnnnnnnnnnnnnnnnnnnnnnnnnnnnnnguuuuagagcuagaaauagcaaguuaaaauaaggcuaguccguuaucaacuugaaaaaguggcaccgagucggugcuuuuuuu

SEQ ID NO: 103 - RNA, 163 nt, Artificial Sequence; identical to SEQ ID NO: 102
  A synthetic Interacting portion Sequence
  misc_feature (1)...(163): n = A,T,C or G

SEQ ID NO: 104-109 - skipped (6 entries)

SEQ ID NO: 110 - protein, 1369 aa, Artificial Sequence
  A synthetic SpCas9 Sequence
MDKKYSIGLDIGTNSVGWAVITDEYKVPSKKFKVLGNTDRHSIKKNLIERLLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIFHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLLYTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRGRMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMTAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 111 - protein, 1371 aa, Artificial Sequence
  A synthetic dSpCas9 Sequence
MDKKYSIGLAIGTNSVGWAVITDETKVPSKKFKVLGNTDRHSIKKNLIGALLFDSGETAEATRLKRTARRRYTRRLYNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASRVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVLYLNREDLLRKQRTFDNGSIPFQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRPHNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKEKGSQILKEHPVENTQLQNEKLYLYTLQNGRDMYVDELDINRLSDYDVDAIVPQSFLKDDSIDNKVLTRSDKNRGLYSDNVPSEEVVKKMKNYWRQLLNAKLITQRKFDNLTLYAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKEKSKKLKSVKELLGITIMERSSFEKNPIDFLEAKGYKEVKKDLIIKLPKYSLFELNGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 112 - protein, 1368 aa, Artificial Sequence
  A synthetic SpCas9 variant
MDKKYSIGLDIGTNSVGWAVITDDYKVPSKKLKGLGNTDRHGIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLADSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDATLLSDILRVNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIFYQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSDILKEYPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKVGFIKRQLVETRQITKHVAQILDSRMNTKYDENDKLIREVRVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKDPIDFLEAKGYKEVRKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 113 - protein, 1632 aa, Artificial Sequence
  A synthetic FnCas9 Sequence
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKLYLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGLYSLKDRRLYKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTARDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 114 - protein, 1410 aa, Artificial Sequence
  A synthetic StCas9 Sequence
MLFNKCIIISINLAFSNKEKCMTKPYSIGLDIGTNSVGWAVITDNYKVPSKKMKVLGNTSKKYIKKNLLGVLLFDSGITAEGLRRLKRTARRRYTRRRNRILYLQEIFSTEMATLDDAFFQRLDDSFLVPDDKRDSKYPIFGNLVEEKVYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKNLLAEFEGADYFLEKIDREDFLRKQRTFDNGSIPYQIHLEMRAILDKQAKFYPFLAKNLYERIEKILTFRIPYYVGPLARGNSDFAWSIRKRNEKITPWNFEDVIDKESSAEAFINRMTSFDLLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYKYYLQNGKDMYTGDDLDIDRLSNYDIDHIIPQAFLKDNSIDNKVLVSSASNRGKSDDFPSLEVVKKRKTFWYQLLKSKLISQRKFDNLTKAERGGLLPEDKAGFIQRQLVETRQITKHVARLLDEKFNNKKDENNRAVRTVKIITLKSTLVSQFRKDFELYKVREINDFHHAHDAYLNAVIASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFAVLVKGTIEKGAKKKITNVLEFQGISILDRINYRLYDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 115 - protein, 1393 aa, Artificial Sequence
  A synthetic StCas9 Sequence
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLTLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLKFKDLYRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGLYDMYTGDDLDIDRLSNYDIDHIIPQAFLKDNSIDNKVLVSSASNRGKSDDVPSLEVVKKRKTFWYQLLKSKLISQRLYFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSPHRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIGKGAKKKITNVLEFQGLISILDRINYRKDKLNFLLEKGYKDIELIIELPKYSLFELSDGSRRMLASILSTNNKRGEIHKGNQIFLSQLYFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 116 - protein, 1337 aa, Artificial Sequence
  A synthetic LiCas9 Sequence
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPRTIYHLREELVNSSEKADLRLVYLALAHIIKYRGNFLIEGALDTQNTSVDGIYKQFIQTYNQVFASGIEDGSLKKLEDNKDVAKILVELYVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDEVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLELYAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLFNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 117 - protein, 1061 aa, Artificial Sequence
  A synthetic WsCas9 Sequence
MIERILGVDLGISSLGWAIVEYDKDDGLAANRIIDCGVRLFTAAETPKKKESPNKARREARGIRRVLNRRRVRMNMIKKLFLRAGLIQDVDLDGEGGMFYSKANRADVWELRHDGLYRLLKGDELARVLIHIAKHRGYKFIGDDEADEESGKVKKAGVVLRQNFEAAGCRTVGEWLWRERGANGKKRNKHGDYEISIHRDLLVEEVEAIFVAQQEMRSTIATDALKAAYREIAFFVRPMQRIEKMVGHCTYFPEERRAPKSAPTAEKFIAISKFFSTVIIDNEGWEQKIIERKTLEELLDFAVSRELYVEFRHLRKFLDLSDNEIFKGLHYKGKPKTAKKREATLFDPNEPTELEFDKVEAEKKAWISLRGAAKLREALGNEFYGRFVALGKHADEATKILTYYKDEGQKRRELTKLPLEAEMVERLVKIGFSDFLKLSLKAIRDILPAMESGARYDEAVLMLGVPHKEKSAILPPLNKTDIDILNPTVIRAFAQFRKVANALVRKYGAFDRVHFELAREINTKGEIEDIKESQRKNEKERKEAADWIAETSFQVPLTRKNILKKRLYIQQDGRCAYTGDVIELERLFDEGYCEIDHILPRSRSADDSFANKVLCLARANQQKTDRTPYEWFGHDAARWNAFETRTSAPSNRVRTGKGKIDRLLKKNFDENSEMAFKDRNLNDTRYMARAIKTYCEQYWVFKNSHTKAPVQVRSGKLTSVLRYQWGLESKDRESHTHHAVDAIIIAFSTQGMVQKLSEYYRFKETHREKERPKLAVPLANFRDAVEEATRIENTETVKEGVEVKRLLISRPPRARVTGQAHEQTAKPYPRIKQVKNKKKWRLAPIDEEKFESFKADRVASANQKNFYETSTIPRVDVYHKKGKFHLVPIYLHEMVLNELPNLSLGTNPEAMDENFFKFSIFKDDLISIQTQGTPKKPAKIIMGYFKNMHGANMVLSSINNSPCEGFTCTPVSMDKKHKDKCKLCPEENRIAGRCLQGFLDYWSQEGLRPPRKEFECDQGVKFALDVKKYQIDPLGYYYGVKQEKRLGTIPQMRSAKKLVKK

SEQ ID NO: 118-120 - skipped (3 entries)

SEQ ID NO: 121 - protein, 7 aa, Artificial Sequence
  A synthetic Met Receptor Binding Peptide
ASVHFPP

SEQ ID NO: 122 - protein, 7 aa, Artificial Sequence
  A synthetic Met Receptor Binding Peptide
TATFWFQ

SEQ ID NO: 123 - protein, 7 aa, Artificial Sequence
  A synthetic Met Receptor Binding Peptide
TSPVALL

SEQ ID NO: 124 - protein, 7 aa, Artificial Sequence
  A synthetic Met Receptor Binding Peptide
IPLKVHP

SEQ ID NO: 125 - protein, 7 aa, Artificial Sequence
  A synthetic Met Receptor Binding Peptide
WPRLTNM

SEQ ID NO: 126 - protein, 12 aa, Artificial Sequence
  A synthetic SP4 Sequence
SFSIILTPILPL

SEQ ID NO: 127 - protein, 15 aa, Artificial Sequence
  A synthetic SP4 Sequence
SFSIILTPILPLGGC

SEQ ID NO: 128 - protein, 18 aa, Artificial Sequence
  A synthetic SP4 Sequence
SFSIILTPILPLEEEGGC

SEQ ID NO: 129 - protein, 38 aa, Artificial Sequence
  A synthetic polypeptide
NQSSNFGPMKGGNFGGRSSGPYGGGGQYFAKPRNQGGY

SEQ ID NO: 130 - protein, 42 aa, Artificial Sequence
  A synthetic polypeptide
DTWTGVEALIRILQQLLFIHFRIGCRHSRIGIIQQRRTRNGA

SEQ ID NO: 131 - protein, 20 aa, Artificial Sequence
  A synthetic SP4 Sequence
AKRARLSTSFNPVYPYEDES